(19) United States
(12) Patent Application Publication
(10) Pub. No.: US 2012/0009172 A1
(43) Pub. Date: Jan. 12, 2012

(54) INTRAVENOUS AND ORAL DOSING OF A DIRECT-ACTING AND REVERSIBLE P2Y12 INHIBITOR

(75) Inventors: Daniel D. Gretler, San Francisco, CA (US); Pamela B. Conley, Palo Alto, CA (US); Patrick Andre, San Mateo, CA (US); Athiwat Hutchaleelaha, Redwood City, CA (US); David R. Phillips, San Mateo, CA (US); Anjali Pandey, Fremont, CA (US); Robert M. Scarborough, Half Moon Bay, CA (US); Carroll Anna Crew Scarborough, Half Moon Bay, CA (US); Wolin Huang, Foster City, CA (US)

(73) Assignee: Portola Pharmaceuticals, Inc., So. San Francisco, CA (US)

(21) Appl. No.: 13/235,305

(22) Filed: Sep. 16, 2011

Related U.S. Application Data
(63) Continuation of application No. 12/114,630, filed on May 2, 2008, now abandoned.
(60) Provisional application No. 60/947,921, filed on Jul. 3, 2007.
(60) Provisional application No. 60/915,649, filed on May 2, 2007.

(51) Int. Cl. A61K 38/45; A61K 38/48; A61P 9/00; A61K 31/517; C07D 409/12; A61P 7/00; A61K 38/46; A61K 31/60

(52) U.S. Cl. 424/94.5; 424/94.6; 424/94.64; 514/165; 514/266.24; 544/285

(57) ABSTRACT

The invention provides methods and compositions for rapid and reversible inhibition of platelet aggregation in human subjects in need thereof by administering compounds of the formula:

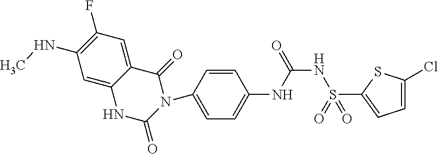

alone or in combination with a second agent which can be aspirin or a thrombolytic agent.

### CROSS-REFERENCES TO RELATED APPLICATIONS

The present application is a continuation of U.S. patent application Ser. No. 12/114,630, filed May 2, 2008, which claims the benefit of priority under 35 USC 119(e) of U.S. Provisional Application No. 60/915,649 filed on May 2, 2007 and U.S. Provisional Application No. 60/947,921 filed on Jul. 3, 2007 which are herein incorporated in their entirety by reference in their entirety for all purposes.

### BACKGROUND OF THE INVENTION

Platelet activation and aggregation play a critical role in the pathogenesis of acute coronary syndromes (ACS). The optimal antithrombotic strategy for treatment of these syndromes remains to be defined (see, Gluckman T J, Sachdev M, Schulman S P, Blumenthal R S. A simplified approach to the Management of Non-ST-segment elevation acute coronary syndromes. JAMA. 2005; 293:349-357).

ADP released from platelets propagates the thrombotic process, as it leads to platelet activation, amplification of platelet aggregation signals, and secretion of prothrombotic molecules. The ADP receptor on platelets mediating this process is the P2Y₁₂receptor, which is the target of clopidogrel (see, Dorsam R T et al., J Clin Invest. 2004 February; 113(3):340-5 for a review of the P2Y₁₂ receptor in platelet activation). Despite its widespread use, clopidogrel lacks the versatility necessary to address the different needs of coronary syndromes, due to its slow onset of action, limited inhibition of platelet aggregation, irreversibility, and large inter-individual variability in patients due to inconsistent metabolism (see, Gurbel, P. A., Bliden, K. P., Hiatt, B. L. & O'Connor, C. M. (2003). Clopidogrel for coronary stenting: response variability, drug resistance, and the effect of pretreatment platelet reactivity. Circulation 107, 2908-13; Serebruany, V. L., Steinhubl, S. R., Berger, P. B., Malinin, A. I., Bhatt, D. L. & Topol, E. J. (2005). Variability in platelet responsiveness to clopidogrel among 544 individuals. J Am Coll Cardiol 45, 246-51; and Matetzky, S., Shenkman, B., Guetta, V., Shechter, M., Bienart, R., Goldenberg, I., Novikov, I., Pres, H., Savion, N., Varon, D. & Hod, H. (2004). Clopidogrel resistance is associated with increased risk of recurrent atherothrombotic events in patients with acute myocardial infarction. Circulation 109, 3171-5).

There is an urgent need for therapeutic approaches which address the different unmet needs in ACS. The present invention meets these needs. It provides methods and compositions for rapidly and reversibly inhibiting ADP-mediated platelet aggregation in ACS.

### BRIEF SUMMARY OF THE INVENTION

The invention relates to the discovery that compounds of the Formula I and their pharmaceutically acceptable salts are reversible and rapid acting inhibitors of ADP-induced platelet aggregation in human subjects.

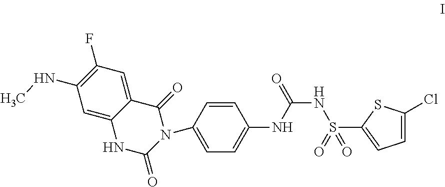

Accordingly, the invention provides compositions comprising compounds of the above formula and methods using compounds of the above formula for providing a rapid-onset and reversible inhibition of ADP-induced platelet aggregation in a human subject in need of such inhibition. The compounds for use in these methods and compositions include the crystalline solid and amorphous forms of the compounds of the above formula, including the potassium and sodium salts of [4-(6-fluoro-7-methylamino-2,4-dioxo-1,4-dihydro-2,1-quinazolin-3-yl)-phenyl]-5-chloro-thiophen-2-yl-sulfonylurea.

In some embodiments of any of the above, the subject has an acute coronary syndrome (ACS) selected from the group consisting of: acute myocardial ischemia, acute myocardial infarction, and angina. In other embodiments, the subject has a cardiovascular thrombotic disorder selected from the group consisting of a peripheral or cerebral artery occlusion. In some embodiments, the subject has a thrombotic stroke or other acute thrombotic event.

In some embodiments of the above, the subject is an ACS patient with STEMI (ST-Elevation Myocardial Infarction). In such patients, early reperfusion of the infarcted vessel is related to improved outcome. In these embodiments, the treatment resolves the ST segment elevation and/or destabilizes the thrombi or inhibits thrombosis formation or propagation.

In other aspects the invention relates to the discovery that the compounds for use according to the invention can synergize with aspirin to inhibit and to reverse platelet aggregation. Accordingly, in some embodiments the compound for use according to the invention are administered to subjects also receiving aspirin therapy. In some embodiments, compositions for use according to the invention are co-formulated with aspirin.

### BRIEF DESCRIPTION OF THE DRAWINGS

FIG. 1 provides structure of [4-(6-fluoro-7-methylamino-2,4-dioxo-1,4-dihydro-2H-quinazolin-3-yl)-phenyl]-5-chloro-thiophen-2-yl-sulfonylurea potassium and/or sodium salt.

FIG. 2a shows an X-ray powder diffraction (XRPD) of crystalline solid form A of [4-(6-fluoro-7-methylamino-2,4-dioxo-1,4-dihydro-2H-quinazolin-3-yl)-phenyl]-5-chloro-thiophen-2-yl-sulfonylurea potassium salt dihydrate. FIG. 2b shows an XRPD of crystalline solid form A of [4-(6-fluoro-7-methylamino-2,4-dioxo-1,4-dihydro-2H-quinazolin-3-yl)-phenyl]-5-chloro-thiophen-2-yl-sulfonylurea potassium salt dihydrate showing peak information.

FIG. 3a shows an XRPD of crystalline solid form B of [4-(6-fluoro-7-methylamino-2,4-dioxo-1,4-dihydro-2H-quinazolin-3-yl)-phenyl]-5-chloro-thiophen-2-yl-sulfonylurea potassium salt. FIG. 3b shows an XRPD of crystalline solid form B of [446-fluoro-7-methylamino-2,4-dioxo-1,4-dihydro-2H-quinazolin-3-yl)-phenyl]-5-chloro-thiophen-2-yl-sulfonylurea potassium salt showing peak information.

FIG. 4 shows an XRPD of the amorphous form of [4-(6-fluoro-7-methylamino-2,4-dioxo-1,4-dihydro-2H-quinazolin-3-yl)-phenyl]-5-chloro-thiophen-2-yl-sulfonylurea sodium salt.

FIG. 5 shows a Fourier-transformed infrared spectra (FT-IR) of crystalline solid form A of [4-(6-fluoro-7-methylamino-2,4-dioxo-1,4-dihydro-2H-quinazolin-3-yl)-phenyl]-5-chloro-thiophen-2-yl-sulfonylurea potassium salt dihydrate.

FIG. 6 shows a Fourier-transformed infrared spectra (FT-IR) of crystalline solid form B of [4-(6-fluoro-7-methylamino-2,4-dioxo-1,4-dihydro-2H-quinazolin-3-yl)-phenyl]-5-chloro-thiophen-2-yl-sulfonylurea potassium salt dihydrate.

FIG. 7 shows the FT-IR of an amorphous form of [4-(6-fluoro-7-methylamino-2,4-dioxo-1,4-dihydro-2H-quinazolin-3-yl)-phenyl]-5-chloro-thiophen-2-yl-sulfonylurea sodium salt.

FIG. 8 shows the ¹H-NMR of crystalline solid form A of [4-(6-fluoro-7-methylamino-2,4-dioxo-1,4-dihydro-2H-quinazolin-3-yl)-phenyl]-5-chloro-thiophen-2-yl-sulfonylurea potassium salt dihydrate.

FIG. 9 shows the ¹H-NMR of crystalline solid form B of [4-(6-fluoro-7-methylamino-2,4-dioxo-1,4-dihydro-2H-quinazolin-3-yl)-phenyl]-5-chloro-thiophen-2-yl-sulfonylurea potassium salt.

FIG. 10 shows the ¹H-NMR of amorphous form of [4-(6-fluoro-7-methylamino-2,4-dioxo-1,4-dihydro-2H-quinazolin-3-yl)-phenyl]-5-chloro-thiophen-2-yl-sulfonylurea sodium salt.

FIG. 11 provides the gravimetric vapour sorption (GVS) data of crystalline solid form A of [4-(6-fluoro-7-methylamino-2,4-dioxo-1,4-dihydro-2H-quinazolin-3-yl)-phenyl]-5-chloro-thiophen-2-yl-sulfonylurea potassium salt dihydrate.

FIG. 12a provides the gravimetric vapour sorption (GVS) data of crystalline solid form B of [4-(6-fluoro-7-methylamino-2,4-dioxo-1,4-dihydro-2H-quinazolin-3-yl)-phenyl]-5-chloro-thiophen-2-yl-sulfonylurea potassium salt dihydrate. The sample was recovered after the completion of the GVS experiment and re-examined by XRPD. The results (FIG. 12b) show that no phase change has occurred over the course of the GVS experiment. The change in intensity of the peak at ca. 5.4° 2θ, is a preferred orientation effect.

FIG. 13 provides the gravimetric vapour sorption (GVS) data of amorphous form of [4-(6-fluoro-7-methylamino-2,4-dioxo-1,4-dihydro-2H-quinazolin-3-yl)-phenyl]-5-chloro-thiophen-2-yl-sulfonylurea sodium salt.

FIG. 14 provides the differential scanning calorimetry (DSC) data of crystalline solid form A of [4-(6-fluoro-7-methylamino-2,4-dioxo-1,4-dihydro-2H-quinazolin-3-yl)-phenyl]-5-chloro-thiophen-2-yl-sulfonylurea potassium salt dihydrate.

FIG. 15 provides the TGA data of crystalline solid form A of [4-(6-fluoro-7-methylamino-2,4-dioxo-1,4-dihydro-2H-quinazolin-3-yl)-phenyl]-5-chloro-thiophen-2-yl-sulfonylurea potassium salt dihydrate.

FIG. 16 provides the DSC data of crystalline solid form B of [4-(6-fluoro-7-methylamino-2,4-dioxo-1,4-dihydro-2H-quinazolin-3-yl)-phenyl]-5-chloro-thiophen-2-yl-sulfonylurea potassium salt.

FIG. 17 provides the TGA data of crystalline solid form B of [4-(6-fluoro-7-methylamino-2,4-dioxo-1,4-dihydro-2H-quinazolin-3-yl)-phenyl]-5-chloro-thiophen-2-yl-sulfonylurea potassium salt.

FIG. 18 provides the DSC data of amorphous form of [4-(6-fluoro-7-methylamino-2,4-dioxo-1,4-dihydro-2H-quinazolin-3-yl)-phenyl]-5-chloro-thiophen-2-yl-sulfonylurea sodium salt.

FIG. 19 provides the TGA data of amorphous form of [4-(6-fluoro-7-methylamino-2,4-dioxo-1,4-dihydro-2H-quinazolin-3-yl)-phenyl]-5-chloro-thiophen-2-yl-sulfonylurea sodium salt.

FIG. 20. This figure sets forth the study objectives and design used to assess the tolerability and the pharmacokinetic (PK) and pharmacodynamic (PD) effects of single liquid oral doses of a compound of Formula I and the pharmacodynamic interaction of the compound with aspirin in healthy human subjects.

FIG. 21. This figure summarizes tolerability and safety results in the subjects.

FIG. 22. This figure presents the time course of mean plasma levels of the compound of Formula I.

FIG. 23. This figure illustrates in four panels inhibition of ADP induced platelet aggregation by a compound of Formula I. (A) Explication of aggregation of maximum amplitude and aggregation at 6 minutes. (B) Ex vivo data (mean+/−SEM) on dose dependent inhibition of ADP induced platelet aggregation measured at 6 minutes. (C) Ex vivo data (mean+/−SEM) on dose dependent inhibition of maximum amplitude ADP induced platelet aggregation. (D) Ex vivo data on the reversibility of the ADP-induced platelet aggregation inhibition at 24 hours post dose.

FIG. 24. This figure illustrates the PK-PD relationship measured ex vivo for ADP induced platelet aggregation at 6 minutes as a function of measured plasma concentration.

FIG. 25. This figure depicts the effect of aspirin and the compound of Formula I on the inhibition of collagen induced platelet aggregation.

FIG. 26. This figure illustrates (A) the Real Time Thrombosis Profiler (RTTP) Set Up; (B) the output of the assay over time; and (C) the process of thrombosis over time.

FIG. 27. This figure shows ex vivo thrombosis data using the RTTP for placebo, 10 mg, 30 mg or 100 mg of the test compound of Formula I or 30 mg of the compound with Aspirin (325 mg).

FIG. 28. This figure sets forth the study objectives and design used to assess the tolerability and the pharmacokinetic (PK) and pharmacodynamic (PD) effects of intravenous infusion of a compound of Formula I.

FIG. 29. This figure shows the plasma concentration of the studied compound of Formula I over time following i.v. infusion of 1, 3, 10, 20 and 40 mg doses in human subjects.

FIG. 30. This figure shows the inhibition of ADP-induced late platelet aggregation over time following i.v. infusion of 1, 3, 10, 20 and 40 mg doses of the compound in human subjects.

FIG. 31. This figure depicts the concentration-response for inhibition of ADP-induced platelet aggregation by the compound.

FIG. 32. This figure shows the dose-dependent inhibition of thrombosis by the compound of Formula I in human subjects i.v. infused with the compound at doses of 1, 3, 10, 20, and 40 mg.

FIG. 33. This figure shows the effects of the compound of Formula I on bleeding time are readily reversible.

FIG. 34. The effects of the compound of Formula I on thrombosis and bleeding time at 8 hours are shown for the 40 mg intravenously infused dose.

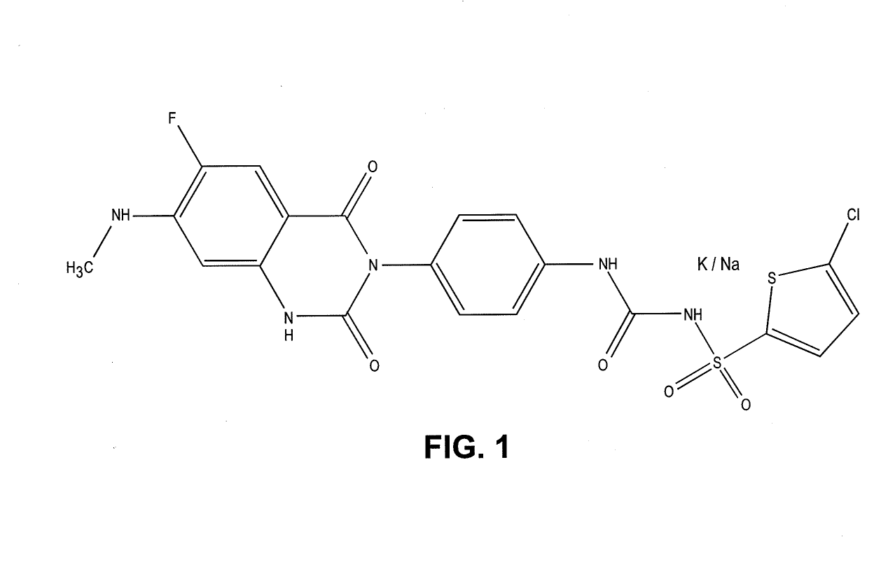

### DETAILED DESCRIPTION OF THE INVENTION

The invention relates to the Applicants discovery that the compounds for use according to the invention are rapidly acting reversible inhibitors of ADP-induced platelet aggregation in human subjects. These properties make the compounds especially useful in the treatment of acute coronary syndromes and/or in the treatment of patients needing a temporary inhibition of thrombosis formation prior to a surgical or other treatment associated with the likelihood or actual occurrence of bleeding (e.g., PCI surgery, stent insertion, joint replacement). The invention also relates to the discovery that the compounds can act synergistically with aspirin to inhibit or reverse platelet aggregation. The compounds for use according to the invention are also disclosed in PCT Patent Application No. PCT/US06/43093 which is incorporated herein by reference in its entirety.

In a first aspect, the invention provides methods of inhibiting ADP-induced platelet aggregation in a human subject in need thereof by intravenously administering to the subject a pharmaceutical composition comprising a compound of the formula:

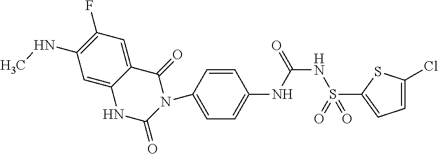

and at least one pharmaceutically acceptable excipient or carrier and in which the composition is formulated for intravenous administration. In some embodiments, the composition is formulated as a unit dose containing from 1 to 50 mg of the compound. In other embodiments, the unit dose contains from 5 to 40 mg, 10 to 30 mg, 15 to 25 mg, 25 to 45 mg, or about 20 mg, 30, 40, or 50 mg of the compound. In some embodiments, the invention provides pharmaceutical compositions which comprise the compound of Formula I or a pharmaceutically acceptable derivative of the compound of Formula I. In other embodiments, the unit dose contains from 5 to 40 mg, 10 to 30 mg, 15 to 25 mg, 25 to 45 mg, or about 20 mg, 30, 40, or 50 mg of the compound as the derivative.

In some preferred embodiments, the subject has an acute coronary syndrome. In other embodiments, the subject is individually in need of a reversible inhibition of ADP-induced platelet aggregation. For instance, the subject may need or is to be scheduled for surgery or other medical procedure associated with bleeding within one, two, three, four or five days of the administration.

In some embodiments, the composition may be administered by intravenous infusion or by an intravenous bolus. For instance, when the composition is administered as a bolus it can be administered over a period of less than 1, 2, 3, 4, or 5 minutes.

In some embodiments, the subject is treated with an i.v. dose which induces a prolonged reduction in antithrombotic effect (e.g., greater than 30, 40, 50, 60%, or 30 to 70% inhibition) at eight hours post dose and which does not have a clinically significant effect on bleeding times at eight hours post-dose. In some embodiments, the dose is from 15 to 60 mg (e.g., 15, 20, 25, 30, 35 40, 45 or 50 mg). In further embodiments, the dosage may be acute or repeated. In some embodiments, the dosage provides an antithrombotic effect without causing a clinically significant change in bleeding time at 4 to 8 hours post-dosing.

In some embodiments, the intravenous treatment inhibits ADP-induced platelet aggregation or thrombosis formation and/or propagation in the subject and/or destabilizes an existing thrombi in the subject. In some embodiments, the subject has ST-Elevation Myocardial Infarction and the treatment resolves the ST-elevation.

In some embodiments, the subject is also treated with a therapeutically effective amount of second agent to treat thrombosis or ACS. The second agent may be aspirin or a thrombolytic agent such as streptokinase, tissue plasminogen activator (TPA) or TKN. The aspirin may be administered orally. When administered in combination with a second agent, the dosage of the compound for use according to the invention optionally can be reduced. The aspirin can be given before or after the compound for use according to the invention.

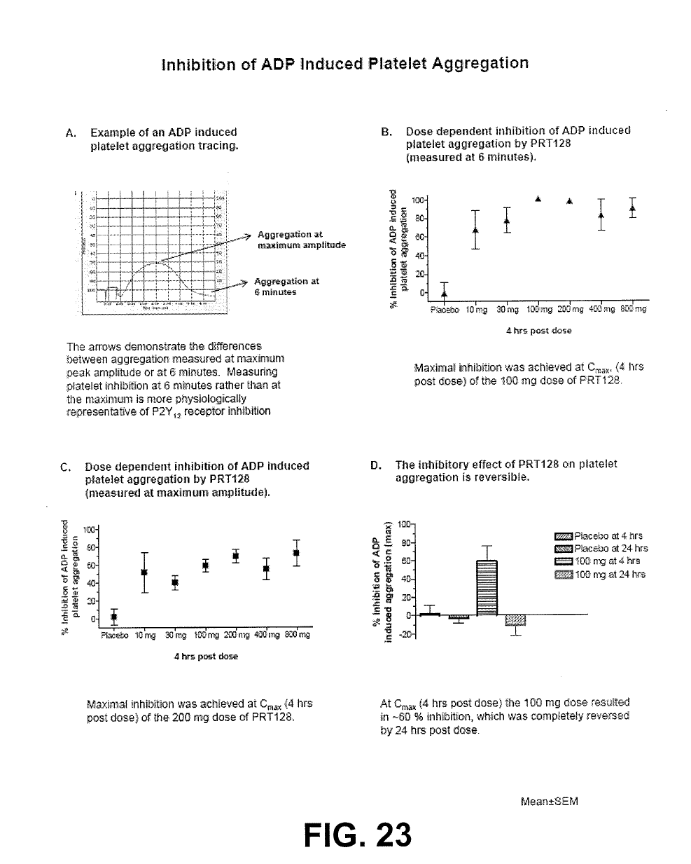

In preferred embodiments, a substantial degree of the ADP-induced platelet aggregation inhibition develops in the subject within 0.5, 1, 2, or 5 minutes after the composition is administered. The degree of inhibition which is substantial is at least 30%. In other embodiments, the degree of inhibition which is substantial is at least 50%, 70%, or 90% as determined according to the average ex vivo measurement of the ADP-induced aggregation inhibition expected for the administered dose, route and formulation in a subject of the same species, age and gender. In some embodiments, the percent inhibition is according to the extent of platelet aggregation measured at six minutes or according to the maximum aggregation as taught below and illustrated in FIG. 23A.

In another aspect the invention provides a pharmaceutical composition comprising a compound of the formula:

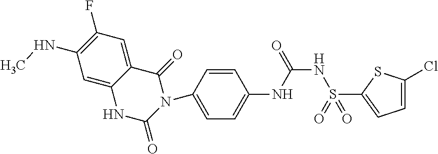

and at least one pharmaceutically acceptable excipient or carrier and in which the composition is formulated for intravenous administration. In some embodiments, the composition comprises a unit dose containing from 1 to 50 mg, 5 to 40 mg, 10 to 30 mg, or 15 to 25 mg of the compound. In some embodiments, the composition comprises a unit dose containing about 10, 20, 30, 40 or 50 mg of the compound. In some embodiments, the invention provides pharmaceutical compositions which comprise the compound of Formula I or a pharmaceutically acceptable derivative of the compound of Formula I. In other embodiments, the unit dose contains from 5 to 40 mg, 10 to 30 mg, 15 to 25 mg, 25 to 45 mg, or about 20, 30, 40, or 50 mg of the compound as the derivative.

In another aspect the invention provides a method of inhibiting ADP-induced platelet aggregation inhibition in a human subject in need thereof, said method comprising orally administering to the subject a pharmaceutical composition comprising a compound of the formula:

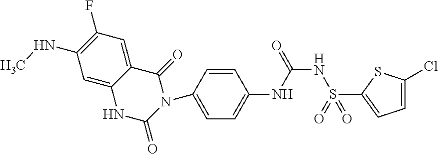

and at least one pharmaceutically acceptable excipient or carrier and in which the composition is formulated for oral administration. In some embodiments, the invention provides pharmaceutical compositions which comprise the compound of Formula I or a pharmaceutically acceptable derivative of the compound of Formula I. In some embodiments, the composition is formulated as a unit dose containing from 1 to 800 mg, 20 to 200 mg, 50 to 150 mg, 10 to 50 mg, or 20 to 40 mg of the compound or derivative. In some embodiments, the composition is in a unit dose format and contains about 30, 50, 75, 100, 125, 150, 175, or 200 mg of the compound or of the compound as derivative.

In some embodiments, the subject has an acute coronary syndrome. In some embodiments, the patient was administered an intravenous dose of the compound for use according to the invention and is being transitioned to an oral dosage regimen after having received or been on an intravenous dosage regimen. In some embodiments, the subject is in need of a reversible inhibition of ADP-induced platelet aggregation. For instance, the subject is scheduled for surgery or other medical procedure associated with bleeding within 1, 2, 3, 4, or 5 days of the administration. In some embodiments, the composition is formulated as a solid, gel, semi-liquid, or liquid. In some embodiments, the composition is formulated as a tablet, capsule, or powder. In some embodiments, the subject is also treated with a second agent used to prevent or treat thrombosis. The second agent may be aspirin or TPA, SK, or TKN. The aspirin may be administered orally. The subject was predosed with aspirin.

In some embodiments, a substantial degree of the ADP-induced platelet aggregation inhibition develops in the subject within 1 or 2 hours after the composition is orally administered. The degree of inhibition which is substantial is at least 30%. In other embodiments, the degree of inhibition which is substantial is 50%, 70%, or 90% as determined according to the average ex vivo measurement of the ADP-induced aggregation inhibition expected for the administered dose and route and formulation in a subject of the same species, age and gender. In some embodiments, the percent inhibition is according to the extent of platelet aggregation measured at six minutes or according to the maximum aggregation as taught below and illustrated in FIG. 23A.

In some embodiments, the oral administration of the compositions provides an average plasma level of the compound in the range of 400 to 4000 ng/ml, or 700 to 2000 ng/ml, or about 1000 ng/ml for at least 6 hours. In some embodiments, the oral dosage regimen is chronic and given once, twice or three times a day. In some embodiments, the oral dosage regimen provides an average 24 hour plasma concentration of the drug which is at least 200, 400, 600, 800, or 1000 ng/ml and less than 3000 ng/ml.

In some embodiments, the oral treatment inhibits ADP-induced platelet aggregation or thrombosis formation and/or propagation in the subject and/or destabilizes an existing thrombi in the subject.

In other aspect the invention provides a pharmaceutical composition comprising a compound of the formula:

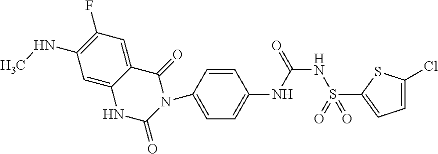

and at least one pharmaceutically acceptable excipient or carrier and in which the composition is formulated for oral administration. In some embodiments, the composition is formulated as a unit dose containing from 1 to 800 mg, 20 to 200 mg, 50 to 150 mg, 10 to 50 mg, or 20 to 40 mg of the compound. In some embodiments, the invention provides pharmaceutical compositions which comprise the compound of Formula I or a pharmaceutically acceptable derivative of the compound of Formula I. In some embodiments, the composition is formulated as a unit dose containing from 1 to 800 mg, 20 to 200 mg, 50 to 150 mg, 10 to 50 mg, or 20 to 40 mg of the compound as derivative.

### I. DEFINITIONS

In accordance with the present invention and as used herein, the following terms are defined with the following meanings, unless explicitly stated otherwise.

It is noted here that as used in this specification and the appended claims, the singular forms “a,” “an,” and “the” include plural reference unless the context clearly dictates otherwise. As such, the terms “a” (or “an”), “one or more”, and “at least one” can be used interchangeably herein.

“Anticoagulant agents” or “anticoagulants” are agents that prevent blood clot formation. Examples of anticoagulant agents include, but are not limited to, specific inhibitors of thrombin, factor IXa, factor Xa, factor XI, factor XIa, factor XIIa or factor VIIa, heparin and derivatives, vitamin K antagonists, and anti-tissue factor antibodies, as well as inhibitors of P-selectin and PSGL-1. Examples of specific inhibitors of thrombin include hirudin, bivalirudin (Angiomax®), argatroban, ximelagatran (Exanta®, see structure below), dabigatran (see structure below), AZD0837 (being studied in clinical trial A Controlled, Randomized, Parallel, Multi-Centre Feasibility Study of the Oral Direct Thrombin Inhibitor, AZD0837, Given as ER Formulation, in the Prevention of Stroke and Systolic Embolic Events in Patients With Atrial Fibrillation, Who Are Appropriate for But Unable/Unwilling to Take VKA Therapy with ClinicalTrials.gov Identifier: NCT00623779), and lepirudin (Refludan®). Examples of heparin and derivatives include unfractionated heparin (UFH), low molecular weight heparin (LMWH), such as enoxaparin (Lovenox®), dalteparin (Fragmin®), and danaparoid (Orgaran®); and synthetic pentasaccharide, such as fondaparinux (Arixtra®). Examples of vitamin K antagonists include warfarin (Coumadin®), phenocoumarol, acenocoumarol (Sintrom®), clorindione, dicumarol, diphenadione, ethyl biscoumacetate, phenprocoumon, phenindione, and tioclomarol.

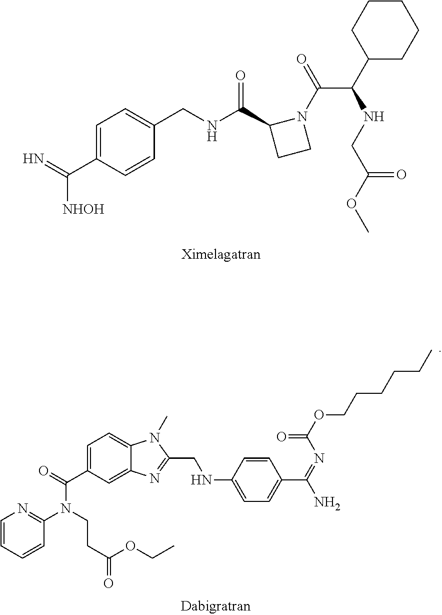

The term “factor Xa inhibitors” or “inhibitors of factor Xa” refers to compounds that can inhibit the coagulation factor Xa's activity of catalyzing conversion of prothrombin to thrombin in vitro and/or in vivo. Factor Xa is an enzyme in the coagulation pathway, and is the active component in the prothrombinase complex that catalyzes the conversion of prothrombin to thromin. Thrombin is responsible for converting fibrinogen to fibrin, and leads to formation of blood clot. Thus, inhibition of factor Xa is considered to be an effective strategy of treating and preventing thrombotic disease(s). A preferred factor Xa inhibitor inhibits thrombin formation both in vitro and in vivo. A more preferred factor Xa inhibitor shows anticoagulant efficacy in vivo. The term “specific inhibitor of factor Xa” or “specific factor Xa inhibitor” is intended to refer to factor Xa inhibitors that exhibit substantially higher inhibitory activities against factor Xa than against other enzymes or receptors of the same mammal. Preferably, a specific factor Xa inhibitor does not have significant known inhibitory activity against other enzymes or receptors in the same mammal system at its therapeutically effective concentrations.

Examples of known factor Xa inhibitors include, without limitation, fondaparinux, idraparinux, biotinylated idraparinux, enoxaparin, fragmin, NAP-5, rNAPc2, tissue factor pathway inhibitor, YM-150 (as described in e.g., Eriksson, B. I. et al, J. Thromb. Haemost. 2007, 5:1660-65, and studied in clinical trials, such as Direct Factor Xa Inhibitor YM 150 for Prevention of Venous Thromboembolism in Patients Undergoing Elective Total Hip Replacement. A Double Blind, Parallel, Dose-Finding Study in Comparison With Open Label Enoxaparin with ClinicalTrials.gov Identifier: NCT00353678), Daiichi DU-176b (as described in, e,g., E. Hylek, DU-176b, An Oral, Direct Factor Xa Antagonist, Current Opinion in Investigational Drugs 2007 8:778-783 and studied in clinical trials, such as, A Phase Ifb, Randomized, Parallel Group, Double-Blind, Double-Dummy, Multi-Center, Multi-National, Multi-Dose, Study of DU-176b Compared to Dalteparin in Patients Undergoing Elective Unilateral Total Hip Replacement with ClinicalTrials.gov Identifier: NCT00398216), betrixaban, and compounds listed in Table 1, and derivatives thereof.

TABLE 1

| Structure | Chemical Name | |
|---|---|---|
| 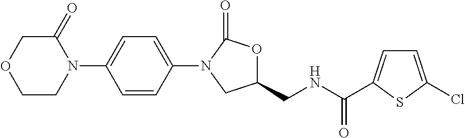 | (5S)-5-chloro-N-((2-oxo-3-(4-(3- oxomorpholino)phenyl)oxazolidin- 5-yl)methyl)thiophene-2- carboxamide | Rivaroxaban, as described in, e.g., Turpie, A.G., et al, J. Thromb. Haemost. 2005, 3(11): 2479-86 |
| 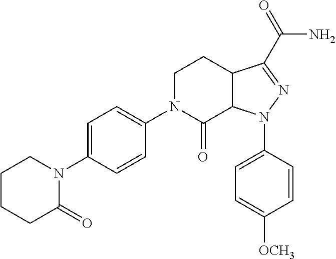 | 1-(4-methoxyphenyl)-7-oxo-6-(4- (2-oxopiperidin-1-yl)phenyl)- 3a,4,5,6,7,7a-hexahydro-1H- pyrazolo[3,4-c]pyridine-3- carboxamide | Apixaban |
| 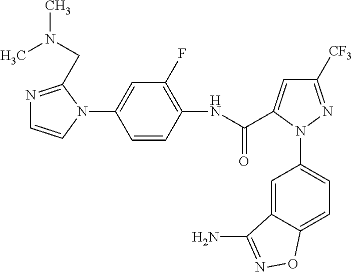 | 1-(3-aminobenzo[d]isoxazol-5-yl)- N-(4-(2-((dimethylamino)methyl)- 1H-imidazol-1-yl)-2- fluorophenyl)-3-(trifluoromethyl)- 1H-pyrazole-5-carboxamide | Razaxaban |
| 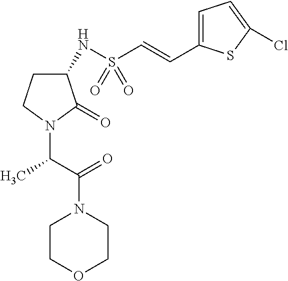 | (E)-2-(5-chlorothiophen-2-yl)-N- ((S)-1-((S)-1-morpholino-1- oxopropan-2-yl)-2-oxopyrrolidin- 3-yl)ethenesulfonamide | |
| 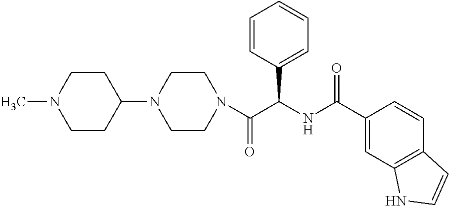 | (R)-N-(2-(4-(1-methylpiperidin-4- yl)piperazin-1-yl)-2-oxo-1- phenylethyl)-1H-indole-6- carboxamide | as described in, e.g., Agnelli, G., et al, J. Thromb. Haemost. 2007 5(4): 746-53 |
| 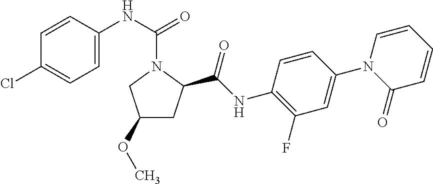 | (2R,4R)-N1-(4-chlorophenyl)-N2- (2-fluoro-4-(2-oxopyridin-1(2H)- yl)phenyl)-4-methoxypyrrolidine- 1,2-dicarboxamide | as described in, e.g., Pipeline Insight: Antithrombotics- Reaching the Untreated Prophylaxis Market, 2007 |
| 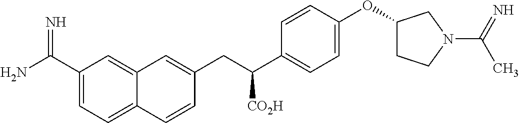 | (S)-3-(7- carbamimidoylnaphthalen-2-yl)-2- (4-((S)-1-(1-iminoethyl)pyrrolidin- 3-yloxy)phenyl)propanoic acid | as described in, e.g., Herbert, J.M., et al, J Pharmacol Exp Ther. 1996 276(3): 1030-8 |
| 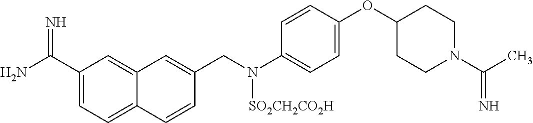 | 2-(N-((7- carbamimidoylnaphthalen-2- yl)methyl)-N-(4-(1-(1- iminoethyl)piperidin-4- yloxy)phenyl)sulfamoyl)acetic acid | as described in, e.g., Taniuchi, Y., et al, Thromb Haemost. 1998 79(3): 543-8 |
| 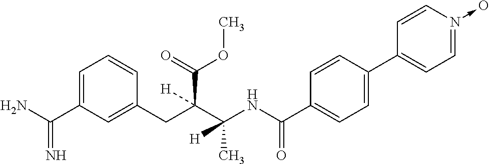 | methyl (2R, 3R)-2-(3- carbamimidoylbenzyl)-3-[[4-(1- oxidopyridin-4- yl)benzoyl]amino]butanoate | Otamixaban |

The term “factor XI inhibitors” or “inhibitors of factor XI” are compounds that can inhibit the coagulation factor XI. Upon proteolytic activation, factor XI is converted to the active enzyme factor XIa, which cleaves factor IX into factor IXa. Factor IXa then hydrolyzes factor X to factor Xa, which initiates the coagulation reactions that leads to blood clot formation as described above. An anti-factor XI antibody is a protein produced by an immune response that specifically binds factor XI, thus inhibits its activity. Some anti-factor XI antibodies are available commercially from, such as Hemetech, Inc, Ohio, USA.

“Injectable anticoagulants” are anticoagulant agents that are administrated to a mammal through injections. Examples of injectable anticoagulants are unfractionated heparin, low molecular weight heparins, and synthetic pentasaccarides.

“Antiplatelet agents” or “platelet inhibitors” are agents that block the formation of blood clots by preventing the aggregation of platelets. There are several classes of antiplatelet agents based on their activities, including, GP IIb/IIIa antagonists, such as abciximab (ReoPro®), eptifibatide (Integrilin®), and tirofiban (Aggrastat®); P2Y₁₂ receptor antagonists, such as clopidogrel (Plavix®), ticlopidine (Ticlid®), cangrelor, ticagrelor, and prasugrel; phosphodiesterase III (PDE III) inhibitors, such as cilostazol (Pletal®), dipyridamole (Persantine®) and Aggrenox® (aspirin/extended-release dipyridamole); thromboxane synthase inhibitors, such as furegrelate, ozagrel, ridogrel and isbogrel; thromboxane A2 receptor antagonists (TP antagonist), such as ifetroban, ramatroban, terbogrel, (3-{6-[(4-chlorophenylsulfonyl)amino]-2-methyl-5,6,7,8-tetrahydronaphth-1-yl}propionic acid (also known as Servier S18886, by de Recherches Internationales Servier, Courbevoie, France); thrombin receptor antagonists, such as SCH530348 (having the chemical name of ethyl (1R,3aR,4aR,6R,8aR, 9S,9aS)-9-((E)-2-(5-(3-fluorophenyl)pyridin-2-yl)vinyl)-1-methyl-3-oxododecahydronaphtho[2,3-C]furan-6-ylcarbamate, by Schering Plough Corp., New Jersey, USA, described in US20040192753A1 and US2004/0176418A1 and studied in clinical trials, such as A Multicenter, Randomized, Double-Blind, Placebo-Controlled Study to Evaluate the Safety of SCH 530348 in Subjects Undergoing Non-Emergent Percutaneous Coronary Intervention with ClinicalTrials.gov Identifier: NCT00132912); P-selectin inhibitors, such as 2-(4-chlorobenzyl)-3-hydroxy-7,8,9,10-tetrahydrobenzo[H]quinoline-4-carboxylic acid (also known as PSI-697, by Wyeth, New Jersey, USA); and non-steroidal anti-inflammatory drugs (NSAIDS), such as acetylsalicylic acid (Aspiring), resveratrol, ibuprofen (Advil®, Motrin®), naproxen (Aleve®, Naprosyn®), sulindac (Clinoril®), indomethacin (Indocin®), mefenamate, droxicam, diclofenac (Cataflam®, Voltaren®), sulfinpyrazone (Anturane®), and piroxicam (Feldene®). Among the NSAIDS, acetylsalicyclic acid (ASA), resveratrol and piroxicam are preferred. Some NSAIDS inhibit both cyclooxygenase-1 (cox-1) and cyclooxygenase-2 (cox-2), such as aspirin and ibuprofen. Some selectively inhibit cox-1, such as resveratrol, which is a reversible cox-1 inhibitor that only weakly inhibits cox-2. Beta blockers and calcium channel blockers, which are described below, also have a platelet-inhibiting effect.

The term “solvate” as used herein means a compound of the invention or a salt, thereof, that further includes a stoichiometric or non-stoichiometric amount of a solvent bound by non-covalent intermolecular forces in an amount of greater than about 0.3% when prepared according to the invention.

The term “hydrate” as used herein means a compound of the invention or a salt thereof, that further includes a stoichiometric or non-stoichiometric amount of water bound by non-covalent intermolecular forces. Hydrates are formed by the combination of one or more molecules of water with one of the substances in which the water retains its molecular state as H₂O, such combination being able to form one or more hydrate.

The term “anhydrous” as used herein means a compound of the invention or a salt thereof that contains less than about 3% by weight water or solvent when prepared according to the invention.

The term “drying” as used herein means a method of removing solvent and/or water from a compound of the invention which, unless otherwise specified, may be done at atmospheric pressure or under reduced pressure and with or without heating until the level of solvent and/or water contained reached an acceptable level.

The term “polymorphs” as used herein means crystal structures in which a compound can crystallize in different crystal packing arrangements, all of which have the same elemental composition. Different crystal forms usually have different X-ray diffraction patterns, infrared spectra, melting points/endotherm maximums, density hardness, crystal shape, optical and electrical properties, stability and solubility. Recrystallization solvent, rate of crystallization, storage temperature, and other factors may cause one crystal form to dominate.

The term “solid form” as used herein means crystal structures in which compounds can crystallize in different packing arrangements. Solid forms include polymorphs, hydrates, and solvates as those terms are used in this invention. Different solid forms, including different polymorphs, of the same compound exhibit different x-ray powder diffraction patterns and different spectra including infra-red, Raman, and solid-state NMR. Their optical, electrical, stability, and solubility properties may also differ.

The term “characterize” as used herein means to select data from an analytical measurement such as X-ray powder diffraction, infra-red spectroscopy, Raman spectroscopy, and/or solid-state NMR to distinguish one solid form of a compound from other solid forms of a compound.

As used herein, the term “preventing” refers to the prophylactic treatment of a patient in need thereof. The prophylactic treatment can be accomplished by providing an appropriate dose of a therapeutic agent to a subject at risk of suffering from an ailment, thereby substantially averting onset of the ailment.

As used herein, the term “treating” refers to providing an appropriate dose of a therapeutic agent to a subject suffering from an ailment.

The term “aspirin” or “ASA” refers to ortho-acetylsalicylic acid and the pharmaceutically acceptable formulations thereof.

As used herein, the term “therapeutically effective amount” refers to an amount of a therapeutic agent that is sufficient to affect the treatment of a subject suffering from an ailment. When a second agent is used with the compounds for use according to the invention the second compound is also used in a therapeutically effective amount. The amount(s) of one or both of agents used together may be adjusted downward when the two agents administered together act additively or syngergistically.

Acute coronary syndrome covers the spectrum of clinical conditions ranging from unstable angina to non-Q-wave myocardial infarction and Q-wave myocardial infarction. Unstable angina and non-ST-segment elevation myocardial infarction are very common manifestations of this disease. Patients having an elevated ST-segment elevation are at high risk of developing a Q-wave acute myocardial infarction or heart attack. Patients who have ischemic discomfort without an ST-segment elevation are having either unstable angina, or a non-ST-segment elevation myocardial infarction that usually leads to a non-Q-wave myocardial infarction. In some embodiments, the subject is a patient having one of the above signs of ACS. Accordingly, subjects with ACS include those whose clinical presentations cover the following range of diagnoses: unstable angina, non-ST-elevation myocardial infarction (NSTEMI), and ST-elevation myocardial infarction (STEMI).

In some embodiments, the subject is a patient having acute myocardial ischemia. Myocardial ischemia is usually due to atherosclerotic plaques, which reduce the blood supply to a portion of myocardium. Early on, the plaques may not prevent sufficient blood flow to satisfy myocardial demand. However when myocardial demand increases, the areas of narrowing may precipitate angina. For instance, this angina can be brought on by exercise, eating, and/or stress and be subsequently relieved with rest. When these symptoms remain stable in severity the condition is called chronic stable angina. However, over time, the plaques may thicken and rupture, exposing a thrombogenic surface upon which platelets can aggregate and a thrombus form to cause an unstable angina in which the symptoms of cardiac ischemia change in severity and/or duration.

The term “pharmaceutically acceptable derivatives” is meant to include salts of the active compounds which are prepared with relatively nontoxic acids or bases, depending on the particular substituents found on the compounds described herein. When compounds for use according to the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include those derived from inorganic bases such as sodium, potassium, lithium, ammonium, calcium, magnesium, iron, zinc, copper, manganese, aluminum salts and the like. Particularly preferred are the potassium and sodium salts. Salts derived from pharmaceutically acceptable organic nontoxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic nontoxic bases are isopropylamine, diethylamine, ethanolamine, trimethamine, dicyclohexylamine, choline, and caffeine. When compounds for use according to the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogen carbonic, phosphoric, monohydrogen phosphoric, dihydrogen phosphoric, sulfuric, monohydrogen sulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19; Bundgaard, H., ed., Design of Prodrugs (Elsevier Science Publishers, Amsterdam 1985)). Certain specific compounds for use according to the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

Certain preferred salt forms for the compound of Formula I are described in U.S. Patent Application Publication US 2007/0123547, titled “[4-(6-Halo-7-substituted-2,4-dioxo-1,4-dihydro-2H-quinazolin-3-yl)-phenyl]-5-chloro-thiophen-2-yl-sulfonylureas And Forms And Methods Related Thereto,” and filed Nov. 3, 2006, and claims priority from Provisional Application 60/733,650, filed on Nov. 3, 2005, both of which are hereby incorporated by reference in their entirety. Preferably, the compound forms a potassium salt (Formula I):

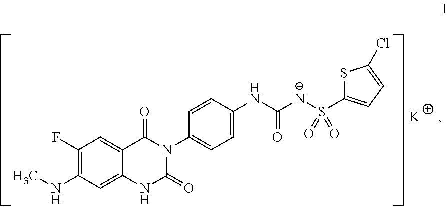

or a sodium salt (Formula II):

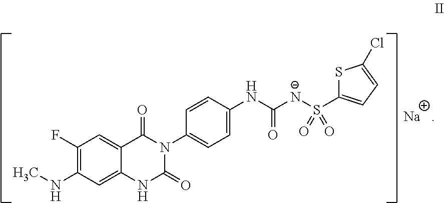

Several crystalline solid or amorphous forms of the potassium salt Formula I and sodium salt Formula II are also described in U.S. Patent Application Publication US 2007/0123547. Some preferred crystalline solid forms of the potassium salt Formula I have at least one of the following characteristics: (1) an infrared spectrum comprising peaks at about 3389 cm⁻¹ and about 1698 cm⁻¹; (2) an X-ray powder diffraction pattern comprising peaks at about 9.5 and about 25.5° 2θ; and (3) a DSC maximum endotherm at about 246° C. Among these forms, some have an infra red spectrum comprising absorption peaks at about 3559, 3389, 3324, 1698, 1623, 1563, 1510, 1448, 1431, 1403, 1383, 1308, 1269, 1206, 1174, 1123, 1091, 1072, 1030, 987, 939, 909, 871, 842, 787, 780, 769, 747, 718, 701, 690 and 667 cm⁻¹. Other preferred crystalline solid forms of the potassium salt Formula I have at least one of the following characteristics: (1) an infrared spectrum comprising peaks at about 3327 cm⁻¹ and about 1630 cm⁻¹; (2) an X-ray powder diffraction pattern comprising peaks at about 20.3 and about 25.1° 2θ; and (3) a DSC maximum endotherm at about 293° C. Among these forms, some have an infra red spectrum comprising absorption peaks at about 3584, 3327, 3189, 2935, 2257, 2067, 1979, 1903, 1703, 1654, 1630, 1590, 1557, 1512, 1444, 1429, 1406, 1375, 1317, 1346, 1317, 1288, 1276, 1243, 1217, 1182, 1133, 1182, 1133, 1093, 1072, 1033, 987, 943, 907, 883, 845, 831, 805, 776, 727, 694 and 674 cm⁻¹. Some preferred amorphous forms of the sodium salt Formula II have at least one of the following characteristics: (1) an infrared spectrum comprising peaks at about 3360, 1711, 1632, 1512, 1227, 1133 and 770 cm⁻¹; and (2) an X-ray powder diffraction pattern comprising a broad peak substantially between about 15 and about 30° 2θ. Among these forms, some have an infra red spectrum comprising absorption peaks at about 3360, 1711, 1632, 1556, 1512, 1445, 1407, 1375, 1309, 1280, 1227, 1133, 1092, 1032, 987, 905, 781, 770 and 691 cm⁻¹.

In addition to salt forms, the term “pharmaceutically acceptable derivatives” is meant to include prodrugs of the compounds for use according to the invention. “Prodrugs” of the compounds described herein are those compounds that readily undergo chemical changes under physiological conditions to provide the compounds for use according to the present invention. Additionally, prodrugs can be converted to the compounds for use according to the present invention by chemical or biochemical methods in an ex vivo environment. For example, prodrugs can be slowly converted to the compounds for use according to the present invention when placed in a transdermal patch reservoir with a suitable enzyme or chemical reagent (see Bundgaard, H., ed., Design of Prodrugs (Elsevier Science Publishers, Amsterdam 1985)).

“Pharmaceutically acceptable ester” refers to those esters which retain, upon hydrolysis of the ester bond, the biological effectiveness and properties of the carboxylic acid or alcohol and are not biologically or otherwise undesirable. For a description of pharmaceutically acceptable esters as prodrugs, see Bundgaard, H., supra. These esters are typically formed from the corresponding carboxylic acid and an alcohol. Generally, ester formation can be accomplished via conventional synthetic techniques. (See, e.g., March Advanced Organic Chemistry, 3rd Ed., p. 1157 (John Wiley & Sons, New York 1985) and references cited therein, and Mark et al., Encyclopedia of Chemical Technology, (1980) John Wiley & Sons, New York). The alcohol component of the ester will generally comprise: (i) a C₂-C₁₂ aliphatic alcohol that can or can not contain one or more double bonds and can or can not contain branched carbons; or (ii) a C₇-C₁₂ aromatic or heteroaromatic alcohols. The present invention also contemplates the use of those compositions which are both esters as described herein and at the same time are the pharmaceutically acceptable acid addition salts thereof.

“Pharmaceutically acceptable amide” refers to those amides which retain, upon hydrolysis of the amide bond, the biological effectiveness and properties of the carboxylic acid or amine and are not biologically or otherwise undesirable. For a description of pharmaceutically acceptable amides as prodrugs, see, Bundgaard, H., ed., supra. These amides are typically formed from the corresponding carboxylic acid and an amine. Generally, amide formation can be accomplished via conventional synthetic techniques. See, e.g., March et al., Advanced Organic Chemistry, 3rd Ed., p. 1152 (John Wiley & Sons, New York 1985), and Mark et al., Encyclopedia of Chemical Technology, (John Wiley & Sons, New York 1980). The present invention also contemplates the use of those compositions which are both amides as described herein and at the same time are the pharmaceutically acceptable acid addition salts thereof.

The term “pharmaceutically acceptable derivatives” is also meant to include compounds for use according to the present invention which can exist in unsolvated forms as well as solvated forms, including hydrated forms. In general, the solvated forms are equivalent to unsolvated forms and are intended to be encompassed within the scope of the present invention. Certain compounds for use according to the present invention may exist in multiple crystalline or amorphous forms. In general, all physical forms are equivalent for the uses contemplated by the present invention and are intended to be within the scope of the present invention.

Any compounds for use according to the present invention that possess asymmetric carbon atoms (optical centers) or double bonds; the racemates, diastereomers, geometric isomers and individual isomers (e.g., separate enantiomers) are all intended to be encompassed within the scope of the present invention.

The compounds for use according to the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds for use according to the present invention, whether radioactive or not, are intended to be encompassed within the scope of the present invention.

### III. PREPARATION OF COMPOUNDS FOR USE ACCORDING TO THE INVENTION

Scheme 1 illustrates a method of preparing certain compounds of Formula I wherein Ar is phenylene and R¹ is methylamino and X¹ is fluoro.

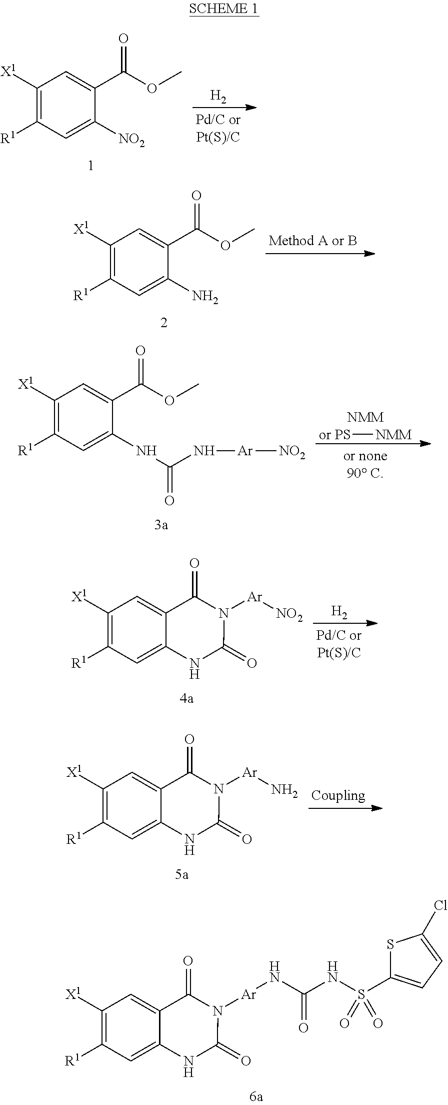

A compound of Formula I can be prepared by reducing 2-nitro-benzoic acid methyl ester compound 1 by procedures known to one skilled in the art to yield aniline 2. (See also published patent application US 2002/077486). For example, a method of nitro group reduction can be carried out by hydrogenation. The hydrogenation is carried out with a suitable catalyst (e.g., 10% Pd/C or Pt(s)/C) under hydrogen and in an appropriate solvent, typically in an alcohol, preferably ethanol at room temperature. Treating compound 2 with appropriately substituted aryl isocyanate (Method A) provides intermediate urea 3a. Alternatively, urea 3a can be formed by treating compound 2 with triphosgene in the presence of a base such as triethylamine or diisopropylethylamine in an inert solvent such as THF, dichloromethane and MeCN at appropriate temperature, preferably at 20° C., followed by substituted aniline (Method B). Urea 3a, prepared by Method A or Method B typically without further purification can be subjected to thermal or base (such as N-methyl morpholine (NMM) or polystyrene-NMM (PS-NMM) induced ring closure to provide quinazolinedione 4a. The nitro group of compound 4a can be reduced by procedures known to one skilled in the art to yield free amino group. For example, a method of reduction can be carried out by hydrogenation, with a suitable catalyst (e.g., 10% palladium on carbon) in an appropriate solvent, typically an alcohol. The formation of sulfonylurea linkage can be accomplished by treating the reduced product aniline 5a with a pre-mixed solution of substituted thiophene-2-sulfonamide, N,N′-disuccinimidyl carbonate and tetramethylguanidine in dichloromethane, followed by treatment with TFA in dichloromethane at room temperature to afford the sulfonylurea of Formula I. Alternatively, the sulfonylurea linkage can be formed by reacting the aniline 5a and 5-Chloro-thiophene-2-sulfonyl ethylcarbamate in suitable solvents, which include, but are not limited to, toluene, acetonitrile, 1,4-dioxane and DMSO.

Scheme 2 illustrates an alternative method of preparing compounds of Formula I wherein R′ is, for example, methylamino and L′ is fluoro.

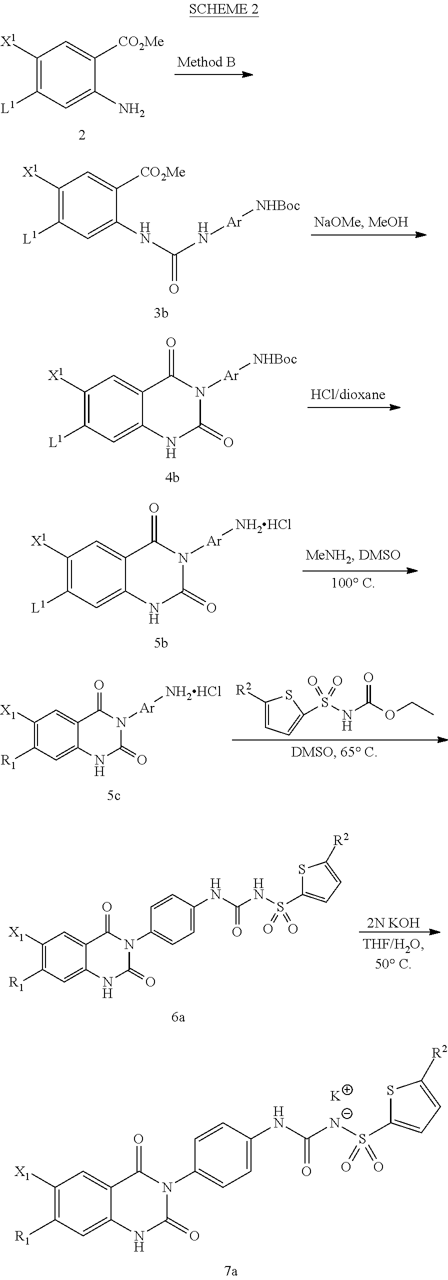

The urea 3b can be prepared by treating compound 2 with triphosgene or p-nitrophenyl chloroformate in the presence of a base, such as triethylamine and/or diisopropylethylamine, in an inert solvent, such as THF, dichloromethane and/or MeCN, at an appropriate temperature, typically at about 20° C., followed by treatment with an appropriately protected aniline (Method B). Urea 3b, typically without further purification, can be subjected to base induced ring closure to provide intermediate quinazolinedione 4b. The protecting group of compound 4b can be removed using standard techniques appropriate for the protecting group used. For example a BOC protecting group can be removed by treating compound 4b with 4N HCl in dioxane. The C-7 fluoro of compound 5b is then displaced by treatment with methylamine in DMSO at about 120° C. to afford aniline 6a. The preparation of target sulfonylurea 7a can be accomplished by treating aniline 6a with 5-chloro-thiophene-2-sulfonyl ethylcarbamate in an appropriate solvent, such as dimethyl sulfoxide, dioxane and/or acetonitrile with heating.

Scheme 3 illustrates an alternative method of preparing compounds of Formula I wherein R¹ is, for example, methylamino and L¹ is fluoro and M is K.

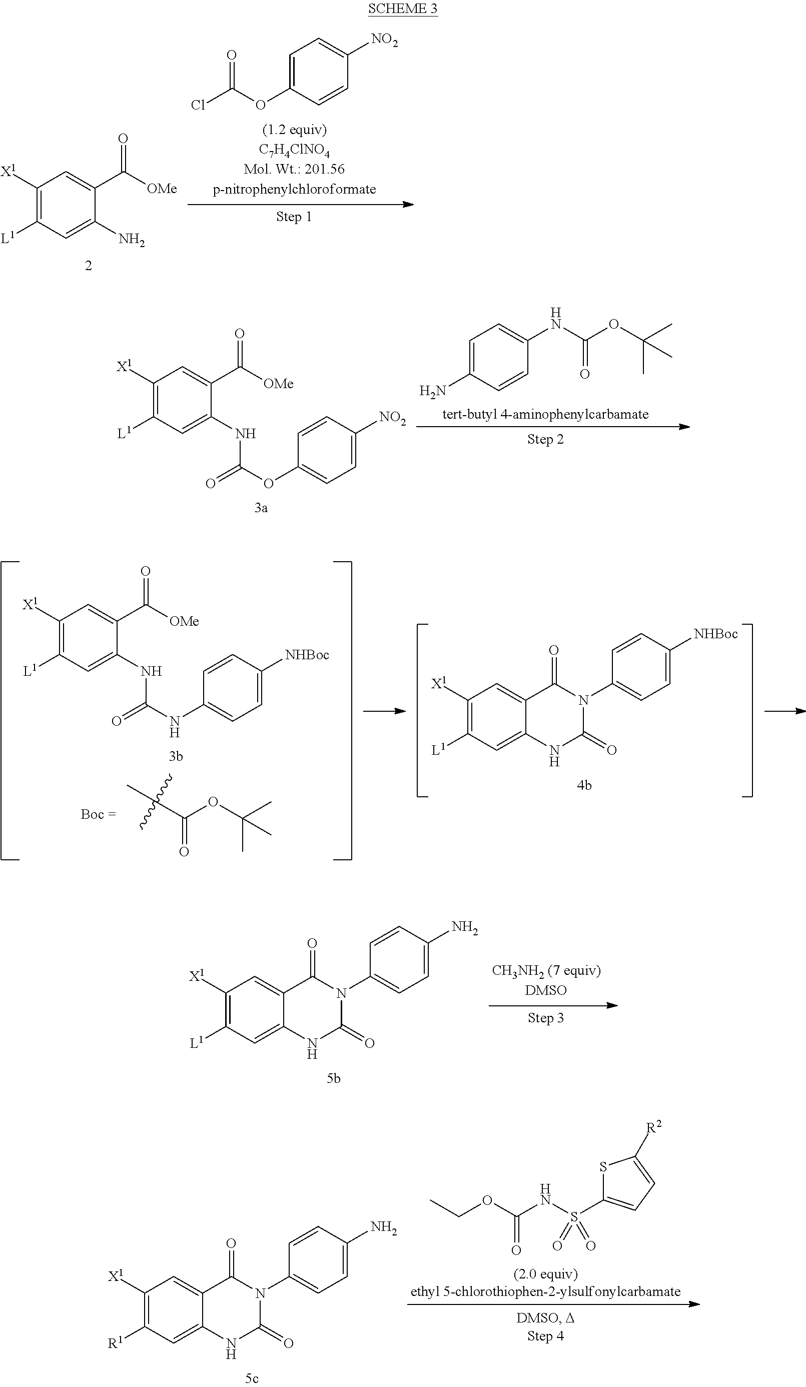

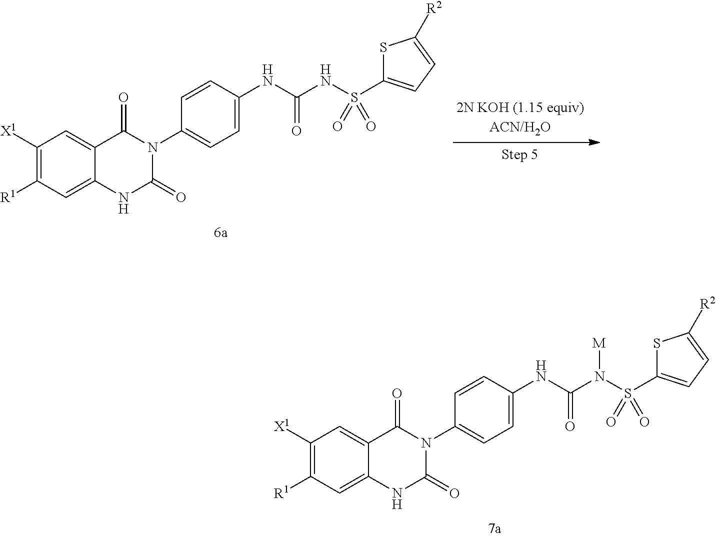

The urea 3a can be prepared by treating compound 2 with p-nitrophenylchloroformate, in an inert solvent, such as THF, dichloromethane and/or MeCN, at an appropriate temperature, typically at about 20° C., followed by treatment with an appropriately protected aniline (Method B). According to the invention, compounds of formula (I) may be further used as pharmaceutically acceptable salts e.g. 7a. Treatment of a compound for use according to the invention with an acid or base may form, respectively, a pharmaceutically acceptable acid addition salt and a pharmaceutically acceptable base addition salt, each as defined above. Various inorganic and organic acids and bases known in the art including those defined herein may be used to effect the conversion to the salt.

Compounds of formula (I) may be isolated using typical isolation and purification techniques known in the art, including, for example, chromatographic and recrystallization methods.

According to the invention, compounds of formula (I) may be further treated to form pharmaceutically acceptable salts. Treatment of a compound for use according to the invention with an acid or base may form, respectively, a pharmaceutically acceptable acid addition salt and a pharmaceutically acceptable base addition salt, each as defined above. Various inorganic and organic acids and bases known in the art including those defined herein may be used to effect the conversion to the salt.

The invention also provides for the use of pharmaceutically acceptable isomers, hydrates, and solvates of compounds of formula (I). Compounds of formula (I) may also exist in various isomeric and tautomeric forms including pharmaceutically acceptable salts, hydrates and solvates of such isomers and tautomers. For example, while some compounds are provided herein as dihydrates having two molecules of water per molecule of the compound of formula (I), the present invention also provides compounds that are anhydrous, monohydrates, trihydrates, sesquihydrates, and the like.

This invention also encompasses the use of prodrug derivatives of the compounds of formula (I). The term “prodrug” refers to a pharmacologically inactive derivative of a parent drug molecule that requires biotransformation, either spontaneous or enzymatic, within the organism to release the active drug. Prodrugs are variations or derivatives of the compounds of formula (I) for use according to this invention which have groups cleavable under metabolic conditions. Prodrugs become the compounds for use according to the invention which are pharmaceutically active in vivo when they undergo solvolysis under physiological conditions or undergo enzymatic degradation. Prodrug compounds for use according to this invention may be called single, double, triple, etc., depending on the number of biotransformation steps required to release the active drug within the organism, and indicating the number of functionalities present in a precursor-type form. Prodrug forms often offer advantages of solubility, tissue compatibility, or delayed release in the mammalian organism (Bundgard, Design of Prodrugs, pp. 7-9, 21-24, Elsevier, Amsterdam (1985); Silverman, The Organic Chemistry of Drug Design and Drug Action, pp. 352-401, Academic Press, San Diego, Calif. (1992)). Prodrugs commonly known in the art include acid derivatives well known to practitioners of the art, such as, for example, esters prepared by reaction of the parent acids with a suitable alcohol, or amides prepared by reaction of the parent acid compound with an amine, or basic groups reacted to form an acylated base derivative. Moreover, the prodrug derivatives for use according to this invention may be combined with other features herein taught to enhance bioavailability.

### IV. CRYSTALLINE SOLID AND AMORPHOUS EMBODIMENTS OF THE COMPOUNDS FOR USE ACCORDING TO THE INVENTION AND THEIR PREPARATION

The present invention also provides for the use of crystalline solid and/or amorphous forms of [4-(6-fluoro-7-methylamino-2,4-dioxo-1,4-dihydro-2H-quinazolin-3-yl)-phenyl]-5-chloro-thiophen-2-yl-sulfonylurea and processes for their preparation and pharmaceutical compositions comprising these forms. The potassium salt has the following general formula:

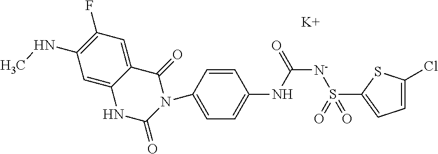

and the sodium salt has the following general formula:

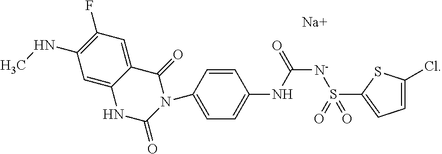

In developing a process for production of an active pharmaceutical ingredient (API), two factors are of great importance: the impurity profile and the crystal morphology of the compound. The results from the initial isolation and crystallization work showed a profile of [4-(6-fluoro-7-methylamino-2,4-dioxo-1,4-dihydro-2H-quinazolin-3-yl)-phenyl]-5-chloro-thiophen-2-yl-sulfonylurea of 99.6%. Preferably the API has levels of impurities below 0.2% and is in the most thermodynamically stable crystalline solid form. The isolation and crystallization work indicated that there was at least two crystalline solid forms of the potassium salt of [4-(6-fluoro-7-methylamino-2,4-dioxo-1,4-dihydro-2H-quinazolin-3-yl)-phenyl]-5-chloro-thiophen-2-yl-sulfonylurea (designated as Form A and B) and an amorphous form of the sodium salt of [4-(6-fluoro-7-methylamino-2,4-dioxo-1,4-dihydro-2H-quinazolin-3-yl)-phenyl]-5-chloro-thiophen-2-yl-sulfonylurea.

The solid forms for use according to the invention may be described by one or more of several techniques including X-ray powder diffraction, Raman spectroscopy, IR spectroscopy, and thermal methods. Further, combinations of such techniques may be used to describe the invention. For example, one or more X-ray powder diffraction peaks combined with one or more Raman peaks may be used to describe one or more solid forms of compounds for use according to the invention in a way that differentiates it from the other solid forms.

Although it characterizes a form, it is not necessary to rely only upon an entire diffraction pattern or spectrum to characterize a solid form. Those of ordinary skill in the pharmaceutical arts recognize that a subset of a diffraction pattern or spectrum may be used to characterize a solid form provided that subset distinguishes the solid form from the other forms being characterized. Thus, one or more X-ray powder diffraction peaks alone may be used to characterize a solid form. Likewise, one or more IR peaks alone or Raman peaks alone may be used to characterize a solid form. Such characterizations are done by comparing the X-ray, Raman, and IR data amongst the forms to determine characteristic peaks.

One may also combine data from other techniques in such a characterization. Thus, one may rely upon one or more peaks from an x-ray powder diffraction and for example, Raman or IR data, to characterize a form. For example, if one or more x-ray peaks characterize a form, one could also consider Raman or IR data to characterize the form. It is sometimes helpful to consider Raman data, for example, in pharmaceutical formulations.

The polymorphs were identified from by using two different crystallization conditions. (1) Crystalline form A was isolated after crystallization of the crude wet-cake from methanol and drying the crude wet-cake to effect solvent removal, and (2) crystalline solid form B was formed from crystallization from EtOH/H₂O or by trituration with methanol.

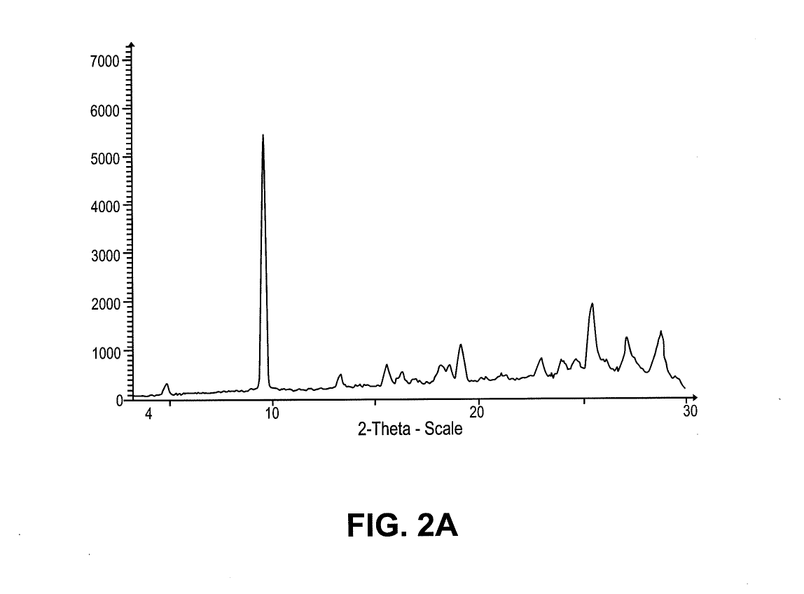

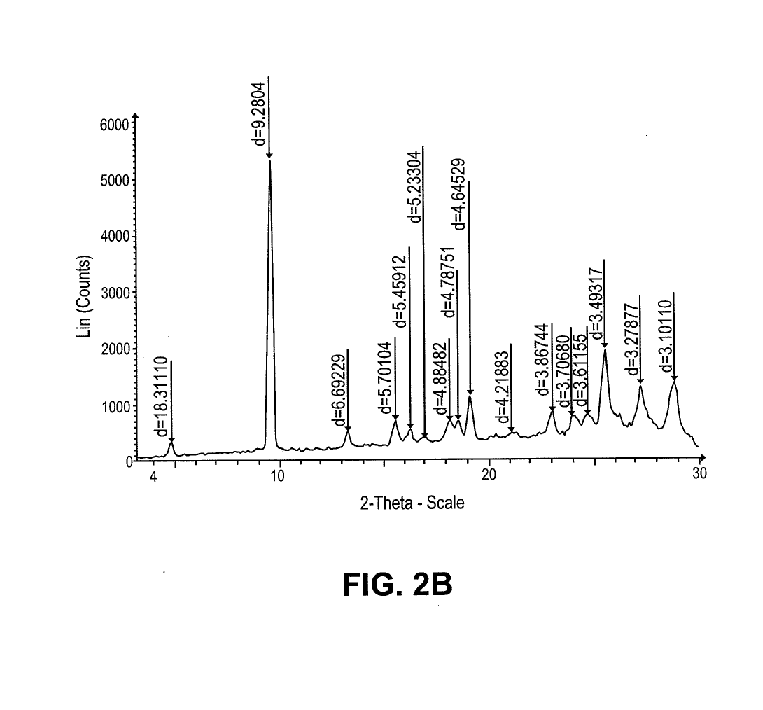

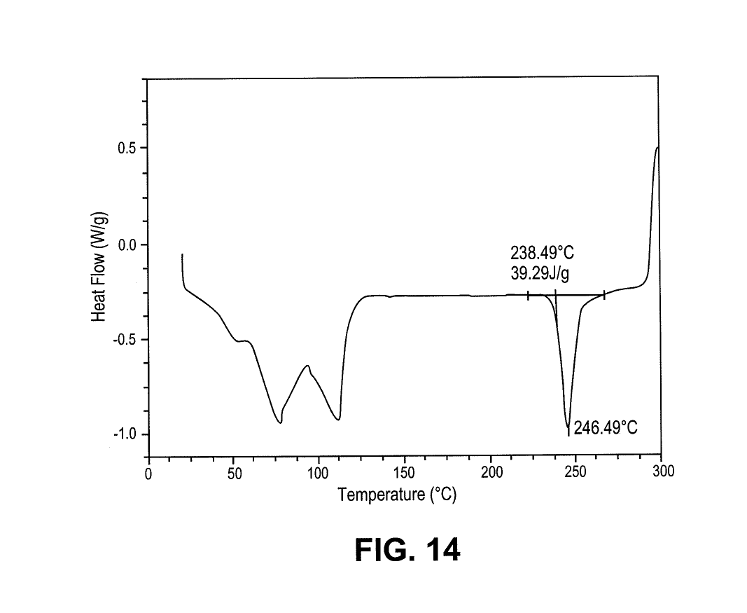

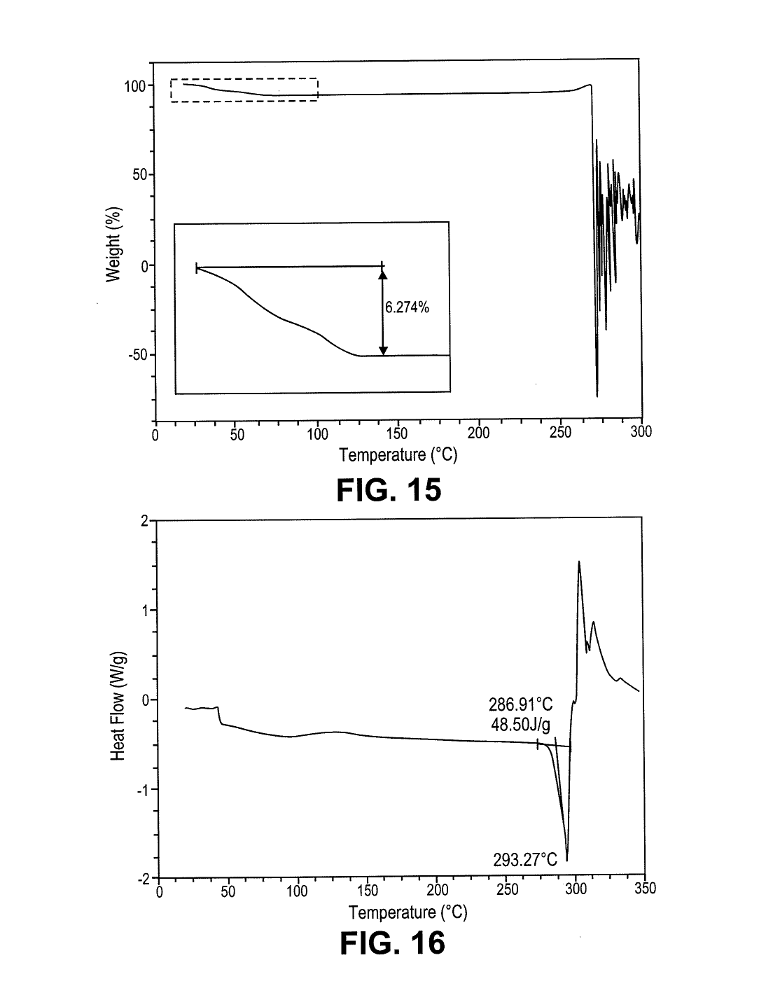

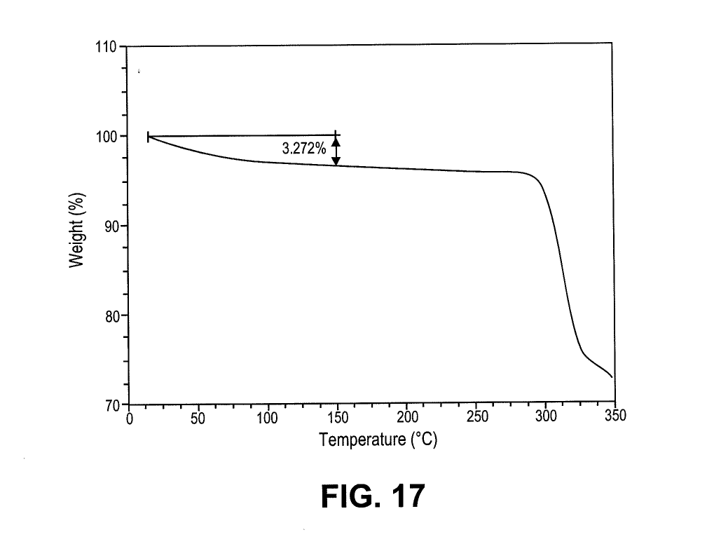

The potassium salt was suspended in methanol and then heated until a clear solution was observed. This was followed by cooling and the resulting crystalline solid was isolated and dried at room temperature under reduced pressure to give the morphologically distinct crystalline solid potassium salt/form A. FIGS. 14 and 2 respectively show the DSC trace and the X-ray powder pattern for the crystalline solid. Differential scanning calorimetry (DSC) of Form A of [4-(6-fluoro-7-methylamino-2,4-dioxo-1,4-dihydro-2H-quinazolin-3-yl)-phenyl]-5-chloro-thiophen-2-yl-sulfonylurea potassium salt defined a melt of desolvate at 238° C. A large decomposition peak was recorded, onset temperature approximately 300° C. In the DSC trace, the sharpness of the completion of melt at about 246° C. is characteristic.

In the X-ray powder diffraction pattern, the peaks at about 9.5 and 25.5 are the main features of the pattern (for a discussion of the theory of X-ray powder diffraction patterns see “X-ray diffraction procedures” by H. P. Klug and L. E. Alexander, J. Wiley, New York (1974)). The peaks at about 9.5° 2θ and 25.5° 2θ characterize Form A with respect to Form B because Form B does not have peaks to within 0.2° 2θ, twice the approximate precision of X-ray powder diffraction peaks, of the two Form A peaks. Because the typical variation in any given x-ray powder diffraction peak is on the order of 0.2° 2θ, when selecting peaks to characterize a polymorph, one selects peaks that are at least twice that value (i.e., 0.4° θ) from a peak from another polymorph. Thus, in a particular polymorph x-ray pattern, a peak that is at least 0.4° θ from a peak in another polymorph is eligible to be considered as a peak that can either alone or together with another peak be used to characterize that polymorph. Tables 1 and 2 identify the main peaks of Forms A and B. From that list, one sees that the peak at about 25.5° 2θ (on the table listed as 25.478° 2θ), when taken to one decimal point, is greater than 0.2° 2θ away from any peak in Forms B. Thus, the peak at about 25.5° 2θ can be used to distinguish Form A from Form B. The peak at about 9.5° 2θ (9.522° 2θ in Table 1) is the most intense peak in the Form A X-ray powder diffraction pattern of FIG. 2 and is more than 0.2° 2θ away from any peak in Form B. Thus, the Form A peaks at about 9.5° 2θ and 25.5° 2θ characterize Form A with respect to Form B. The solid form isolated at this stage in the process contained about 2 molecule of water to one molecule of salt.

TABLE 1
Potassium Salt Form A XRPD Peak (° 2θ) and % Intensity
Listing Data Tabulated from Figure 2b.

| Intensity | Angle | d value |
|---|---|---|
| (%) | (° 2-Theta) | (Å) |
| 100.0 | 9.522 | 9.28049 |
| 35.0 | 25.478 | 3.49317 |
| 24.2 | 28.764 | 3.10110 |
| 22.5 | 27.175 | 3.27877 |
| 20.1 | 19.090 | 4.64529 |
| 15.2 | 22.977 | 3.86744 |
| 14.4 | 24.630 | 3.61155 |
| 13.8 | 23.987 | 3.70680 |
| 12.3 | 15.530 | 5.70104 |
| 12.3 | 18.518 | 4.78751 |
| 12.1 | 18.146 | 4.88482 |
| 9.5 | 16.223 | 5.45912 |
| 8.9 | 13.219 | 6.69229 |
| 8.7 | 21.040 | 4.21883 |
| 6.8 | 16.929 | 5.23304 |
| 5.6 | 4.822 | 18.31110 |

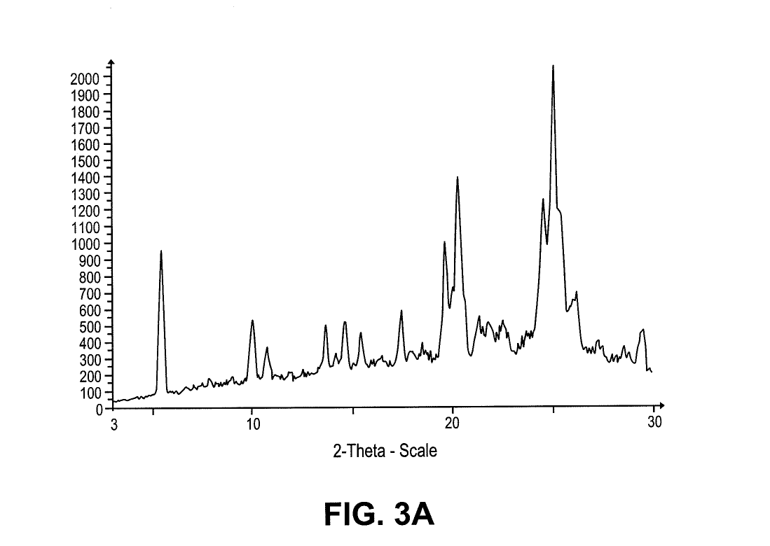

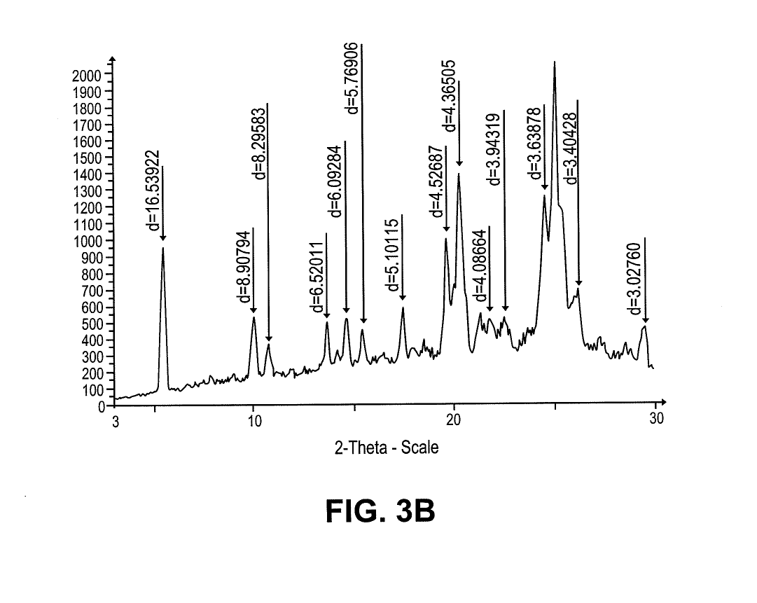

TABLE 2
Potassium Salt Form B XRPD Peak (° 2θ) and % Intensity
Listing Data Tabulated from Figure 3b.

| Intensity | Angle | d value |
|---|---|---|
| (%) | (° 2-Theta) | (Å) |
| 100.0 | 25.087 | 3.54667 |
| 70.4 | 20.328 | 4.36505 |
| 63.9 | 24.442 | 3.63878 |
| 52.9 | 5.339 | 16.53922 |
| 50.9 | 19.594 | 4.52687 |
| 34.7 | 26.155 | 3.40428 |
| 30.6 | 17.37 | 5.10115 |
| 28.6 | 21.373 | 4.15387 |
| 28.1 | 14.526 | 6.09284 |
| 27.6 | 22.53 | 3.94319 |
| 26.5 | 9.921 | 8.90794 |
| 26.5 | 21.729 | 4.08664 |
| 24.9 | 13.569 | 6.52011 |
| 23.6 | 15.346 | 5.76906 |
| 22.9 | 29.478 | 3.02760 |
| 18.9 | 10.655 | 8.29583 |

Preferred orientation can affect peak intensities, but not peak positions, in XRPD patterns. In the case of the potassium salts, preferred orientation has the most effect on the region at lower angles. Preferred orientation causes some peaks in this region to be diminished (or increased). Crystal habit does not clearly differentiate between the solid forms; a variety of habits have been observed for each form, including needles, blades, plates, and irregular-shaped particles.

Thus in one embodiment, the present invention provides for the use of [4-(6-fluoro-7-methylamino-2,4-dioxo-1,4-dihydro-2H-quinazolin-3-yl)-phenyl]-5-chloro-thiophen-2-yl-sulfonylurea potassium salt in new crystalline forms designated as Form A and Form B.

Thus in one embodiment, the invention provides for the use of [4-(6-fluoro-7-methylamino-2,4-dioxo-1,4-dihydro-2H-quinazolin-3-yl)-phenyl]-5-chloro-thiophen-2-yl-sulfonylurea potassium salt in a crystalline solid form, including a substantially pure form, which provides at least one of:

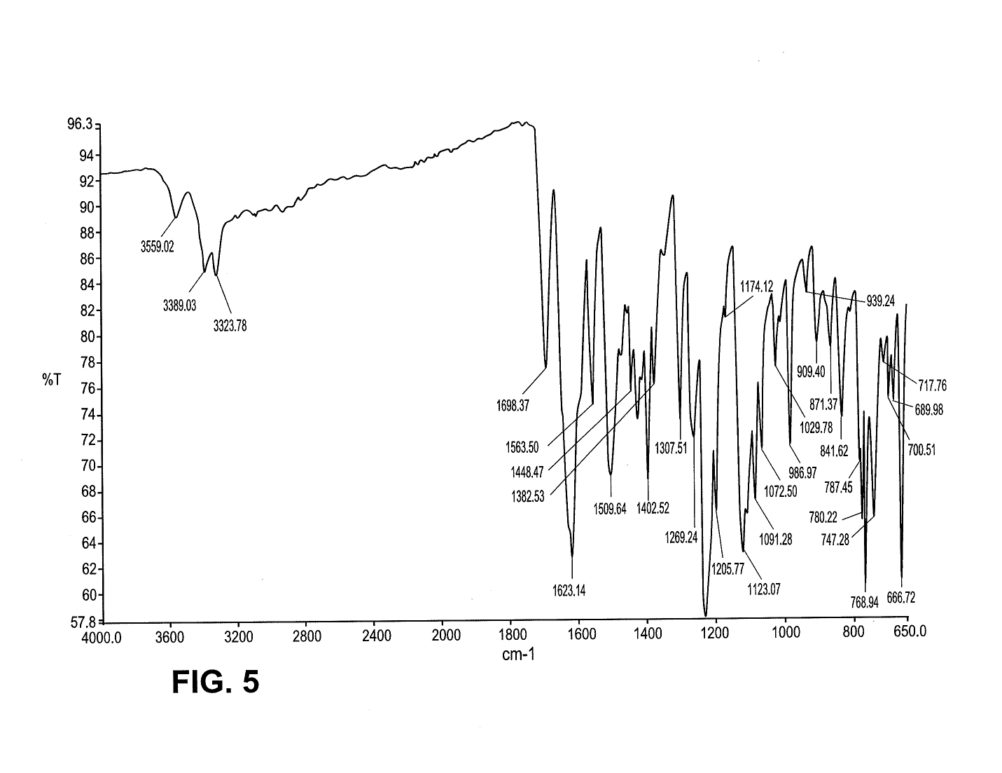

(i) an infra red spectrum substantially in accordance with FIG. 5;
(ii) an X-ray powder diffraction pattern substantially in accordance with FIG. 2; and
(iii) a DSC scan substantially in accordance with FIG. 14;
herein designated as Form A.

In another embodiment, the invention provides for the use of [4-(6-fluoro-7-methylamino-2,4-dioxo-1,4-dihydro-2H-quinazolin-3-yl)-phenyl]-5-chloro-thiophen-2-yl-sulfonylurea potassium salt in a crystalline solid form, including a substantially pure form, which provides at least one of:

(i) an infra red spectrum comprising absorption peaks at about 3559, 3389, 3324, 1698, 1623, 1563, 1510, 1448, 1431, 1403, 1383, 1308, 1269, 1206, 1174, 1123, 1091, 1072, 1030, 987, 939, 909, 871, 842, 787, 780, 769, 747, 718, 701, 690 and 667 cm⁻¹;
(ii) an X-ray powder diffraction pattern comprising peaks at about 9.5 and about 25.5° 2θ; and
(iii) a DSC maximum endotherm at about 246° C.;
herein designated as Form A.

In another embodiment, the invention provides for the use of a crystalline polymorph of [4-(6-fluoro-7-methylamino-2,4-dioxo-1,4-dihydro-2H-quinazolin-3-yl)-phenyl]-5-chloro-thiophen-2-yl-sulfonylurea potassium salt which provides an infra red spectrum containing absorption peaks at about 3559, 3389, 3324, 1698, 1623, 1563, 1510, 1448, 1431, 1403, 1383, 1308, 1269, 1206, 1174, 1123, 1091, 1072, 1030, 987, 939, 909, 871, 842, 787, 780, 769, 747, 718, 701, 690 and 667 cm⁻¹; herein designated as Form A.

In another embodiment, the invention provides for the use of [4-(6-fluoro-7-methylamino-2,4-dioxo-1,4-dihydro-2H-quinazolin-3-yl)-phenyl]-5-chloro-thiophen-2-yl-sulfonylurea potassium salt in a crystalline solid form, including a substantially pure form, which provides an X-ray powder diffraction pattern comprising peaks at about 9.5 and about 25.5° 2θ herein designated as Form A.

In another embodiment, the invention provides for the use of [4-(6-fluoro-7-methylamino-2,4-dioxo-1,4-dihydro-2H-quinazolin-3-yl)-phenyl]-5-chloro-thiophen-2-yl-sulfonylurea potassium salt in a crystalline solid form, including a substantially pure form, which provides a DSC endotherm maximum of about 246° C.; herein designated as Form A.

In another embodiment, the invention provides for the use of a crystalline polymorph of [4-(6-fluoro-7-methylamino-2,4-dioxo-1,4-dihydro-2H-quinazolin-3-yl)-phenyl]-5-chloro-thiophen-2-yl-sulfonylurea potassium salt which provides spectrum containing at least one, but fewer than the above peak listings, herein designated as Form A.

FIGS. 16 and 3 respectively show the DSC trace and the X-ray powder pattern for another crystalline solid. These results were observed when the remaining water was removed. In the DSC trace, a transition at about 293° C. is noteworthy, because Form A melts at 246° C. The peaks at about 20.3° 2θ and 25.1° 2θ in the X-ray powder diffraction pattern also characterize Form B with respect to Form A, because Form A does not have peaks to within 0.2° 2θ, the approximate precision of X-ray powder diffraction peaks, of the two characteristic Form B peaks (see Tables 1 and 2). From that list, one sees that the peaks at about 20.3° 2θ and 25.1° 2θ (in Table 2 listed as 20.328° 2θ and 25.087° 2θ, respectively), when taken to one decimal point, is greater than 0.2° 2θ away from any peak in Form A. Thus, the peaks at about 20.3° 2θ and 25.1° 2θ can be used to distinguish Form B from Form A.

Thus in one embodiment, the invention provides for the use of [4-(6-fluoro-7-methylamino-2,4-dioxo-1,4-dihydro-2H-quinazolin-3-yl)-phenyl]-5-chloro-thiophen-2-yl-sulfonylurea potassium salt in a crystalline solid form, including a substantially pure form, which provides at least one of:

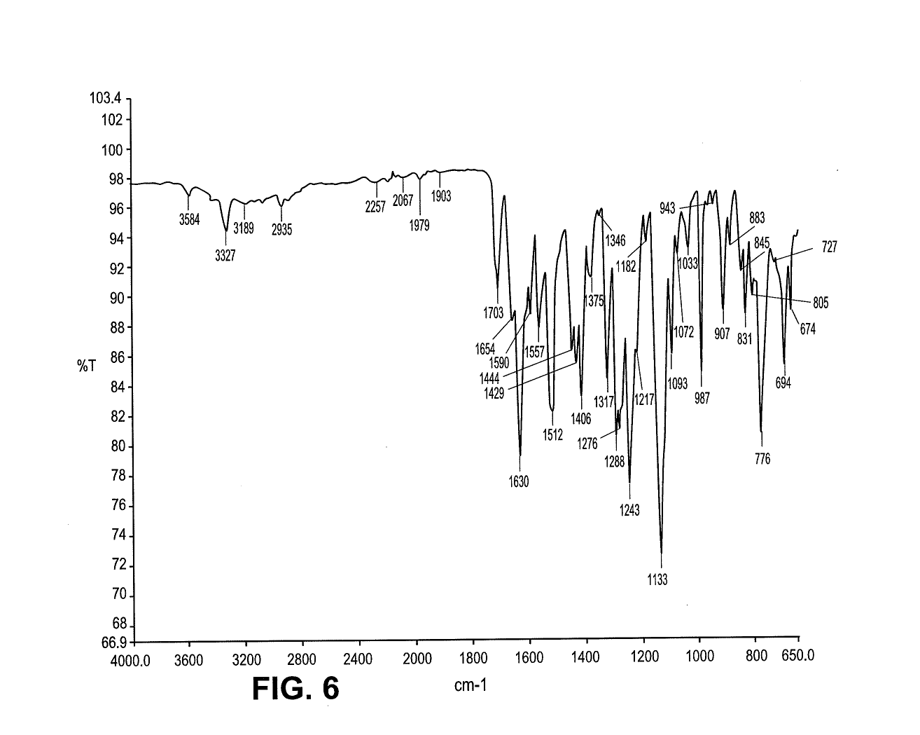

(i) an infra red spectrum substantially in accordance with FIG. 6;
(ii) an X-ray powder diffraction pattern substantially in accordance with FIG. 3; and
(iii) a DSC scan substantially in accordance with FIG. 16; herein designated as Form B.

In another embodiment, the invention provides for the use of [4-(6-fluoro-7-methylamino-2,4-dioxo-1,4-dihydro-2H-quinazolin-3-yl)-phenyl]-5-chloro-thiophen-2-yl-sulfonylurea potassium salt in a crystalline solid form, including a substantially pure form, which (i) an infra red spectrum comprising absorption peaks at about 3584, 3327, 3189, 2935, 2257, 2067, 1979, 1903, 1703, 1654, 1630, 1590, 1557, 1512, 1444, 1429, 1406, 1375, 1317, 1346, 1317, 1288, 1276, 1243, 1217, 1182, 1133, 1182, 1133, 1093, 1072, 1033, 987, 943, 907, 883, 845, 831, 805, 776, 727, 694 and 674 cm⁻¹; (ii) an X-ray powder diffraction pattern comprising peaks at about 20.3° 2θ and about 25.1° 2θ; and

(iii) a DSC maximum endotherm at about 293° C.; herein designated as Form B.

In another embodiment, the invention provides for the use of [4-(6-fluoro-7-methylamino-2,4-dioxo-1,4-dihydro-2H-quinazolin-3-yl)-phenyl]-5-chloro-thiophen-2-yl-sulfonylurea potassium salt in a crystalline solid form, including a substantially pure form, wherein the compound provides an X-ray powder diffraction pattern comprising peaks at about 20.3° 2θ and 25.1° 2θ; herein designated as Form B.

In another embodiment the present invention provides for the use of [4-(6-fluoro-7-methylamino-2,4-dioxo-1,4-dihydro-2H-quinazolin-3-yl)-phenyl]-5-chloro-thiophen-2-yl-sulfonylurea sodium salt in an amorphous form.

In one embodiment, the invention provides for the use of a form of [4-(6-fluoro-7-methylamino-2,4-dioxo-1,4-dihydro-2H-quinazolin-3-yl)-phenyl]-5-chloro-thiophen-2-yl-sulfonylurea sodium salt which provides at least one of:

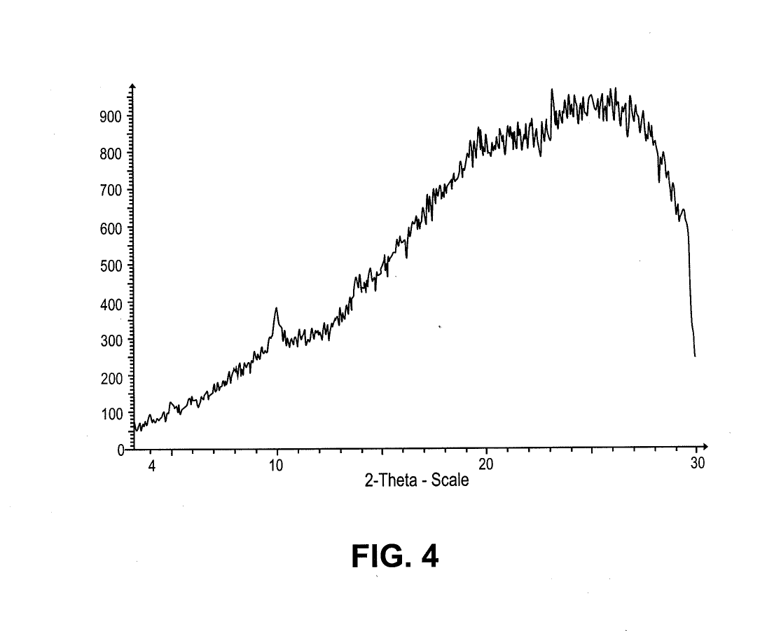

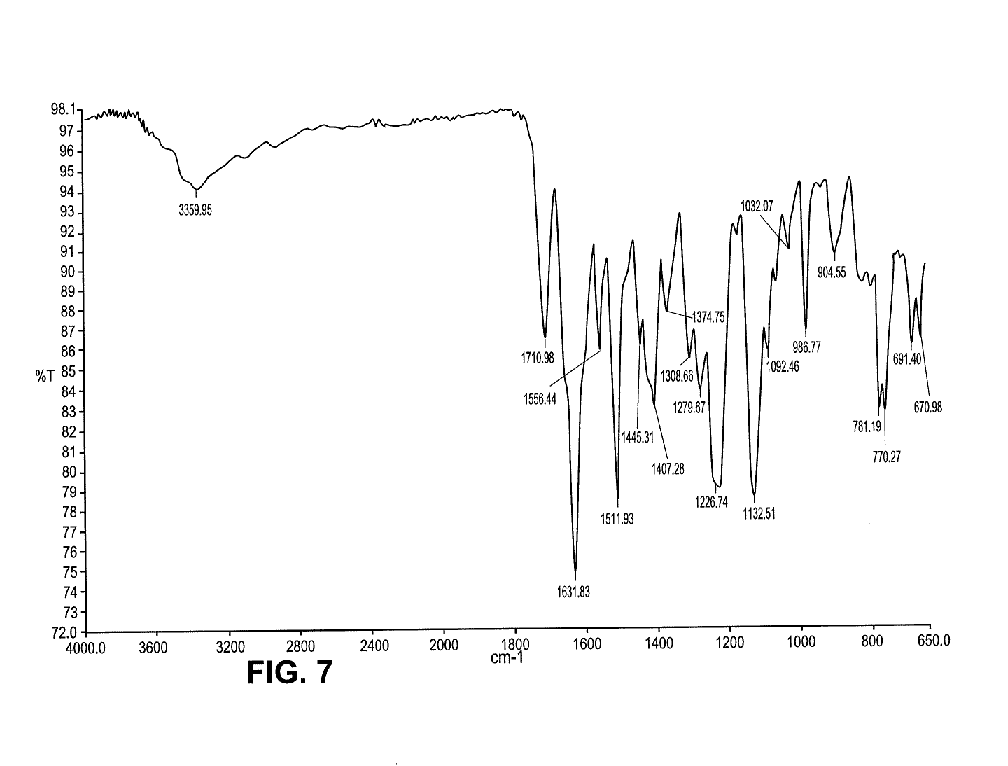

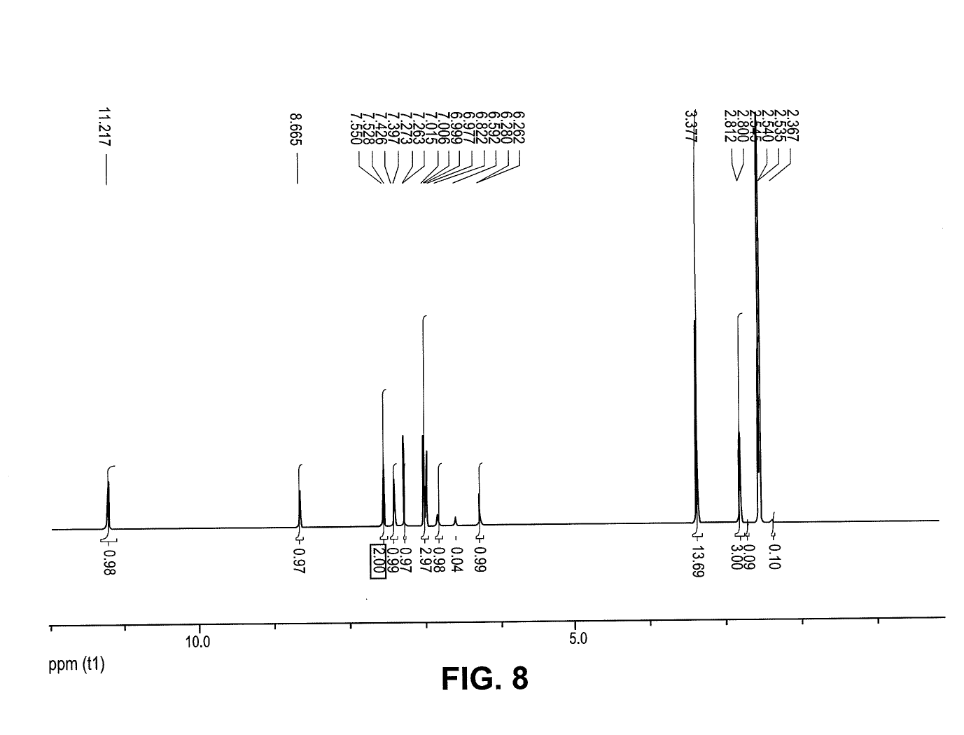

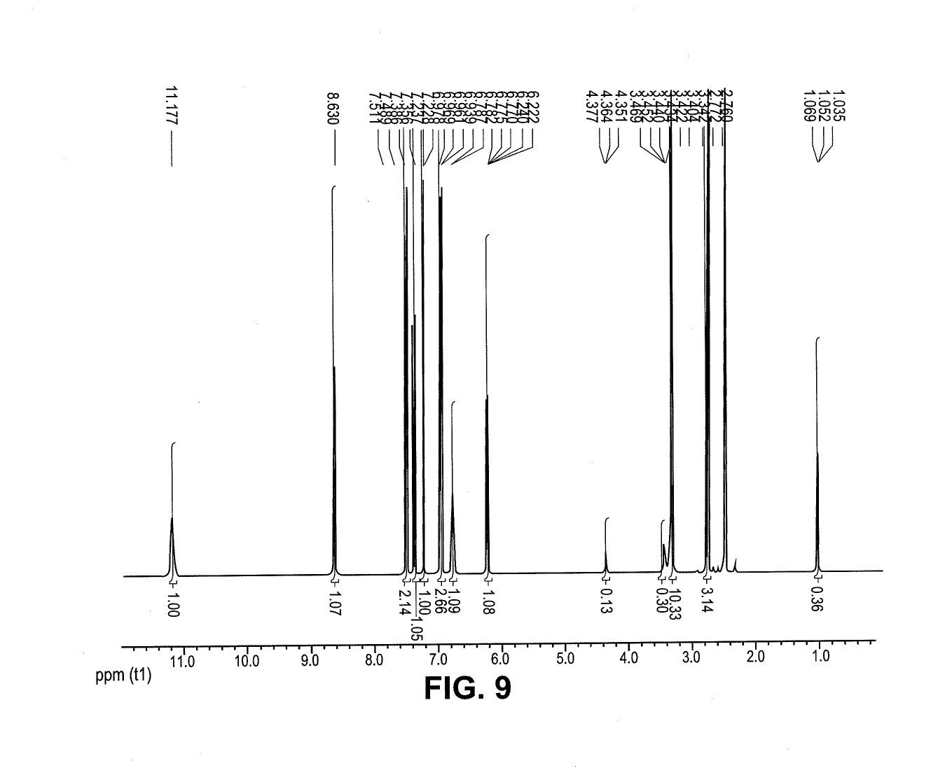

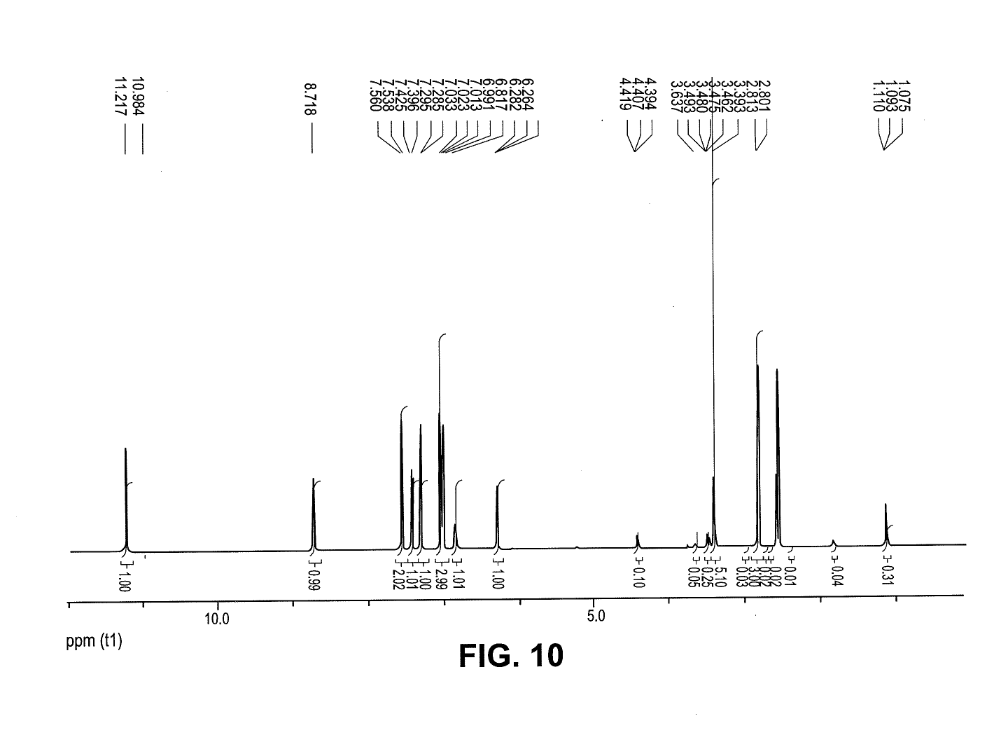

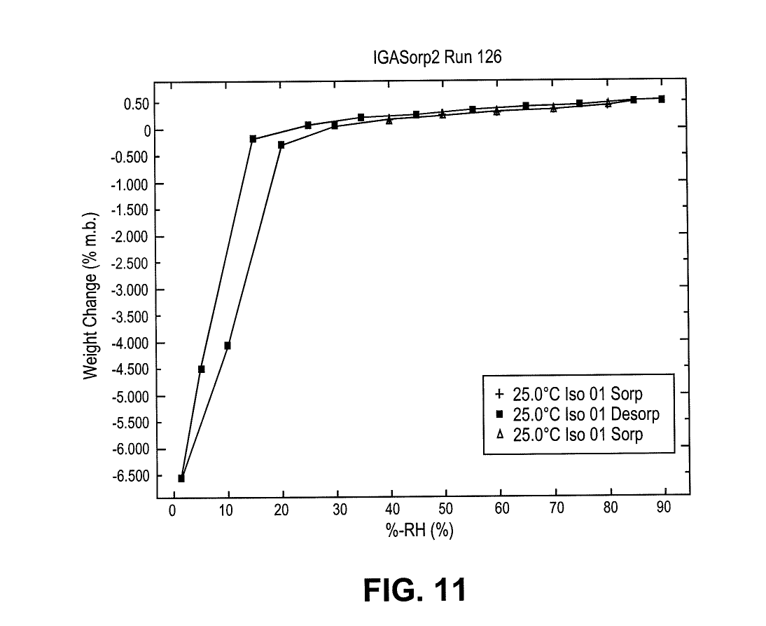

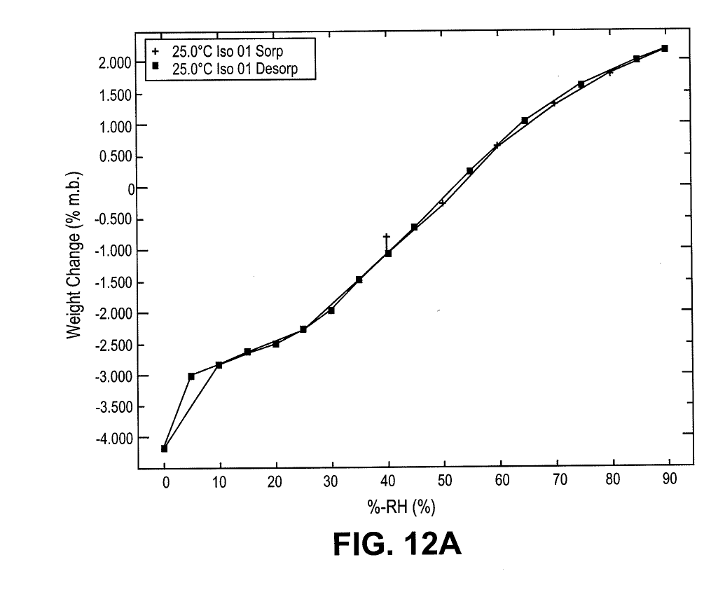

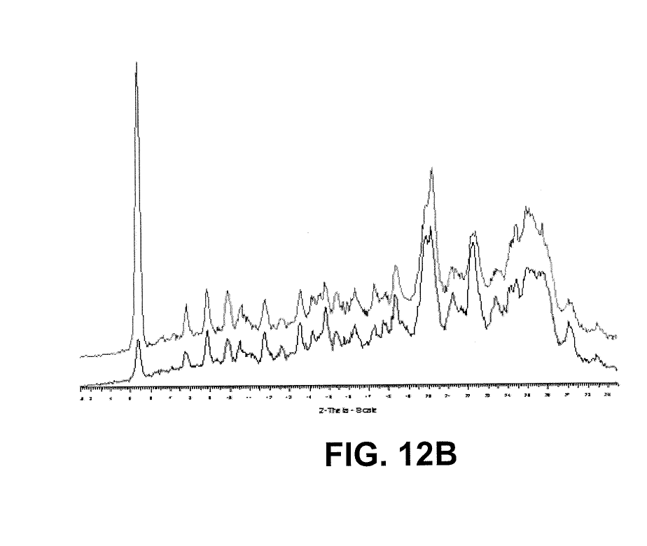

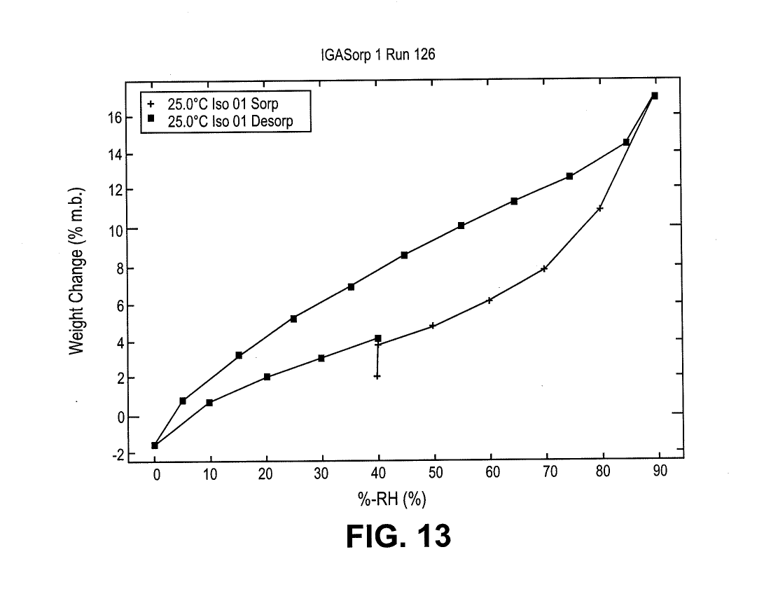

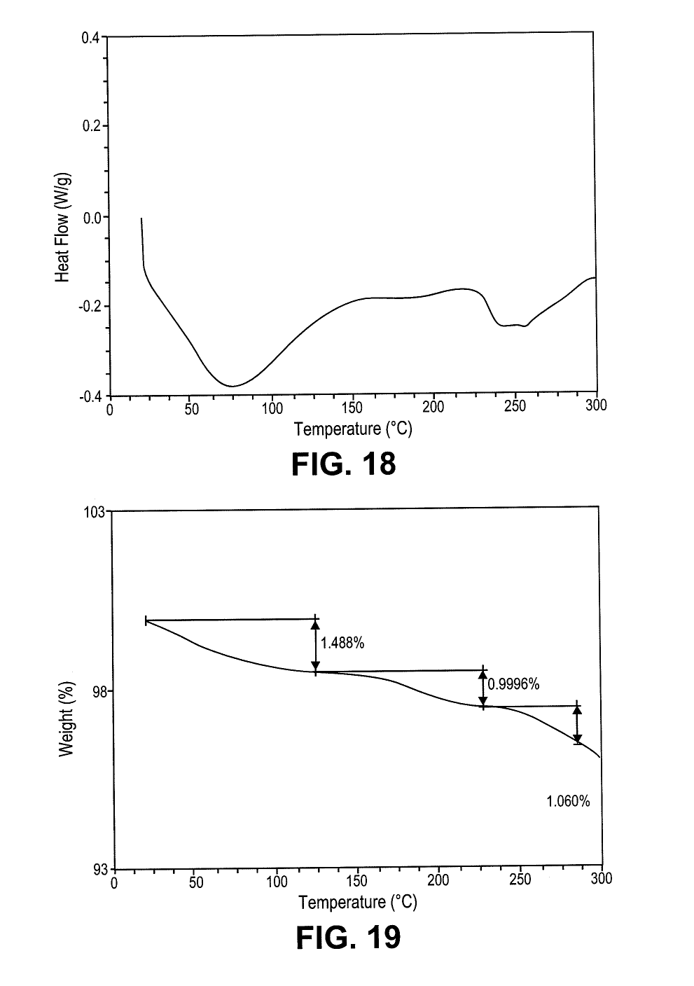

(i) an infra red spectrum in a mineral oil dispersion substantially in accordance with FIG. 7;
(ii) an X-ray powder diffraction pattern substantially in accordance with FIG. 4; and
(iii) a DSC scan substantially in accordance with FIG. 18; herein designated as amorphous form.

In another embodiment, the invention provides for the use of a form of [4-(6-fluoro-7-methylamino-2,4-dioxo-1,4-dihydro-2H-quinazolin-3-yl)-phenyl]-5-chloro-thiophen-2-yl-sulfonylurea sodium salt which provides an infra red spectrum containing absorption peaks at about 3560, 1711, 1632, 1556, 1512, 1445, 1407, 1375, 1309, 1280, 1227, 1133, 1092, 1032, 987, 905, 781, 770 and 691 cm⁻¹; herein designated as amorphous form.

In another embodiment, the invention provides for the use of a crystalline polymorph of [4-(6-fluoro-7-methylamino-2,4-dioxo-1,4-dihydro-2H-quinazolin-3-yl)-phenyl]-5-chloro-thiophen-2-yl-sulfonylurea salts which provides spectrum containing at least one, but fewer than the above peak listings for the designated forms.

Crystalline form A of [4-(6-fluoro-7-methylamino-2,4-dioxo-1,4-dihydro-2H-quinazolin-3-yl)-phenyl]-5-chloro-thiophen-2-yl-sulfonylurea potassium salt is a dihydrate which is stable to 15% relative humidity (RH) at 25° C. but which rehydrates at 20% RH at 25° C. Polymorph A of the potassium salt has been found to be equally stable as the amorphous form of the sodium salt. No change in the chemical purity of either salt form was observed after one week when in accelerated stability tests at high temperature (40° C.) and high relative humidity (75% RH). An advantage of the potassium crystalline form A is that it is less hygroscopic than the amorphous form of the sodium salt which picks up >15% w/w water at 40% RH. Both Form A and B are stable. Form B of the potassium salt is anhydrous and non-hygroscopic (difficult to form a re-hydrated form) Form B of the potassium salt retains a better physical appearance and handling properties over a longer period of time. An improvement in the physical appearance of a dosage form of a drug enhances both physician and patient acceptance and increases the likelihood of success of the treatment.

Further embodiments of the invention include the use of mixtures of the different crystalline solid forms, and the amorphous form, of [4-(6-fluoro-7-methylamino-2,4-dioxo-1,4-dihydro-2H-quinazolin-3-yl)-phenyl]-5-chloro-thiophen-2-yl-sulfonylurea and its salts. Such mixtures include compositions comprising at least one solid form or at least two solid forms selected from Form A, Form B and the amorphous form. Any of the analytical techniques described herein may be used to detect the presence of the solid forms in such compositions. Detection may be done qualitatively, quantitatively, or semi-quantitatively as those terms as used and understood by those of skill in the solid-state analytical arts.

For these analyses, use of standard analytical techniques involving reference standards may be used. Further, such methods may include use of techniques such as partial-lease squares in conjunction with a diffractive or spectroscopic analytical technique. These techniques may also be used in pharmaceutical compositions of the invention.

### V. PREPARATION OF CRYSTALLINE SOLID AND AMORPHOUS FORMS FOR USE ACCORDING TO THE INVENTION

Furthermore, the present invention is directed to the use of crystalline solid and amorphous forms of [4-(6-fluoro-7-methylamino-2,4-dioxo-1,4-dihydro-2H-quinazolin-3-yl)-phenyl]-5-chloro-thiophen-2-yl-sulfonylurea potassium and sodium salts.

Crystalline solid and amorphous forms of the compounds for use according to the invention may be prepared by various methods as outlined below. Other well-known crystallization procedures as well as modification of the procedures outline above may be utilized.

In another embodiment of the present invention, the invention uses [4-(6-fluoro-7-methylamino-2,4-dioxo-1,4-dihydro-2H-quinazolin-3-yl)-phenyl]-5-chloro-thiophen-2-yl-sulfonylurea potassium salt in a crystalline solid form A, which can be obtained by at least one of:

(i) crystallizing [4-(6-fluoro-7-methylamino-2,4-dioxo-1,4-dihydro-2H-quinazolin-3-yl)-phenyl]-5-chloro-thiophen-2-yl-sulfonylurea potassium salt from at least one solvent selected from the group consisting of ethanol, methanol, and combinations thereof and drying such that the crystal contained some solvent; and
(ii) heating [4-(6-fluoro-7-methylamino-2,4-dioxo-1,4-dihydro-2H-quinazolin-3-yl)-phenyl]-5-chloro-thiophen-2-yl-sulfonylurea potassium salt in at least one solvent selected from the group consisting of ethanol, methanol, and combinations thereof; crystallizing at a temperature of from about 50° C. to −10° C. and drying until the crystals contained at least about 0.05% solvent.

In another embodiment of the present invention there is provided use of [4-(6-fluoro-7-methylamino-2,4-dioxo-1,4-dihydro-2H-quinazolin-3-yl)-phenyl]-5-chloro-thiophen-2-yl-sulfonylurea potassium salt in a crystalline solid form B, which can be obtained by at least one of:

(i) heating [4-(6-fluoro-7-methylamino-2,4-dioxo-1,4-dihydro-2H-quinazolin-3-yl)-phenyl]-5-chloro-thiophen-2-yl-sulfonylurea potassium salt in a solvent combination of ethanol and water; crystallizing at a temperature of from about 50° C. to −10° C. and drying until the crystals contain less than 0.05% solvent; and
(ii) crystallizing [4-(6-fluoro-7-methylamino-2,4-dioxo-1,4-dihydro-2H-quinazolin-3-yl)-phenyl]-5-chloro-thiophen-2-yl-sulfonylurea potassium salt from a solvent combination of ethanol and water and drying such that the crystal contained less than 0.05% solvent.

In another embodiment of the present invention there is provided for use of a amorphous crystalline form of [4-(6-fluoro-7-methylamino-2,4-dioxo-1,4-dihydro-2H-quinazolin-3-yl)-phenyl]-5-chloro-thiophen-2-yl-sulfonylurea potassium salt which can be prepared by triturating in isopropanol and drying.

In another embodiment of the present invention there is provided a amorphous crystalline form of [4-(6-fluoro-7-methylamino-2,4-dioxo-1,4-dihydro-2H-quinazolin-3-yl)-phenyl]-5-chloro-thiophen-2-yl-sulfonylurea sodium salt which can be obtained by at least one of: (i) heating [4-(6-fluoro-7-methylamino-2,4-dioxo-1,4-dihydro-2H-quinazolin-3-yl)-phenyl]-5-chloro-thiophen-2-yl-sulfonylurea sodium salt in at least one solvent selected from the group consisting of isopropanol, acetonitrile, ethanol and combinations thereof; and crystallizing at a temperature of from about 50° C. to −10° C.;

(ii) crystallizing [4-(6-fluoro-7-methylamino-2,4-dioxo-1,4-dihydro-2H-quinazolin-3-yl)-phenyl]-5-chloro-thiophen-2-yl-sulfonylurea sodium salt from at least one solvent selected from the group consisting of isopropanol, acetonitrile, ethanol and combinations thereof; and
(iii) heating [4-(6-fluoro-7-methylamino-2,4-dioxo-1,4-dihydro-2H-quinazolin-3-yl)-phenyl]-5-chloro-thiophen-2-yl-sulfonylurea sodium salt in high humidity.

Furthermore, the present invention is directed to the above described processes for the preparation of crystalline solid and amorphous forms of [4-(6-fluoro-7-methylamino-2,4-dioxo-1,4-dihydro-2H-quinazolin-3-yl)-phenyl]-5-chloro-thiophen-2-yl-sulfonylurea potassium and sodium salts.

[4-(6-fluoro-7-methylamino-2,4-dioxo-1,4-dihydro-2H-quinazolin-3-yl)-phenyl]-5-chloro-thiophen-2-yl-sulfonylurea in a crystalline solid or amorphous form may be prepared by various methods as further described below in the Examples. The examples illustrate, but do not limit the scope of the present invention. [4-(6-fluoro-7-methylamino-2,4-dioxo-1,4-dihydro-2H-quinazolin-3-yl)-phenyl]-5-chloro-thiophen-2-yl-sulfonylurea in crystalline solid or amorphous forms may be isolated using typical isolation and purification techniques known in the art, including, for example, chromatographic, recrystallization and other crystallization procedures as well as modification of the procedures outlined above.

### VI. PHARMACEUTICAL COMPOSITIONS

A compound of formula (I) for use according to the invention is formulated into pharmaceutical compositions. Accordingly, the invention also provides a pharmaceutical composition for preventing or treating thrombosis in a mammal, particularly those pathological conditions involving platelet aggregation, containing a therapeutically effective amount of a compound of formula (I) or a pharmaceutically acceptable salt thereof, each as described above, and a pharmaceutically acceptable carrier or agent. Preferably, a pharmaceutical composition of the invention contains a compound of formula (I), or a salt thereof, in an amount effective to inhibit platelet aggregation, more preferably, ADP-dependent aggregation, in a mammal, in particular, a human. Pharmaceutically acceptable carriers or agents include those known in the art and are described below.

Pharmaceutical compositions of the invention may be prepared by mixing the compound of formula (I) with a physiologically acceptable carrier or agent. Pharmaceutical compositions of the invention may further include excipients, stabilizers, diluents and the like and may be provided in sustained release or timed release formulations. Acceptable carriers, agents, excipients, stablilizers, diluents and the like for therapeutic use are well known in the pharmaceutical field, and are described, for example, in Remington's Pharmaceutical Sciences, Mack Publishing Co., ed. A. R. Gennaro (1985). Such materials are nontoxic to the recipients at the dosages and concentrations employed, and include buffers such as phosphate, citrate, acetate and other organic acid salts, antioxidants such as ascorbic acid, low molecular weight (less than about ten residues) peptides such as polyarginine, proteins, such as serum albumin, gelatin, or immunoglobulins, hydrophilic polymers such as polyvinylpyrrolidinone, amino acids such as glycine, glutamic acid, aspartic acid, or arginine, monosaccharides, disaccharides, and other carbohydrates including cellulose or its derivatives, glucose, mannose or dextrins, chelating agents such as EDTA, sugar alcohols such as mannitol or sorbitol, counterions such as sodium and/or nonionic surfactants such as TWEEN, or polyethyleneglycol.

Further embodiments of the invention include pharmaceutical compositions of [4-(6-fluoro-7-methylamino-2,4-dioxo-1,4-dihydro-2H-quinazolin-3-yl)-phenyl]-5-chloro-thiophen-2-yl-sulfonylurea, its salts and forms, including in therapeutically effective amounts of Form A, Form B, and the amorphous form. Said amounts of the at least one of said forms may or may not be in therapeutically effective amounts. Such pharmaceutical compositions may be in the form of a solid oral composition such as a tablet or a capsule or as a dry powder for inhalation.

The pharmaceutical compositions of this invention may be in any orally acceptable dosage form, including capsules, tablets, aqueous suspensions or solutions. In the case of tablets for oral use, carriers that are commonly used include lactose and corn starch. Lubricating agents, such as magnesium stearate, are also typically added. For a capsule form, useful diluents include lactose and dried cornstarch. When aqueous suspensions are required for oral use, the active ingredient is combined with emulsifying and suspending agents. If desired, certain sweetening, flavoring or coloring agents may also be added.

In some embodiments, the pharmaceutical compositions is formulated as direct bolus intravenous preparation for administration to a human subject. The compositions can be provided as a low volume, ready-to-use, bolus injectable, aqueous pharmaceutical composition. The volume can be from 1 to 5 ml, or more preferably, from 0.5 ml to 2 ml. The compositions can also be formulated for intravenous infusion. The pharmaceutical composition may comprise from 1 to 50 mg inclusive of the compound in a sterile aqueous formulation. In some embodiments, a buffering agent(s) is used to provide a physiological pH. Such agents may be any one or more of citrate, malate, formate, succinate, acetate, propionate, histidine, carbonate, phosphate, or MES. The composition is accordingly preferably isotonic with blood and may comprise solutes to adjust the tonicity. Co-solvents include propylene glycol, ethanol, or polyethylene glycol.

### VII. METHODS OF TREATMENT/ADMINISTRATION

A. Preventing and Treating Disease Conditions Characterized by Undesired Thrombosis

Methods for preventing or treating thrombosis in a mammal embraced by the invention administering a therapeutically effective amount of a compound of formula (I) alone or as part of a pharmaceutical composition of the invention as described above to a mammal, in particular, a human. Compounds of formula (I) and pharmaceutical compositions for use according to the invention containing a compound of formula (I) are suitable for use alone or as part of a multi-component treatment regimen for the prevention or treatment of cardiovascular diseases, particularly those related to thrombosis. For example, a compound or pharmaceutical composition of the invention may be used as a drug or therapeutic agent for any thrombosis, particularly a platelet-dependent thrombotic indication, including, but not limited to, acute myocardial infarction, unstable angina, chronic stable angina, transient ischemic attacks, strokes, peripheral vascular disease, preeclampsia/eclampsia, deep venous thrombosis, embolism, disseminated intravascular coagulation and thrombotic cytopenic purpura, thrombotic and restenotic complications following invasive procedures, e.g., angioplasty, carotid endarterectomy, post CABG (coronary artery bypass graft) surgery, vascular graft surgery, stent placements and insertion of endovascular devices and prostheses, and hypercoagulable states related to genetic predisposition or cancers. In other groups of embodiments, the indication is selected from the group consisting of percutaneous coronary intervention (PCI) including angioplasty and/or stent, acute myocardial infarction (AMI), unstable angina (USA), coronary artery disease (CAD), transient ischemic attacks (TIA), stroke, peripheral vascular disease (PVD), Surgeries-coronary bypass, carotid endarterectomy.

Compounds and pharmaceutical compositions of the invention may also be used as part of a multi-component treatment regimen in combination with other therapeutic or diagnostic agents in the prevention or treatment of thrombosis in a mammal. In certain preferred embodiments, compounds or pharmaceutical compositions of the invention may be coadministered along with other compounds typically prescribed for these conditions according to generally accepted medical practice such as anticoagulant agents, thrombolytic agents, or other antithrombotics, including platelet aggregation inhibitors, tissue plasminogen activators, urokinase, prourokinase, streptokinase, heparin, aspirin, or warfarin or anti-inflammatories (non-steriodal anti-inflammatories, cyclooxygenase II inhibitors), thrombin inhibitors or Factor Xa inhibitors. Coadministration may also allow for application of reduced doses of both the anti-platelet and the thrombolytic agents and therefore minimize potential hemorrhagic side-effects. Compounds and pharmaceutical compositions of the invention may also act in a synergistic fashion to prevent reocclusion following a successful thrombolytic therapy and/or reduce the time to reperfusion.

Compounds and pharmaceutical compositions of the invention may be in the form of solutions or suspensions. In the management of thrombotic disorders the compounds or pharmaceutical compositions of the invention may also be in such forms as, for example, tablets, capsules or elixirs for oral administration, sterile solutions or suspensions or injectable administration, and the like, or incorporated into shaped articles.

### VIII. EXAMPLES

The Examples are intended to exemplify and not limit the invention.

### Chemistry General Methods

The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis, Wiley & Sons: New York, 1967-2004, Volumes 1-22; Rodd's Chemistry of Carbon Compounds, Elsevier Science Publishers, 1989, Volumes 1-5 and Supplementals; and Organic Reactions, Wiley & Sons: New York, 2005, Volumes 1-65. The following synthetic reaction schemes are merely illustrative of some methods by which the compounds for use according to the present invention can be synthesized, and various modifications to these synthetic reaction schemes can be made and will be suggested to one skilled in the art having referred to the disclosure contained in this Application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein preferably are conducted under an inert atmosphere at atmospheric pressure at a reaction temperature range of from about −78° C. to about 150° C., more preferably from about 0° C. to about 125° C., and most preferably and conveniently at about room (or ambient) temperature, e.g., about 20° C. to about 75° C.

Referring to the examples that follow, compounds for use according to the present invention were synthesized using the methods described herein, or other methods, which are well known in the art.

The compounds and/or intermediates were characterized by high performance liquid chromatography (HPLC) using a Waters Alliance chromatography system with a 2695 Separation Module (Milford, Mass.). The analytical columns were C-18 SpeedROD RP-18E Columns from Merck KGaA (Darmstadt, Germany). Alternately, characterization was performed using a Waters Unity (HPLC) system with Waters Acquity HPLC BEH C-18 2.1 mm×15 mm columns. A gradient elution was used, typically starting with 5% acetonitrile/95% water and progressing to 95% acetonitrile over a period of 5 minutes for the Alliance system and 1 minute for the Acquity system. All solvents contained 0.1% trifluoroacetic acid (TFA). Compounds were detected by ultraviolet light (UV) absorption at either 220 or 254 nm. HPLC solvents were from EMD Chemicals, Inc. (Gibbstown, N.J.). In some instances, purity was assessed by thin layer chromatography (TLC) using glass backed silica gel plates, such as, for example, EMD Silica Gel 60 2.5 cm×7.5 cm plates. TLC results were readily detected visually under ultraviolet light, or by employing well known iodine vapor and other various staining techniques.

Mass spectrometric analysis was performed on one of two Agilent 1100 series LCMS instruments with acetonitrile/water as the mobile phase. One system using TFA as the modifier and measures in positive ion mode [reported as MH+, (M+1) or (M+H)+] and the other uses either formic acid or ammonium acetate and measures in both positive [reported as MH⁺, (M+1) or (M+H)⁺] and negative [reported as M−, (M−1) or (M−H)⁻] ion modes.

Nuclear magnetic resonance (NMR) analysis was performed on some of the compounds with a Varian 400 MHz NMR (Palo Alto, Calif.). The spectral reference was either TMS or the known chemical shift of the solvent.

The purity of some of the invention compounds is assessed by elemental analysis (Robertson Microlit, Madison N.J.).

Melting points are determined on a Laboratory Devices MeI-Temp apparatus (Holliston, Mass.).

Preparative separations were carried out using either an Sq16x or an Sg100c chromatography system and prepackaged silica gel columns all purchased from Teledyne Isco, (Lincoln, Nebr.). Alternately, compounds and intermediates were purified by flash column chromatography using silica gel (230-400 mesh) packing material, or by HPLC using a C-18 reversed phase column. Typical solvents employed for the Isco systems and flash column chromatography were dichloromethane, methanol, ethyl acetate, hexane, acetone, aqueous hydroxyamine and triethyl amine. Typical solvents employed for the reverse phase HPLC were varying concentrations of acetonitrile and water with 0.1% trifluoroacetic acid.

### Instrumental for Solid Forms

### 1. FT Infrared Spectroscopy (FTIR)

Samples were studied on a Perkin-Elmer Spectrum One fitted with a Universal ATR sampling accessory and running Spectrum V5.0.1 software. The resolution was set to 4 cm-1 and 16 scans were collected over the range 4000 cm⁻¹ to 400 cm⁻¹. Control and Analysis software: Spectrum v 5.0.1.

### 2. Differential Scanning Calorimetry (DSC)

DSC data (thermograms) were collected on a TA instruments Q1000 equipped with a 50 position auto-sampler. The energy and temperature calibration standard was indium.

Samples were heated at a rate of 10° C./min from 10° C. to 250° C. A nitrogen purge at 30 ml/min was maintained over the sample.

Between 1 and 3 mg of sample was used, unless otherwise stated, and all samples were sealed in an aluminum pan with a pinhole in the lid. Control software: Advantage for Q series v 2.2.0.248, Thermal Advantage Release 4.2.1. Analysis software: Universal Analysis 2000 v 4.1D Build 4.1.0.16

### 3. Thermogravimetric Analysis (TGA)

TGA data (thermograms) were collected on a TA Instrument Q500 TGA with a 16 position auto-sampler. Samples were heated at a rate of 10° C./minute. A nitrogen purge of 100 ml/min was maintained over the sample.

Typically 5-20 mg of sample was loaded onto a tared open aluminum open pan. Control software: Advantage for Q series v 2.2.0.248, Thermal Advantage Release 4.2.1. Analysis software: Universal Analysis 2000 v 4.1 D Build 4.1.0.16

### 4. XRPD (X-Ray Powder Diffraction)

### Bruker AXS C2 GADDS Diffractometer

X-ray powder diffraction patterns for the samples were acquired on a Bruker AXS C2 GADDS diffractometer using Cu Kα radiation (40 kV, 40 mA), automated XYZ stage, laser video microscope for auto-sample positioning and a HiStar 2-dimensional area detector. X-ray optics consists of a single Göbel multilayer mirror coupled with a pinhole collimator of 0.3 mm.

Beam divergence, i.e. the effective size of the X-ray beam on the sample, was approximately 4 mm. A 0-0 continuous scan mode was employed with a sample to detector distance of 20 cm which gives an effective 2θ range of 3.2°-29.8°. A typical exposure time of a sample was 120s.

Samples run under ambient conditions were prepared as flat plate specimens using powder as received without grinding. Approximately 1-2 mg of the sample was lightly pressed on a glass slide to obtain a flat surface. Control software: GADDS for WNT v 4.1.16. Analysis software: Diffrac Plus Release 3 EVA v 9.0.0.2

### 5. Gravimetric Vapor Sorption (GVS) Studies

Isotherms were collected on a Hiden IGASorp moisture sorption analyzer running CFRSorp software. Sample sizes were typically ca. 10 mg. A moisture adsorption/desorption isotherm was performed as outlined below. The samples were loaded and unloaded at room humidity and temperature (ca. 40% RH, 25° C.). The standard isotherm run was a single cycle starting at 40% RH. The humidity was stepped as follows: 40, 50, 60, 70, 80, 90, 85, 75, 65, 55, 45, 35, 25, 15, 5, 0, 10, 20, 30, 40. Control and Analysis software: IGASorp Controller v 1.10, IGASorp Systems Software v 3.00.23.

### 6. ¹H NMR

Spectra were collected on a Bruker 400 MHz equipped with auto sampler. Samples were prepared in d₆-DMSO.

### 7. Purity Analysis

Purity analysis was performed on an Agilent HP1100 system equipped with a diode array detector.

### Method: Gradient

Column details: Betabasic C18, 5 μm, 150×4.6 mm

### Column Temperature: 25° C.

Injection volume: 5 μl
Flow Rate ml/min: 0.8 ml/min
Detection wavelength: 325 nm
Phase A: 0.1% v/v aqueous formic acid
Phase B: Acetonitrile: water 90:10 with 0.1% v/v formic acid

TABLE 3
Mobile phase timetable.

| Time/Min | % A | % B |
|---|---|---|
| 0 | 90 | 10 |
| 2 | 90 | 10 |
| 17 | 10 | 90 |
| 21 | 10 | 90 |
| 21.3 | 90 | 10 |
| 25 | 90 | 10 |

TABLE 4

| | potassium salt | sodium salt |
|---|---|---|
| Purity | 99.4% (a/a) | 99.4% (a/a) |
| Impurities | | |
| Individual peaks ≧ | % (a/a) | % (a/a) |
| 0.1% (a/a) | | |
| RRT = 0.57 | 0.14 | 0.11 |
| RRT = 1.08 | 0.15 | 0.18 |
| Total of peaks <0.1% (a/a) | 0.3 | 0.3 |

### Example 1

### Synthesis of the Intermediate Sulfonylurea Carbamate (8)

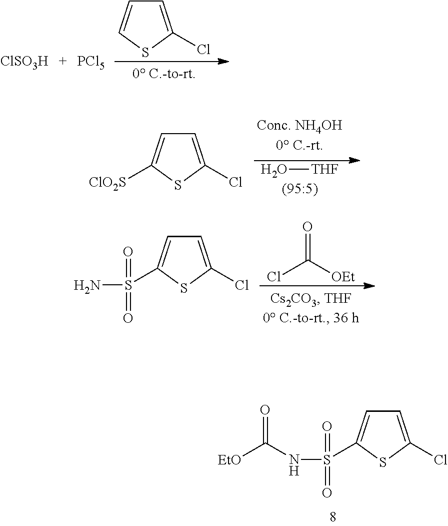

### Step 1—Preparation 5-chlorothiophene-2-sulfonyl chloride

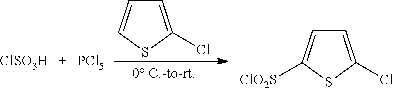

The following procedure was adapted from C. A. Hunt, et al. J. Med. Chem. 1994, 37, 240-247. In a three-necked R.B. flask, equipped with a mechanical stirrer, an air condenser, a dropping funnel, and a moisture-guard tube, was placed chlorosulfonic acid (240 mL, 3.594 mol). Under stirring, PCl₅ (300 g, 1.44 mol, 0.40 equiv) was added in portions, over ca. 45 mins. During the addition, a large volume of HCl gas evolved vigorously, but the temperature of the mixture did not rise significantly (<40° C.). By the time all the PCl₅ had been added, an almost clear, pale yellow solution resulted, with only a few solid pieces of PCl₅ floating in the suspension. It was stirred until gas evolution ceased (0.5 h).

Then the reaction vessel was cooled in ice, and 2-chloro-thiophene (66.0 mL, 0.715 mol) was added via the dropping funnel, over 1.0 h. With the addition of the very first few drops of 2-Cl-thiophene, the mixture turned dark purple, and by the time all of the thiophene had been added, a dark purple solution resulted. During the addition, HCl gas evolved continuously, at a slow rate. The reaction mixture was then stirred at room temperature overnight.

Then the mixture, dark-purple clear solution, was added dropwise to crushed ice (3 L), over 0.5 h. On addition to ice, the purple color disappeared instantaneously; the colorless thin emulsion was stirred mechanically at room temperature for ca. 15 h. Then the mixture was extracted with CH₂Cl₂ (3×300 mL). The combined CH₂Cl₂-extract was washed with water (1×200 mL), saturated NaHCO₃ (1×250 mL), brine (1×100 mL), dried (Na₂SO₄), and concentrated on a rotary evaporator to yield the crude product as a pale yellow glue, which showed a tendency to solidify, yielding a semi-solid mass. This was then purified by high-vacuum distillation (bp 110-112°/12 mm) to yield 135.20 g (88%) of the title compound as a colorless/pale-yellow semi solid.

### Step 2—5-chlorothiophene-2-sulfonamide

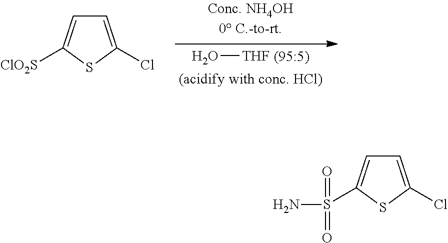

The following procedure was adapted from C. A. Hunt, et al. J. Med. Chem. 1994, 37, 240-247. In a three-necked R. B. flask, equipped with a mechanical stirrer, conc. NH₄OH (500 mL, 148.50 g NH₃, 8.735 mol NH₃, 13.07 equiv NH₃) was placed. The flask was cooled in ice and 5-chlorothiophene-2-sulfonyl chloride (145.0 g, 0.668 mol) was added, in portions over 0.5 h (it is a low-melting solid, and it was melted by warming, which was then conveniently added via a wide-bored polyethylene pipette). The sulfonyl chloride immediately solidifies in the reaction flask. After all the sulfonyl chloride had been added, the flask containing it was rinsed with THF (25 mL), and this also was transferred to the reaction vessel. Then the heavy suspension was stirred at room temperature for ca. 20 h. At the end of this time the reaction mixture was still a suspension but of a different texture.

Then the mixture was cooled in ice, diluted with H₂O (1.5 l), and acidified with conc. HCl to pH ca. 3. The solid product was collected by filtration using a Buchner funnel, rinsed with cold water, and air-dried to afford the title compound as a colorless solid, 103.0 g (78%). MS (M−H): 196.0; 198.0.

### Step 3—Ethyl 5-chlorothiophen-2-ylsulfonylcarbamate

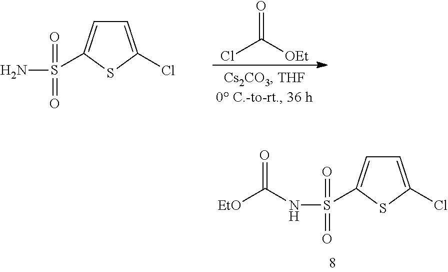

A 2-L 3-necked R.B. flask, equipped with a mechanical stirrer and a dropping funnel, was charged with sulfonamide (60.0 g, 303.79 mmol), and Cs₂CO₃ (200 g, 613.83 mmol, 2.02 equiv) in THF (900 mL). The clear solution was cooled in ice, and ethyl chloroformate (70.0 mL, 734.70 mmol, 2.418 equiv) was added over ca. 30 mins. The heavy suspension was then stirred at room temperature for ca. 36 h.

Then the mixture was diluted with water (200 mL) to yield a clear colorless solution, which was concentrated on rotary evaporator to one-third its volume. This was then diluted with EtOAc (250 mL), cooled in ice, and acidified with 6N HCl to pH ca. 1. The biphasic mixture was transferred to a separatory funnel, layers were separated, and the aqueous layer was again extracted with 2×75 mL EtOAc. The combined organic extract was washed with water/brine (2×50 mL), brine (1×50 mL), dried over Na₂SO₄, and concentrated to yield the title compound as lightly colored oil. This was purified by filtration through a silica-gel plug. The crude product was applied to the silica-gel plug on a sintered funnel in EtOAc, and then was eluted with EtOAc (1 liter). Concentration of the EtOAc filtrate provided the title compound 8 as a colorless solid, 71.28 g (87%). MS (M−H): 268.0; 270.0. ¹H NMR (DMSO): δ 7.62 (d, 1H), 7.25 (d, 1H), 4.10 (q, 2H), 1.16 (t, 3H).

### Example 2

### Synthesis of [4-(6-fluoro-7-methylamino-2,4-dioxo-1,4-dihydro-2H-quinazolin-3-yl)-phenyl]-5-chloro-thiophen-2-yl-sulfonylurea (7a)

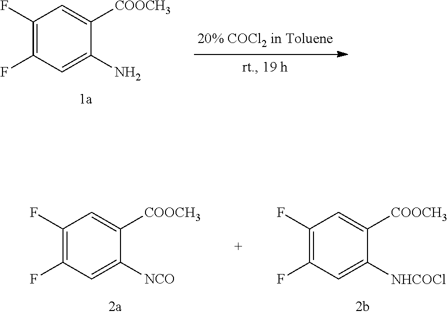

### Step 1

Aniline 1 (¹H NMR (DMSO): δ 7.58 (dd, 1H), 6.72 (dd, 1H), 3.77 (s, 3H); 6.0 g, 32.085 mmol) was placed in a 500 mL round bottomed flask and 20% phosgene in toluene (175 mL, 332.50 mmol, 10.36 equiv) was added. The resulting somewhat sticky suspension was then magnetically stirred overnight at room temperature resulting in a clear, colorless solution. An aliquot removed, blown dry with argon, quenched with MeOH, and analyzed by RP-HPLC/MS to show no unreacted aniline 1 and clean formation of the isocyanate 2a and/or carbamoyl chloride 2b as analyzed as its methyl-carbamate. The mixture was concentrated first by rotary evaporation and then under high vacuum to yield 6.76 g (99% yield) of the isocyanate 2a and/or carbamoyl chloride 2b as a free-flowing colorless solid.

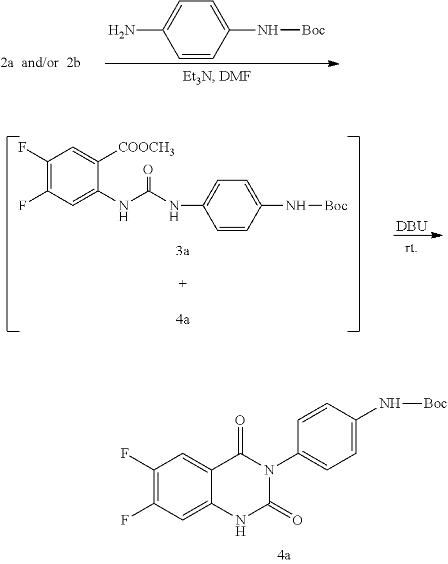

### Step 2

In a 500 mL R. B. flask was placed N-Boc-1,4-phenylenediamine (6.22 g, 29.866 mmol, 1.20 equiv) in DMF (100 mL). Triethylamine (5.30 mL, 38.025 mmol, 1.52 equiv) was syringed in. Then the clear, dark-brown solution was treated with a solution of the isocyanate 2a (5.30 g, 24.88 mmol) and/or carbamoyl chloride 2b in DMF (50 mL), dropwise, over 15 minutes. After the addition was over, a slightly turbid mixture resulted, which was stirred overnight at room-temperature. An aliquot was analyzed, after quenching with MeOH, to show no unreacted isocyanate, and clean formation of the urea, 3a, and quinazoline-1,3-dione, 4a, in a ratio of ca. 2.5:1. MS (M−H): 388.0.

DBU (3.75 mL, 25.07 mmol, ca. 1.0 equiv) was then syringed in, dropwise, over 5 minutes, resulting in a clear dark-brown solution. This was stirred at room temperature for 3.0 h resulting in a turbid mixture. HPLC analysis showed no urea 3a and clean formation of the quinazoline-1,3-dione 4a. The reaction mixture was concentrated on a rotary evaporator to yield the crude product as a solid. This was dried under high vacuum, and then triturated with CH₂Cl₂/H₂O (5:1) to yield 8.40 g of 4a as an almost colorless solid (87% yield). ¹H NMR (DMSO): δ 9.39 (s, 1H), 7.68 (dd, 1H), 7.45 (d, 2H), 7.03 (m, 2H), 6.98 (dd, 1H), 1.48 (s, 9H).

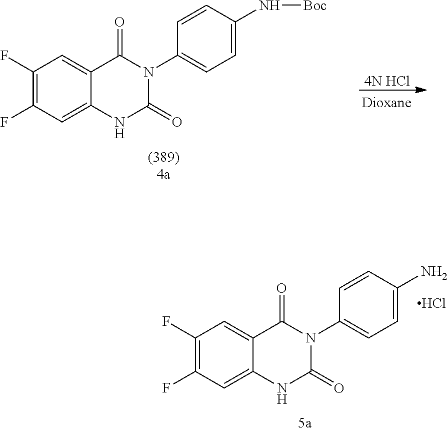

### Step 3

The N-Boc-aniline 4a (4.0 g, 10.28 mmol) was placed in a round-bottomed. flask and 4N HCl in dioxane (50.0 mL, 200 mmol, 19.40 equiv) was added. The heavy, negligibly solvated suspension was stirred at room temperature for 5.0 h. HPLC showed no starting material and clean formation of the aniline 5a. The mixture was then concentrated on a rotary evaporator to yield the crude product. The solid thus obtained was triturated with CH₂Cl₂ to yield 3.22 g of pure 5a as an almost colorless solid (96% yield). MS (M−H): 290.3. ¹H NMR (DMSO): δ 11.75 (s, 1H), 7.88 (dd, 1H), 7.32 (m, 4H), 7.21 (dd, 1H).

### Step 4

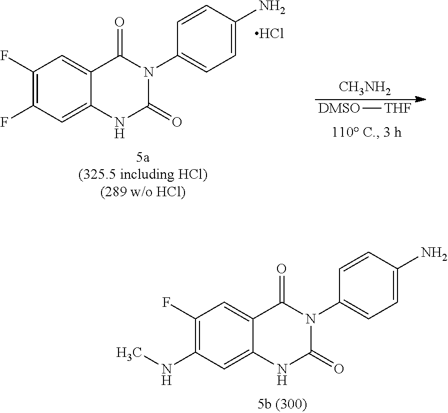

The difluoro-compound, 5a (1.0 g, 3.072 mmol) was placed in a screw-cap sealed tube. DMSO (20 mL) was added, followed by methylamine (2.0M in THF) (15.0 mL, 30 mmol, 9.76 equiv), resulting in a clear solution. This was then heated in an oil bath to 110° C. for 3 h. HPLC showed no unreacted 5a and clean formation of 5b. The mixture was then cooled to room temperature, all the MeNH₂ and THF were evaporated, and the residue was diluted with 100 mL water to precipitate 5b. After stirring for ca. 2 h at room temperature, the colorless solid was collected by filtration through a Buchner funnel and rinsed with H₂O (100 mL), and air-dried. HPLC analysis of this solid showed it to be pure and devoid of any DBU. This solid was further purified by triturating with Et₂O, and then CH₂Cl₂ as in the previous route to this aniline to give 875 mg of the title compound (95% yield). MS (M+1) 301.2. ¹H NMR (DMSO): δ 11.10 (s, 1H), 7.36 (d, 1H), 6.78 (d, 2H), 6.75 (m, 1H), 6.56 (d, 2H), 6.20 (d, 1H), 5.18 (d, 2H), 2.76 (d, 3H).

### Step 5—Synthesis of 1-(5-chlorothiophen-2-ylsulfonyl)-3-(4-(6-fluoro-7-(methylamino)-2,4-dioxo-1,2-dihydroquinazolin-3(4H)-yl)phenyl)urea (7a)

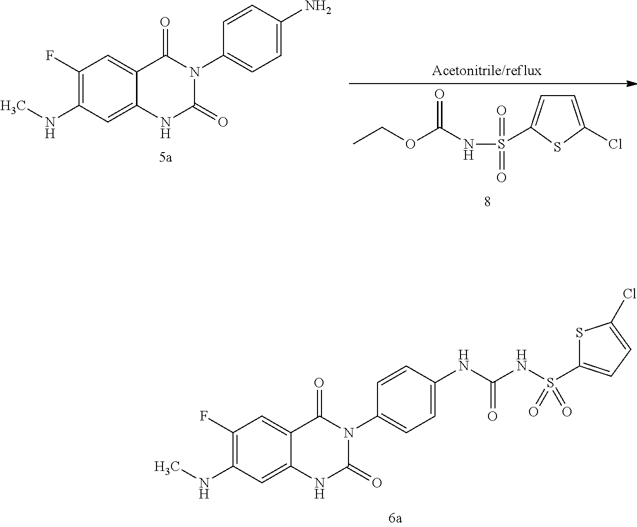

The reaction mixture comprising of the aniline (16.0 g, 53.33 mmol) and ethyl-sulfonyl-carbamate (28.77 g, 106.66 mmol, 2.0 equiv) in CH₃CN (1300 mL) was heated to reflux for 36 h. During this time, the reaction mixture remained as a heavy suspension. HPLC analysis showed a clean reaction, and <1% unreacted anilne. The heavy suspension was cooled to room temperature and filtered through a Buchner funnel. The colorless solid product was further rinsed with CH₃CN (3×40 mL). HPLC of the filtrate showed the presence of only a trace amount of the desired product, most of it being the excess carbamate. The crude product was then triturated with CH₂Cl₂ (400 mL), and the almost colorless solid product was collected by filtration through a Buchner funnel: Yield, 25.69 g (92%). MS (M+1): 524.0; 526.0. ¹H NMR (DMSO):

δ 11.20 (s, 1H), 9.15 (s, 1H), 7.68 (d, 1H), 7.42 (d, 2H), 7.36 (d, 1H), 7.26 (m, 1H), 7.16 (d, 2H), 6.78 (m, 1H), 6.24 (d, 1H), 2.78 (d, 3H).

### Example 3

### Synthesis of [4-(6-fluoro-7-methylamino-2,4-dioxo-1,4-dihydro-2H-quinazolin-3-yl)-phenyl]-5-chloro-thiophen-2-yl-sulfonylurea (6a) and salt (7a)

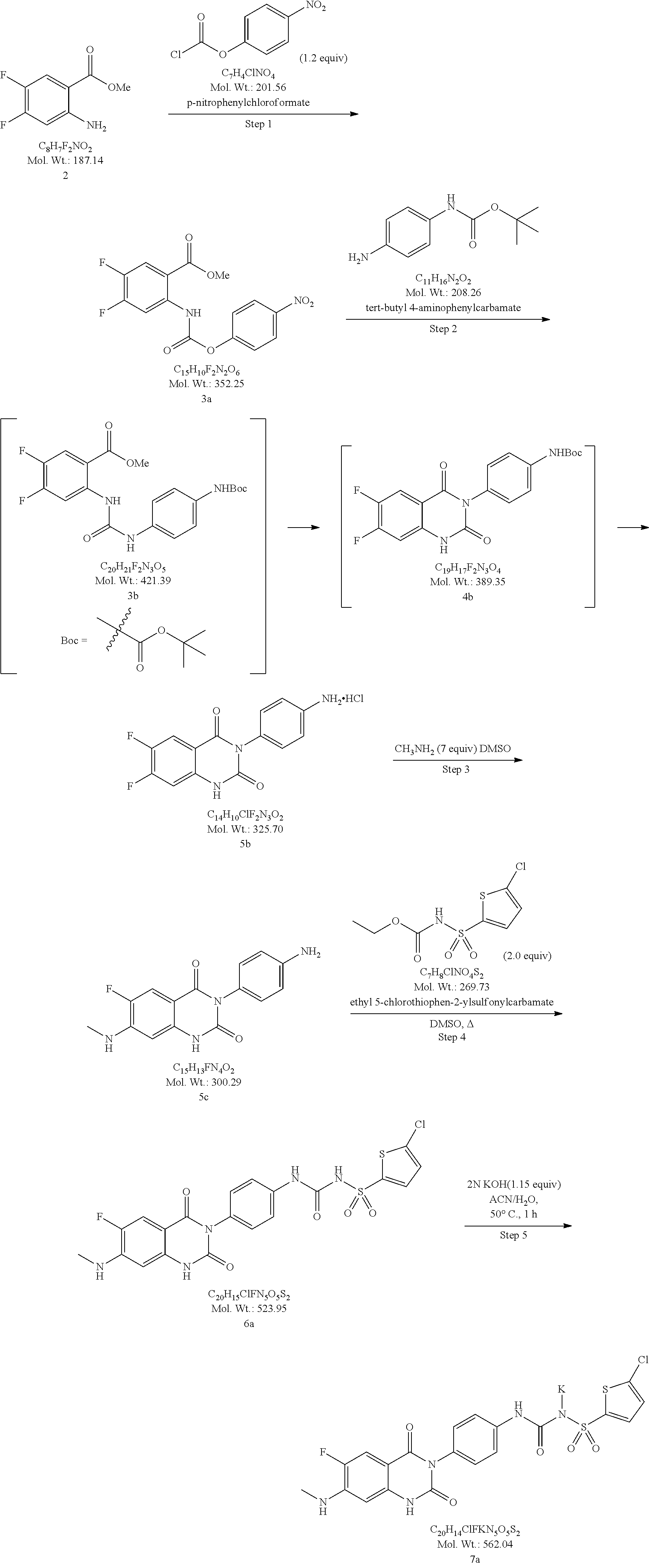

### Step 1

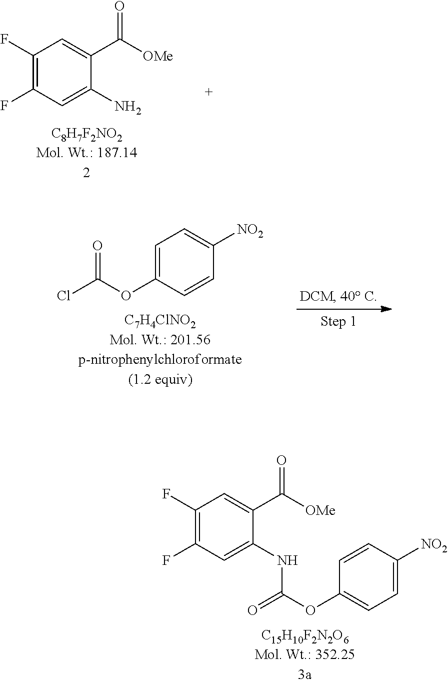

Methyl 2-amino-4,5-difluorobenzoate [2] (38 Kg, 1.0 eq) and dichloromethane (560 Kg, 8×, ACS >99.5%) were charged to a PP1-R1000 reactor (2000 L GL reactor). The reaction mixture was agitated for 5 mins. 4-Nitrophenylchloroformate (49.1 Kg, 1.2 equiv) was charged into PP1-R2000 reactor (200 L) followed by dichloromethane (185 Kg) and agitated the contents for 5 mins. After pressurizing the 200 L reactor the 4-nitrophenylchloroformate solution was transferred into the 2000 L reactor containing dichloromethane solution of [2]. The reaction mixture was heated to 40±5° C. (reflux) under nitrogen gas purge for 3 hrs. The representative TLC analysis confirmed reaction completion (in-process TLC, no compound 2 remaining; 99:1 CHCl₃-MeOH). The solution was cooled to 30° C. and distilled off 460 Kg of dichloromethane under vacuum. The 2000 L reactor was charged with 520 Kg of hexanes and cooled the contents of the reactor to 0±5° C. and agitated for 4 hrs. The solid obtained was filtered through GF Nutsche filter lined with a sheet of T-515 LF Typar filter and a sheet of MeI-Tuf 1149-12 filter paper. The filter cake was washed with 20 Kg of hexanes and vacuum dried at 35° C. until constant weight attained. The dry product was discharged (70.15 Kg) with 98% yield. The product confirmed by ¹H NMR and TLC analysis.

### Step 2. Synthesis of 3-(4-aminophenyl)-6,7-difluoroquinazoline-2,4(1H, 3H)-dione hydrochloride, compound 5b

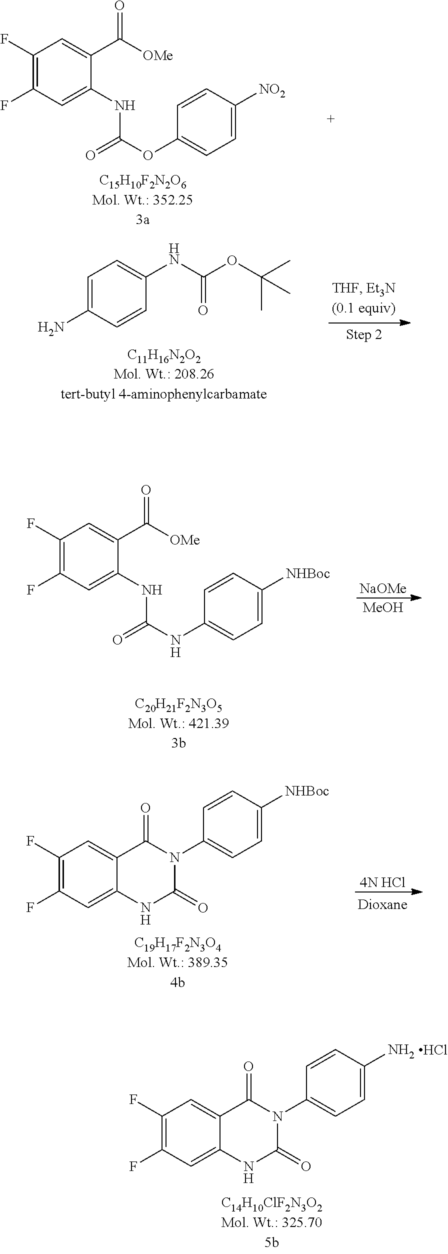

The PP1-R1000 (2000 L GL reactor) reactor was charged with 3a (64.4 Kg, 1.0 eq), anhydrous tetrahydrofuran (557 Kg) and triethylamine (2.2 Kg, 0.1 equiv). The charging line of 2000 L GL reactor was rinsed with tetrahydrofuran (10 Kg). The contents of the reactor were agitated for 25 mins. during that period complete solution was obtained. The PP1-R2000 (200 L HP reactor) reactor was charged with N-Boc-p-phenylenediamine (38 Kg, 1.0 equiv), tetrahydrofuran (89 Kg) and agitated for 30 mins. until complete solution obtained. The contents of the 200 L HP reactor were transferred to the 2000 L GL reactor containing the compound 3a and then heated at 65±5° C. for 2 hrs. The reaction was deemed complete monitored by HPLC after confirming the disappearance of starting material 3a (in-process specification <1%). The contents of 2000 L GL reactor were cooled to 20±5° C. and then charged with sodium methoxide (25% solution in methanol, 41.5 Kg, 1.05 equiv.) over 20 mins. maintaining the temperature below 30° C. The charging lines were rinsed with tetrahydrofuran (10 Kg). The contents were agitated at 25±5° C. for 4 hrs. In-process HPLC analysis confirmed the completion of the reaction when the amount of compound 3b remaining in the reaction mixture is <1%. To this reaction mixture added filtered process water (500 Kg) and distilled under vacuum the 2000 L GL reactor contents into clean 200 L GL receiver until 300 Kg of solvent is distilled. The solids obtained were filtered using GL Nutsche filter and washed with process filtered water until the color of the solid the compound 4b is white to grayish. The 2000 L GL reactor is charged with wet compound 4b filter cake, dioxane (340 Kg) and agitated the contents for 1 hr. The filterable solid obtained were filtered through GL Nutsche filter with a sheet of T-515 LF Typar filter paper. The solid cake was blow dried for 2 hrs and then charged with dioxane (200 Kg) into the 2000 L GL reactor. The contents were agitated for 10 min. and then charged with 4 N HCl in dioxane (914 Kg) over 3 hrs and maintaining the internal temperature below 30° C. The charging line was rinsed with additional dioxane (10 Kg) and the contents of the reactor were agitated for 6 hrs at 25±5° C. The completion of the reaction is monitored by HPLC (in process control compound 4 is <1% in the reaction mixture) for the conversion of compound 4b to compound 5b. The contents of the reactor were cooled to 5+5° C. for 2 hr and the solid obtained was filtered through GL Nutsche filter followed by washing with dioxane (50 Kg). The filter cake was blow dried with 8±7 psig of nitrogen for 30 mins. and purity analyzed by HPLC. The filtered solid was dried to constant weight in vacuum oven at 45° C. for 48 hr. The compound 5b (65.8 Kg, actual yield 110.6%) was discharged and analyzed by ¹HNMR and HPLC analysis. ¹H NMR (DMSO): δ 11.75 (s, 1H), 7.88 (dd, 1H), 7.32 (m, 4H), 7.21 (dd, 1H).

### Step 3. Synthesis of 3-(4-aminophenyl)-6-fluoro-7-(methylamino)quinazoline-2,4(1H, 3H)-dione, Compound 5c

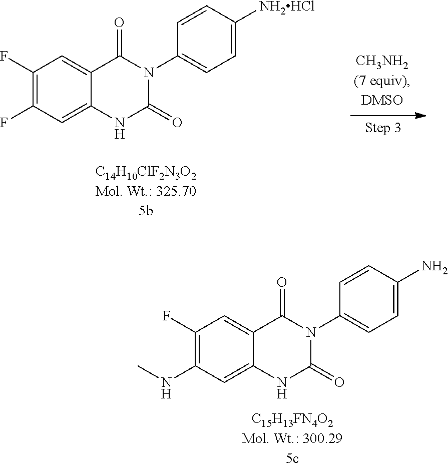

The PP1-R2000 (200 L HP reactor) was charged with compound 5b (18 Kg, 1.0 eq.) and pressurized with 100±5 psig of nitrogen. Vent the nitrogen from the reactor through the atmospheric vent line then open the condenser valve and then charged dimethyl sulfoxide into the reactor (>99.7%, 105 Kg) under blanket of argon. The reactor contents were agitated at 22° C. (19-25° C.) for 15 mins. and then pulled maximum achievable vacuum on the 200 L HP reactor and close all the valves. Using the established vacuum charged to the 200 L HP reactor methylamine (33% wt % in absolute ethanol, 37.2 Kg) at a rate that maintains the internal temperature at 25±5° C. and kept a nitrogen blanket on the reagent solution during charging. After rinsing the charging line with dimethyl sulfoxide (5 Kg) closed the 200 L HP reactor condenser valve and heated the reactor contents to 110±5° C. The contents of the reactor were agitated for at least 5 hrs. at 110±5° C. In-process HPLC taken after 5 hr 40 mins. showed compound 5b content of 0.09%, indicating completion of the reaction (in-process specification ≦1%). The contents of 200 L HP reactor were cooled to 25±5° C. While the 200 L reactor is cooling, closed all the valves of the PP1-R1000 reactor (2000 L GL reactor) and charged with process filtered water (550 Kg). The contents of the 200 L HP reactor were transferred to the 2000 L GL reactor over 15 minutes followed by rinsing the charging line with process filtered water (50 Kg). The contents of the 2000 L GL reactor were agitated for 2 hrs at 5±5° C. The filterable solids obtained were filtered onto PPF200 (GL nutsche filter) fitted with MeI-Tuf 1149-12 filter paper under vacuum. The wet filter cake was discharged and transferred into pre-lined vacuum trays with Dupont's fluorocarbon film (Kind 100A). Clamped down the special oven paper (KAVON 992) over the vacuum trays containing the wet compound 6 and transferred to the vacuum oven tray dryer. The oven temperature was set to 55° C. and compound 6 dried to a constant weight for 12 hrs. The product 5c was discharged (12.70 Kg) in 76.5% yield (expected 85-95%). HPLC shows 98.96% purity and ¹H NMR confirmed the structure for compound 5c. ¹H NMR (DMSO): δ 11.10 (s, 1H), 7.36 (d, 1H), 6.78 (d, 2H), 6.75 (m, 1H), 6.56 (d, 2H), 6.20 (d, 1H), 5.18 (d, 2H), 2.76 (d, 3H).

### Step 4. 5-Chloro-N-(4-(6-fluoro-7-(methylamino)-2,4-dioxo-1,2-dihydroquinazolin-3 (4H)-yl)phenylcarbamoyl)thiophene-2-sulfonamide

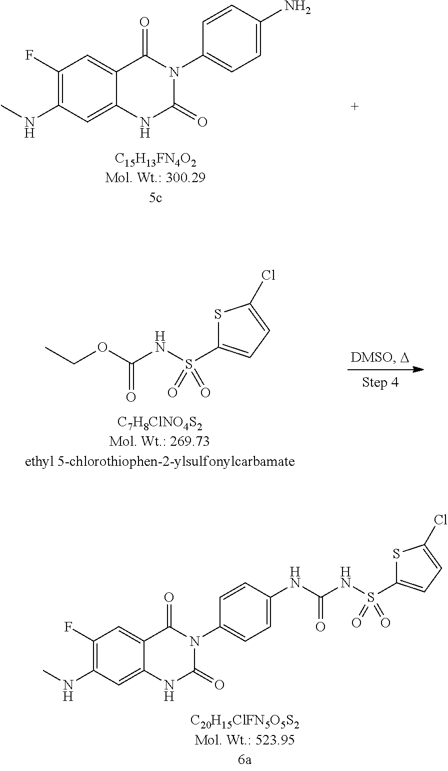

The PP1-R2000 (200 L HP reactor) reactor was charged with 6 (20.7 Kg, 1.0 equiv), Ethyl 5-chlorothiophene-2-ylsulfonylcarbamate (37.5 Kg, 2.0 equiv, >95%), dimethyl sulfoxide (>99%, 75 Kg) and agitated for 15 mins. While pulling maximum achievable vacuum, heated the 200 L HP reactor Number PP1-82000 at 65±5° C. for 15 hrs. Took the representative sample from the reactor for HPLC analysis, in-process HPLC indicated <0.9% compound 5c remaining in the reaction mixture (in-process criteria for reaction completion compound 6 <1%). Charged the 800 L reactor number PP5-R1000 with process filtered water (650 Kg) and then transferred the 200 L HP contents to the 800 L while maintaining the internal temperature below 25° C. The Rinsed the 200 L HP reactor with dimethyl sulfoxide (15 Kg) and transfer to the 800 L reactor which was then agitated for 2 hrs at 5±5° C. The solid formed was filtered through filter PP-F2000 to a 200 L GL receiver under vacuum and rinsed the filter cake with process filtered water (60 Kg). Took a representative sample of the wet cake and did HPLC analysis, if the purity of compound 6a is <95% (in-process control <95% the dichloromethane trituration d). The 800 L GL reactor was charged with all the wet compound 6a, dichloromethane (315 Kg) and agitated the contents for 3 hrs. The solid was filtered through GL nutsche filter lined with 1 sheet of T515 LF TYPAR filter under vacuum. The filter cake was washed with dichloromethane (50 Kg) and blow dried the cake with 8±7 psig of nitrogen for 15 mins. Transferred the filter cake into pre-lined vacuum trays with Dupont fluorocarbon film (Kind 100A) and then into the vacuum oven tray dryer set at 60° C. for 12 hrs. The dried compound 6a was isolated (33.6 Kg, 93% yield) with HPLC purity of 93.5% and 4.3% of sulfonamide. ¹H NMR confirmed the structure for compound 7. ¹H NMR (DMSO): δ 11.20 (s, 1H), 9.15 (s, 1H), 7.68 (d, 1H), 7.42 (d, 2H), 7.36 (d, 1H), 7.26 (m, 1H), 7.16 (d, 2H), 6.78 (m, 1H), 6.24 (d, 1H), 2.78 (d, 3H).

### Step 5. Potassium (5-chlorothiophen-2-ylsulfonyl)(4-(6-fluoro-7-(methylamino)-2,4-dioxo-1,2-dihydroquinazolin-3(4H)-yl)phenylcarbamoyl)amide, 7a

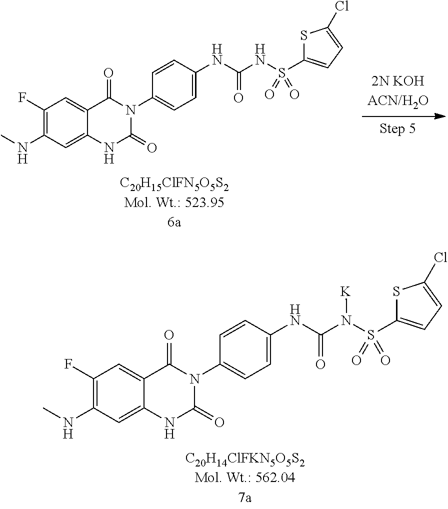

The 800 L GL reactor number PP5-R1000 was charged with acetonitrile (134 Kg), WFI quality water (156 Kg) and agitated the contents for 5 mins. To this then charged compound 6a (33.6 Kg, 1.0 equiv) and the reaction mixture was a suspension at this point. The suspension was charged with aqueous solution (WFI water, 35 Kg) of potassium hydroxide (4.14 Kg, 1.15 equiv, >85%) at a rate that maintains the internal temperature below 30° C. The charging lines were rinsed with WFI quality water (2 Kg) followed by heating the 800 L GL reactor contents to 50±5° C. for 1 hr. The contents were then filtered hot through a bag filter, then a seven cartridge 0.2μ polish filter to clean HDPE drums. The hot filtration system was maintained through out the filtration process so no material crashes out of the solution. Cool the 800 L GL reactor jacket to 25±5° C. before proceeding to the reactor rinse. Rinsed the 800 L GL reactor with pre-mixed solution of acetonitrile (8.5 Kg) and WFI quality water (10 Kg) through the filter system into the drums labeled as 7a hot filtration. Using the pressure vessel the 800 L GL reactor was rinsed with WFI quality water (20 Kg) followed by acetone (20 Kg) then blow it dry with nitrogen (3+2 psig). The 800GL reactor bottom valve was closed and pulled 20+10 inches Hg of vacuum, then break the vacuum and charge the reactor with the contents of the drums labeled as 7a hot filtration. Cooled the 800 L GL reactor number PP5-R1000 contents to 20±5° C. and then using a polish filter (PP-PF09), charged the reactor with methanol (373 kg, >99%) maintaining the internal temperature below 30° C. The contents of the 800GL reactor number PP5-R1000 were cooled to 15±5° C. followed by agitation of the contents for 12 hrs at this temperature. During this time the filterable solids were filtered through a clean filter apparatus (PP-F1000) into clean 200 L GL receiver (PPR-04) followed by pressurizing the reactor, pulled 20+10 inches Hg of vacuum on the filter/receiver and filtered the contents. The filter cake was washed with methanol (30 Kg) and blow dried with 8+7 psig of nitrogen for 10 mins. The vacuum oven tray dryer temperature was set to 80° C. prior to loading the wet cake of 7a. Transferred the wet filter cake into the pre-lined vacuum trays with Dupont's fluorocarbon film—Kind 100A and clamped down the special oven paper (Kavon MeI Tuf paper) over the vacuum trays containing the product wet 7a and transferred to the vacuum oven tray dryer. Set the oven temperature to 80° C. and dry the wet 7a to a constant weight (constant weight is defined as tray reading at least 1 hr apart having the same weight within +50 g. The representative sample was analyzed for residual solvents (residual solvent specifications for API) and it met the specifications. The final API was subjected to equilibration with water (5-6%) for 12 hrs with a tray of WFI quality water present, then thoroughly turned and allowed to stand for an additional 12 hrs and finally subjected to KF analysis (5.5% water content). Transferred the 7-potassium (21.80 Kg, 60.6% yield) to double heavy-duty poly bags and stored in secondary containment. HPLC taken showed purity of 99.7% for 7a and ¹H NMR confirmed the structure for 7a. ¹H NMR (DMSO): δ 11.14 (s, 1H), 8.60 (s, 1H), 7.48 (m, 2H), 7.35 (d, 1H), 7.22 (d, 1H), 6.95 (m, 3H), 6.75 (m, 1H), 6.22 (d, 1H), 2.78 (d, 3H).

### Example 4

### Inhibition of ADP-Mediated Platelet Aggregation In Vitro

The effect of testing the compound for use according to the invention on ADP-induced human platelet aggregation was assessed in a 96-well microtiter assay (see generally the procedures in Jantzen, H. M. et al. (1999) Thromb. Hemost. 81:111-117) or standard cuvette light transmittance aggregometry using either human platelet-rich plasma (PRP) or human washed platelets.

For preparation of human platelet-rich plasma for aggregation assays, human venous blood was collected from healthy, drug-free volunteers into 0.38% sodium citrate (0.013 M, pH 7.0 final). Platelet-rich plasma (PRP) is prepared by centrifugation of whole blood at 160×g for 20 minutes at room temperature. The PRP layer is removed, transferred to a new tube, and the platelet count is adjusted, if necessary, to achieve a platelet concentration of ˜3×10⁸ platelets/ml using platelet-poor plasma (PPP). PPP is prepared by centrifugation of the remaining blood sample (after removal of PRP) for 20 minutes at 800×g. This preparation of PRP can subsequently be used for aggregation assays in either a 96-well plate or standard cuvette aggregometry.

For preparation of washed platelets, human venous blood is collected from healthy, drug-free volunteers into ACD (85 mM sodium citrate, 111 mM glucose, 71.4 mM citric acid) containing PGI₂ (1.25 ml ACD containing 0.2 μM PGI2 final; PGI₂ was from Sigma, St. Louis, Mo.). Platelet-rich plasma (PRP) is prepared by centrifugation at 160×g for 20 minutes at room temperature. Washed platelets are prepared by centrifuging PRP for 10 minutes at 730 g and resuspending the platelet pellet in CGS (13 mM sodium citrate, 30 mM glucose, 120 mM NaCl; 2 ml CGS/10 ml original blood volume) containing 1 U/ml apyrase (grade V, Sigma, St. Louis, Mo.). After incubation at 37° C. for 15 minutes, the platelets are collected by centrifugation at 730 g for 10 minutes and resuspended at a concentration of 3×10⁸ platelets/ml in Hepes-Tyrode's buffer (10 mM Hepes, 138 mM NaCl, 5.5 mM glucose, 2.9 mM KCl, 12 mM NaHCO₃, pH 7.4) containing 0.1% bovine serum albumin, 1 mM CaCl₂ and 1 mM MgCl₂. This platelet suspension is kept >45 minutes at 37° C. before use in aggregation assays.

For cuvette light transmittance aggregation assays, serial dilutions (1:3) of test compounds were prepared in 100% DMSO in a 96 well V-bottom plate (final DMSO concentration in the cuvette was 0.6%). The test compound (3 μl of serial dilutions in DMSO) was preincubated with PRP for 30-45 seconds prior to initiation of aggregation reactions, which were performed in a ChronoLog aggregometer by addition of agonist (5 or 10 μM ADP) to 490 μL of PRP at 37° C. In some cases, light transmittance aggregometry was performed using 490 4 μL of washed platelets (prepared as described above) at 37° C., and aggregation was initiated by addition of 5 μM ADP and 0.5 mg/ml human fibrinogen (American Diagnostics, Inc., Greenwich, Conn.). The aggregation reaction is recorded for ˜5 min, and maximum extent of aggregation is determined by the difference in extent of aggregation at baseline, compared to the maximum aggregation that occurs during the five minute period of the assay. Inhibition of aggregation was calculated as the maximum aggregation observed in the presence of inhibitor, compared to that in the absence of inhibitor. IC₅₀s were derived by non-linear regression analysis using the Prism software (GraphPad, San Diego, Calif.).

Inhibition of ADP-dependent aggregation was also determined in 96-well flat-bottom microtiter plates using a microtiter plate shaker and plate reader similar to the procedure described by Frantantoni et al., Am. J. Clin. Pathol. 94, 613 (1990). All steps are performed at room temperature. For 96-well plate aggregation using platelet-rich plasma (PRP), the total reaction volume of 0.2 ml/well includes 180 μl of PRP (˜3×108 platelets/ml, see above), 6 μl of either serial dilution of test compounds in 20% DMSO or buffer (for control wells), and 10 μl of 20×ADP agonist solution (100 μM). The OD of the samples is then determined at 450 nm using a microtiter plate reader (Softmax, Molecular Devices, Menlo Park, Calif.) resulting in the 0 minute reading. The plates are then agitated for 5 min on a microtiter plate shaker and the 5 minute reading is obtained in the plate reader. Aggregation is calculated from the decrease of OD at 450 nm at t=5 minutes compared to t=0 minutes and is expressed as % of the decrease in the ADP control samples after correcting for changes in the unaggregated control samples. IC₅₀s were derived by non-linear regression analysis.

For 96-well plate aggregation using washed platelets, the total reaction volume of 0.2 ml/well includes in Hepes-Tyrodes buffer/0.1% BSA: 4.5×10⁷ apyrase-washed platelets, 0.5 mg/ml human fibrinogen (American Diagnostica, Inc., Greenwich, Conn.), serial dilutions of test compounds (buffer for control wells) in 0.6% DMSO. After ˜5 minutes preincubation at room temperature, ADP is added to a final concentration of 2 μM which induces submaximal aggregation. Buffer is added instead of ADP to one set of control wells (ADP-control). The OD of the samples is then determined at 450 nm using a microtiter plate reader (Softmax, Molecular Devices, Menlo Park, Calif.) resulting in the 0 minute reading. The plates are then agitated for 5 min on a microtiter plate shaker and the 5 minute reading is obtained in the plate reader. Aggregation is calculated from the decrease of OD at 450 nm at t=5 minutes compared to t=0 minutes and is expressed as % of the decrease in the ADP control samples after correcting for changes in the unaggregated control samples. IC₅₀s were derived by non-linear regression analysis.

### II. Inhibition of [3H]2-MeS-ADP Binding to Platelets

1. The Ability of Candidate Molecules to Inhibit the Binding of [3H]2-MeS-ADP to the P2_{Y12} Receptor on Platelets was Determined using a Radioligand Binding Assay.

Utilizing this assay the potency of inhibition of such compounds with respect to [³H]2-MeS-ADP binding to whole platelets is determined. Under the conditions described in II (3) below, the binding of [³H]2-MeS-ADP is solely due to the interaction of this ligand with the P2_{Y12} receptor, in that all the specific binding measured in this assay is competable with a P2_{Y12} antagonist (i.e., the specific binding is reduced to background levels by competition with an excess of P2_{Y12} antagonist, with no competition of binding when a P2Y₁ antagonist is pre-incubated with the platelet preparation). [³H]2-MeS-ADP binding experiments are routinely performed with outdated human platelets collected by standard procedures at hospital blood banks. Apyrase-washed outdated platelets are prepared as follows (all steps at room temperature, if not indicated otherwise):

Outdated platelet suspensions are diluted with 1 volume of CGS and platelets pelleted by centrifugation at 1900×g for 45 minutes. Platelet pellets are resuspended at 3−6×10⁹ platelets/ml in CGS containing 1 U/ml apyrase (grade V, Sigma, St. Louis, Mo.) and incubated for 15 minutes at 37° C. After centrifugation at 730×g for 20 minutes, pellets are resuspended in Hepes-Tyrode's buffer containing 0.1% BSA (Sigma, St. Louis, Mo.) at a concentration of 6.66×10⁸ platelets/ml. Binding experiments are performed after >45 minutes resting of the platelets.

Alternatively, binding experiments are performed with fresh human platelets prepared as described in section I (Inhibition of ADP-Mediated Platelet Aggregation in vitro), except that platelets are resuspended in Hepes-Tyrode's buffer containing 0.1% BSA (Sigma, St. Louis, Mo.) at a concentration of 6.66×10⁸ platelets/mil. Very similar results are obtained with fresh and outdated platelets.

A platelet ADP receptor binding assay (ARB) using the tritiated potent agonist ligand [³H]2-MeS-ADP (Jantzen, H. M. et al. (1999) Thromb. Hemost. 81:111-117) has been adapted to the 96-well microtiter format. In an assay volume of 0.2 ml Hepes-Tyrode's buffer with 0.1% BSA and 0.6% DMSO, 1×10⁸ apyrase-washed platelets are preincubated in 96-well flat bottom microtiter plates for 5 minutes with serial dilutions of test compounds before addition of 1 nM [³H]2-MeS-ADP ([³H]2-methylthioadenosine-5′-diphosphate, ammonium salt; specific activity 20-50 Ci/mmole, obtained by custom synthesis from Amersham Life Science, Inc., Arlington Heights, Ill., or NEN Life Science Products, Boston, Mass.). Total binding is determined in the absence of test compounds. Samples for nonspecific binding may contain 10 □M unlabelled 2-MeS-ADP (RBI, Natick, Mass.). After incubation for 15 minutes at room temperature, unbound radioligand is separated by rapid filtration and two washes with cold (4-8° C.) Binding Wash Buffer (10 mM Hepes pH 7.4, 138 mM NaCl) using a 96-well cell harvester (Minidisc 96, Skatron Instruments, Sterling, Va.) and 8×12 GF/C glassfiber filtermats (Printed Filtermat A, for 1450 Microbeta, Wallac Inc., Gaithersburg, Md.). The platelet-bound radioactivity on the filtermats is determined in a scintillation counter (Microbeta 1450, Wallac Inc., Gaithersburg, Md.). Specific binding is determined by subtraction of non-specific binding from total binding, and specific binding in the presence of test compounds is expressed as % of specific binding in the absence of test compound dilutions. IC₅₀s were derived by non-linear regression analysis.

In the table below, activity in the PRP assay is provided as follows: +++, IC₅₀<10 μM; ++, 10 μM<IC₅₀<30 μM. Activity in the ARB assay is provided as follows: +++, IC₅₀<0.05 μM; ++, 0.05 μM<IC₅₀<0.5 μM.

TABLE 5

| Example No. | ARB Binding | PRP Activity |
|---|---|---|
| Example 2 | +++ | +++ |

### Example 5

### Synthesis of [4-(6-fluoro-7-methylamino-2,4-dioxo-1,4-dihydro-2H-quinazolin-3-yl)-phenyl]-5-chloro-thiophen-2-yl-sulfonylurea potassium salt (9a) (Amorphous Form)

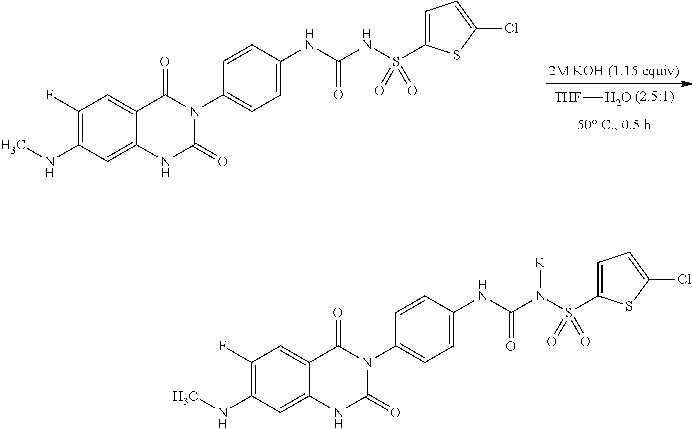

The free-acid, sulfonylurea, (7.0 g, 13.365 mmol) was suspended in THF/H₂O (55: 22 mL, ca. 2.5:1), and treated with 2M KOH (7.70 mL, 15.40 mmol, 1.15 equiv) drop wise, over ca. 5 min. By the time the addition was over, a clear solution resulted. But, then soon after (<5 mins), a solid precipitated out and reaction mixture became a heavy suspension.

This was heated in an oil-bath to 50° C., and the resulting clear viscous light brown solution was held there for 0.5 h. On cooling to it, the title compound precipitated out. The mixture was diluted with i-PrOH (250 mL, 3× the original reaction volume), stirred at rt. for 3 h, and then filtered through a Buchner funnel to yield the title compound as a colorless solid. This was dried in a vacuum oven at 80° C. to yield 7.20 g (96%) of an amorphous solid. MS (negative scan): 521.7; 523.7.

### Example 6

### Conversion of the sulfonylurea (7a) to its sodium salt (10a)

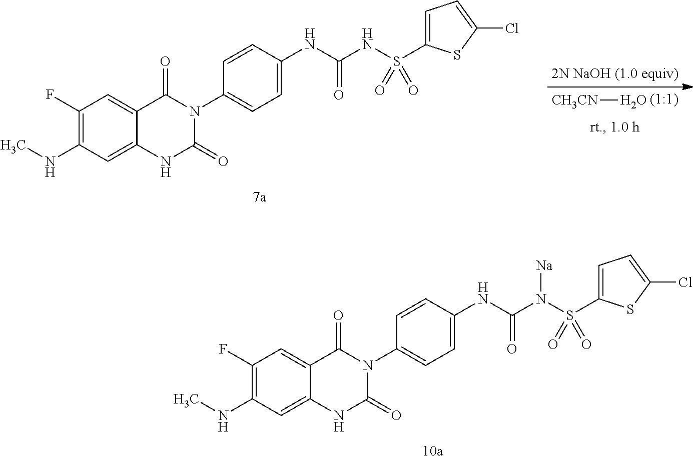

1-(5-chlorothiophen-2-ylsulfonyl)-3-(4-(6-fluoro-7-(methylamino)-2,4-dioxo-1,2-dihydroquinazolin-3(4H)-yl)phenyl)urea (3.0 g, 5.728 mmol) 7a was suspended in CH₃CN/H₂O) (1:1; 70 mL) and was treated with 2N NaOH (2.90 mL, 5.80 mmol), dropwise. Within ca. 15 minutes, a clear solution resulted. After stirring for 1.0 h, the now light brown solution was lyophilized to afford the crude product as an amorphous solid 10a. MS (negative scan): 522.0; 524.0.

### Example 7

### Preparation of Amorphous Form of the Sodium Salt

Sodium salt 10b was suspended in isopropanol (100 mL) and refluxed for ca. 45 min, then hot filtered to yield a tan solid, which is mostly the title compound by HPLC. The tan solid was suspended in CH₃CN:EtOH (1:2) (100 mL) and refluxed for 45 mins., then hot filtered to afford 2.54 g of the title compound as a tan solid (99.6887% pure by analytical HPLC, long column). The filtrate was diluted with EtOH until the ratio of ACN:EtOH became (1:3) and then let stand at room temperature overnight when the title compound precipitated out to afford 210 mg of the title compound (purity: 99.6685% by analytical HPLC, long column).

### Example 8

### Preparation of Polymorph Form a of Potassium Salt by Recrystallization

Recrystallization: The crude product can be recrystallized either from MeOH or MeOH/EtOH (3:1) by first heating to reflux to dissolve, and then cooling to room temperature to precipitate.

Recrystallization From MeOH: 1.0 g of the potassium salt was suspended in MeOH (150 mL) and heated to reflux for 0.5 h, resulting in an almost clear solution. This was then hot filtered through a Buchner funnel. The clear filtrate on standing at room temperature deposited a colorless solid. This was stirred overnight and then collected by filtration through a Buchner funnel. The solid product was rinsed with EtOH (2×4.0 mL) and dried in a vacuum oven at 80° C. for 20 h to yield 740 mg of a colorless solid. The mother liquor yielded more title compound on concentration to ca. one-third of the original volume.

Recrystallization from EtOH/MeOH: 1.0 g of the potassium salt was suspended in the solvent mixture EtOH/MeOH (1:3) (200 mL), and heated to reflux for 0.5 h resulting in an almost clear solution. This was then hot filtered through a Buchner funnel. The clear filtrate on standing at room temperature deposited a colorless solid. This was collected by filtration through a Buchner funnel. The solid product was rinsed with EtOH and dried in vacuum oven at 80° C. for 20 h to give a colorless solid. The mother liquor yielded more title compound upon concentration to ca. one-third of the original volume.

### Example 9

### Preparation of Polymorph Form B of Potassium Salt by Recrystallization

Recrystallization: The crude product can be recrystallized from EtOH/H₂O (91:9) or a small volume of MeOH by first heating to reflux to dissolve, and then cooling to room temperature to precipitate.

Recrystallization from EtOH/H₂O: 1.0 g of the potassium salt was suspended in EtOH (190 mL) and heated to reflux. To the heavy suspension was added H₂O (18.0 mL) dropwise, resulting in a clear colorless solution. On cooling to room temperature, the title compound precipitated out as a colorless solid. It was collected by filtration through a Buchner funnel, and rinsed with EtOH (2×4.0 mL). This was dried in vacuum oven at 80° C. for 20 h, to give 650 mg of a colorless solid. The mother liquor yielded more title compound upon concentration to ca. one-third of the original volume.

Large Scale Recrystallization from small volume of MeOH: 6.6 g of the potassium salt was suspended in MeOH (30 mL) and heated to reflux for 51u., the solid did not completely dissolve in less volume of methanol. After cooling the solid was filtered and rinsed with iPrOH. This was dried in vacuum oven at 80° C. for 20 h, to give 6.2 g of colorless solid, characterized to be Form B.

### Example 10

### Methods for Pharmacodynamic Assays

### Platelet Aggregation

Human venous blood was collected in a plastic syringe and immediately transferred to a plastic tube containing a fixed amount of anticoagulant (e.g., 5 μM (final) of a proprietary Portola anticoagulant C921-78 (a factor Xa inhibitor (see, Betz A, Wong P W, Sinha U. Inhibition of factor Xa by a peptidyl-alpha-ketothiazole involves 2 steps: evidence for a stabilizing conformational change. Biochemistry. 1999; 38: 14582-14591)) and mixed gently by inversion. Platelet-rich plasma (PRP) was prepared by centrifugation of whole blood at 160×g for 20 minutes at room temperature. The PRP layer was removed, transferred to a new tube, and the platelet count was adjusted, if necessary, to achieve a platelet concentration of ˜3×10⁸ platelets/ml using platelet-poor plasma (PPP). PPP is prepared by centrifugation of the remaining blood sample (after removal of PRP) for 20 minutes at 800×g. This preparation of PRP was used for standard light transmittance aggregometry assays. A fixed volume of PRP (0.3-0.5 mls) was transferred to an aggregometry cuvette, and the same volume of PPP was used to blank the machine. While stirring the PRP, a sufficient volume of adenosine diphosphate (ADP, Sigma-Aldrich) was added to achieve a final concentration of 10 μM in the cuvette, and the change in light transmittance was recorded for 6 minutes. The maximum extent of aggregation as well as the final (6 min after initiation of the aggregation reaction) extent of aggregation was determined from each reaction. Similarly for collagen aggregation assays, a final concentration of 4 μg/ml collagen (Chronolog corporation) was used to initiate the aggregation reaction; the change in light transmittance was recorded for 6 min, and the maximum extent of aggregation was determined from each reaction.

### Real Time Thrombosis Profiler (RTTP) Assay

Human venous blood was collected in a plastic syringe and immediately transferred to a plastic tube containing a fixed amount of anticoagulant (e.g., 5 μM (final) of a proprietary Portola anticoagulant C921-78) and mixed gently by inversion. Rhodamine 6G (1.25 μg/ml final concentration) was added to the blood, mixed by gentle inversion, and the tube was incubated at ˜37° C. for 20 min. Subsequently, the rhodamine-labeled blood was perfused through a rectangular glass capillary coated with type III collagen at an arterial shear rate (˜1600 sec⁻¹) and the extent of thrombus formation on the collagen surface was monitored for 5-7 min by the accumulation of fluorescently-labeled platelets using a video camera. The extent of the overall thrombotic process (e.g., accumulation of fluorescently-labeled platelets over time) was determined by analysis of various parameters derived from the curve generated from mean fluorescence intensity vs time (sec). These parameters may include slope, area under the curve, and endpoint.

### Example 11

### Tolerability and Pharmacokinetic and Pharmacodynamic Effects of Single Oral Liquid Doses of a Compound of Formula I in Human Subjects

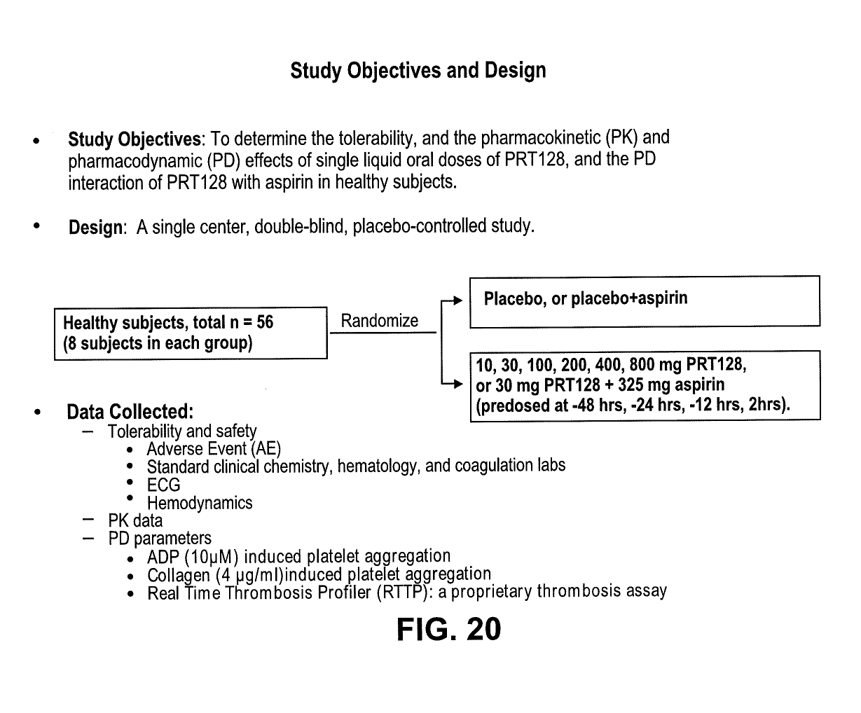

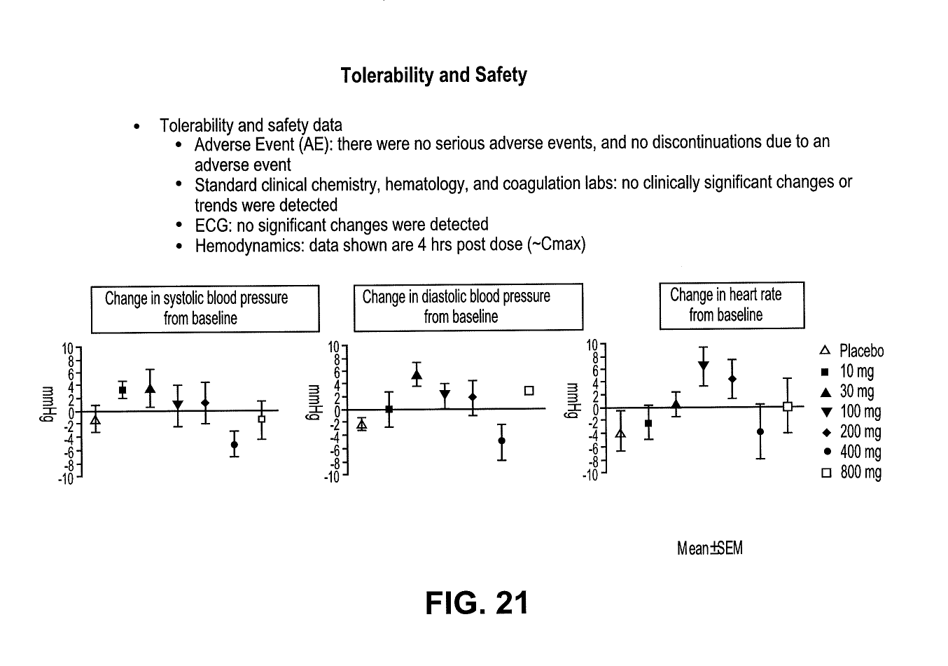

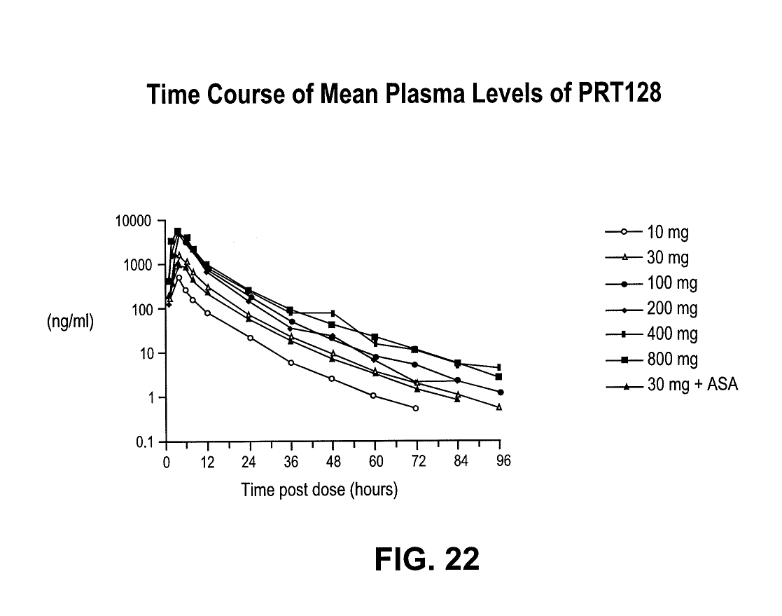

A single center, double-blind, placebo-controlled study of a compound of Formula I was conducted in human subjects. The study design is set forth in FIG. 20. The compound of Formula I was the potassium salt (Polymorph B) dissolved in water. The tolerability and safety results are presented in FIG. 21. The time course of mean plasma levels of the compound is shown in FIG. 22. The terminal half-life of the compound was about 12 hours. This half-life is consistent with a chronic oral dosage regimen of once, twice, or thrice a day to maintain plasma levels of the compound above its IC₅₀. The ability of the compound to inhibit ADP induced platelet aggregation is shown in FIG. 23. A dose of as little as 10 mg of the compound substantially inhibited ADP platelet aggregation as measured at six minutes in the ex vivo assay at a time point 4 hours after administration of the dose. Inhibition was dose dependent (see, FIG. 23B). Maximum (100%) inhibition of ADP-induced platelet aggregation as measured at six minutes was obtained at the higher dosages. FIG. 23C shows the effect on ADP-dependent platelet aggregation as measured at maximum amplitude. With regard to this less clinically relevant endpoint, the compound produced a substantial degree of inhibition even at the lowest dose of 10 mg. A maximum effect as measured at the four hour time point following oral administration with the drug was achieved at a dose of about 200 mg. FIG. 23D shows that the effect of the compound on platelet aggregation is reversible. The inhibitory effect (60%) observed at four hours post dose of a 100 mg dose which produced 60% inhibition at four hours was no longer observed at 24 hours post dose.

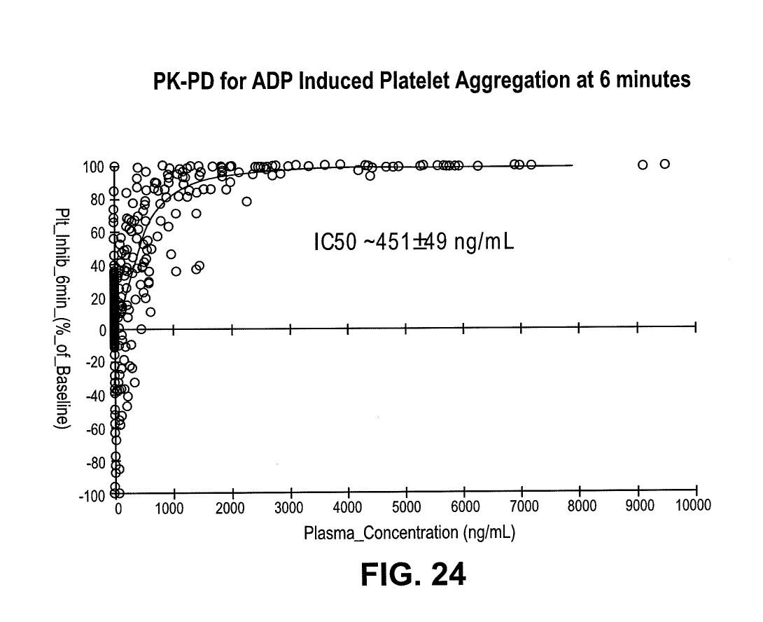

The relationship between plasma concentration of the compound and platelet inhibition is shown in FIG. 24. This figure indicates that the inhibition of ADP-induced platelet aggregation has an IC₅₀ of about 451 ng/ml and that concentrations of about 1000 to 2000 ng/ml give close to the maximum response.

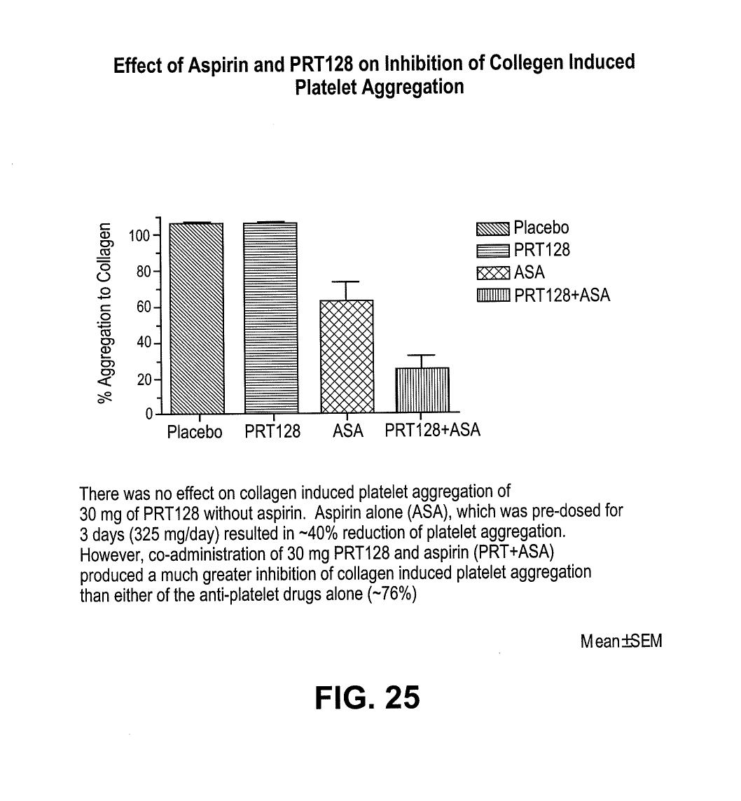

In a further aspect of the study, the effect of aspirin and the compound together on the inhibition of collagen-induced platelet aggregation was investigated. The compound of Formula I when given alone at a dose of 30 mg orally was without effect in the ex vivo collage induced platelet aggregation assay (see FIG. 25). The aspirin when predosed alone for three days at 325 mg/day resulted in about a 40% reduction in the assay. Administration of the compound together with aspirin produced a substantially greater inhibition of collagen-induced platelet aggregation, indicating there was substantial synergy in their interaction. Accordingly, in one aspect, the inventive methods provide for the treatment of a subject with both the compound for use according to the invention and aspirin.

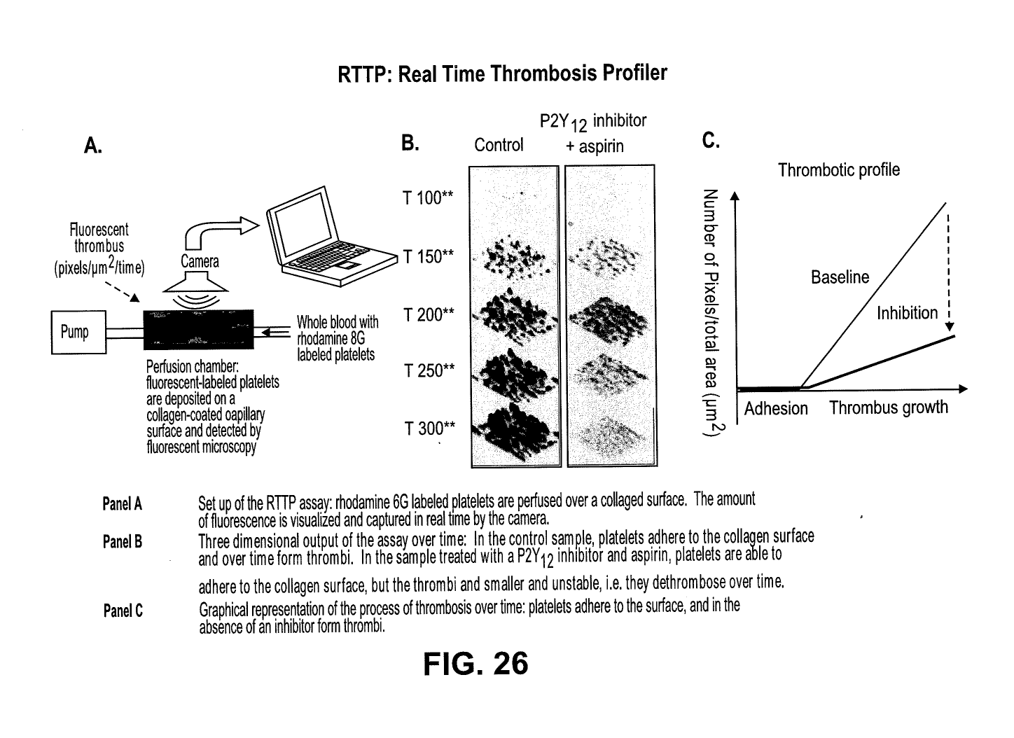

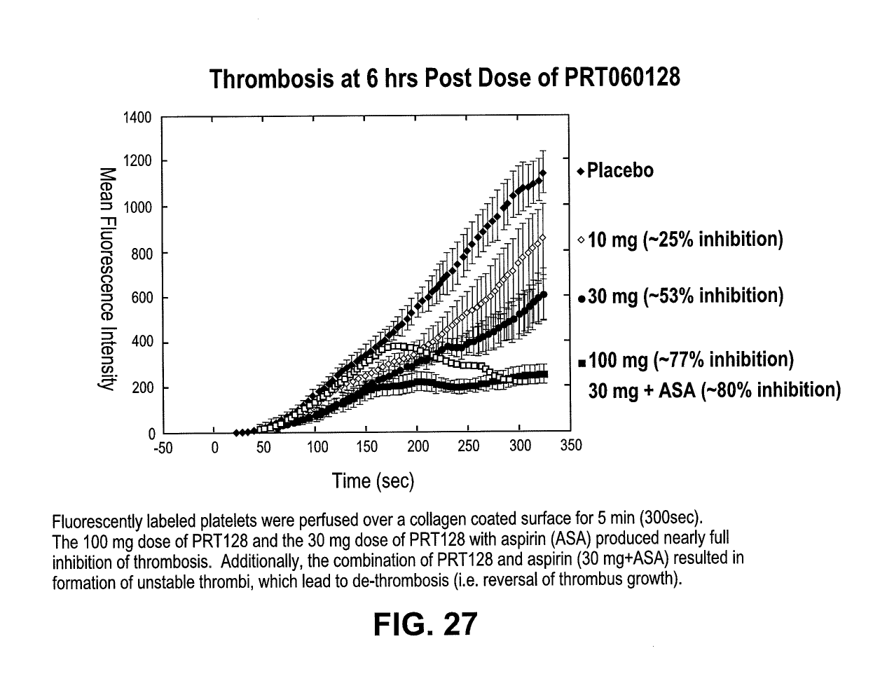

In yet another part of the study, the effects of the oral treatments in the human subjects was investigated using a Real Time Thrombosis Profiler (RTTP) as the ex vivo assay method. The set up and principles of operation of the RTTP are depicted in FIGS. 26A, B, and C. The results on thrombosis 6 hours post dosing with the compound of Formula I is shown in FIG. 27. There was a dose-dependent decrease in the RTTP collagen-induced platelet thrombosis for the subjects given the compound. A dose of 100 mg of the compound produced about a 70% inhibition. A dose of 30 mg of the compound produced a 53 percent inhibition. A dose of 30 mg of the compound in the subjects given aspirin produced nearly full inhibition of thrombosis and additionally resulted in the formation of unstable thrombi which lead to a shrinkage of the thrombi.

The results of Example 11 show that that the potassium salt of the compound for use according to the invention:
Can be well tolerated over a large range of oral doses (10-800 mg) in the human subjects. There were no serious adverse events, and no discontinuations due to an adverse event, hemodynamic, lab, or ECG change.
Can provide plasma PK increases which are dose proportional from 10-100 mg in the human subjects.
Can achieve dose dependent and full inhibition of ADP induced platelet aggregation in human subjects.
Can inhibit platelet aggregation following administration of 100 mg of the compound which is fully reversed by 24 hrs post dose in the human subjects.
Can provide good PK-PD correlation for platelet aggregation (IC₅₀≈450 ng/ml).
Appears to act synergistically with aspirin (325 mg) to inhibit collagen induced platelet aggregation and thrombosis. In RTTP, nearly complete inhibition of thrombosis was achieved following administration of either 100 mg of the compound, or 30 mg of the compound with aspirin.

### Example 12

### Tolerability and Pharmacokinetic and Pharmacodynamic Effects of Single Intravenous Doses of a Compound of Formula I in Human Subjects

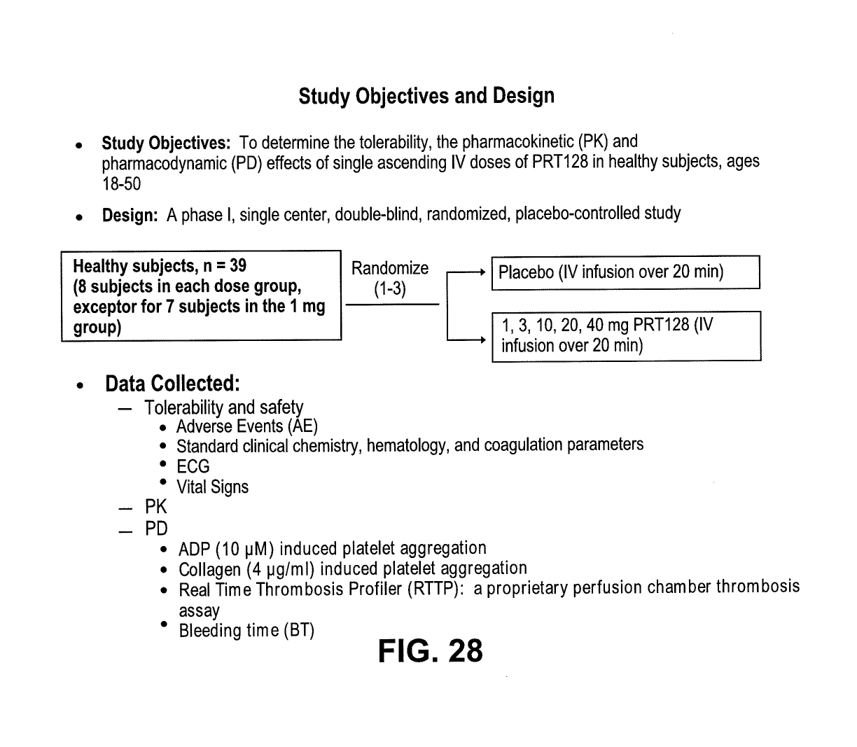

A single center, double-blind, placebo-controlled study of a compound of Formula I was conducted in human subjects. To determine the tolerability, the pharmacokinetic (PK) and pharmacodynamic (PD) effects of single ascending IV doses of a compound of Formula I (the potassium salt of polymorph B, or PRT128) in healthy subjects, ages 18-50. The study design is shown in FIG. 28.

Single IV doses of the compound, between 1 and 40 mg, were administered over 20 minutes to 5 groups of 8 healthy subjects (6 active, 2 placebo, except for 7 subjects in the 1 mg group) in a randomized, double-blind, study to determine tolerability, pharmacokinetic, and pharmacodynamic parameters. ADP (10 μM)-induced platelet aggregation was measured using 6-min endpoint and peak amplitude assays, as was platelet thrombosis on a collagen surface under a physiological shear rate using a proprietary perfusion chamber.

Results: With respect to off-target toxicity, all IV doses were well tolerated with no serious or clinically significant adverse events. No serious adverse events, and no discontinuations due to an adverse event were observed. Standard clinical chemistry, hematology, and coagulation labs: no clinically significant changes or trends were detected. No significant changes were detected in the ECG. No significant changes or trends in vital signs were detected.

With respect to bleeding time, pre-defined bleeding time stopping criteria (bleeding time of >20 min and 3 fold prolongation from baseline) were reached at the 40 mg dose.

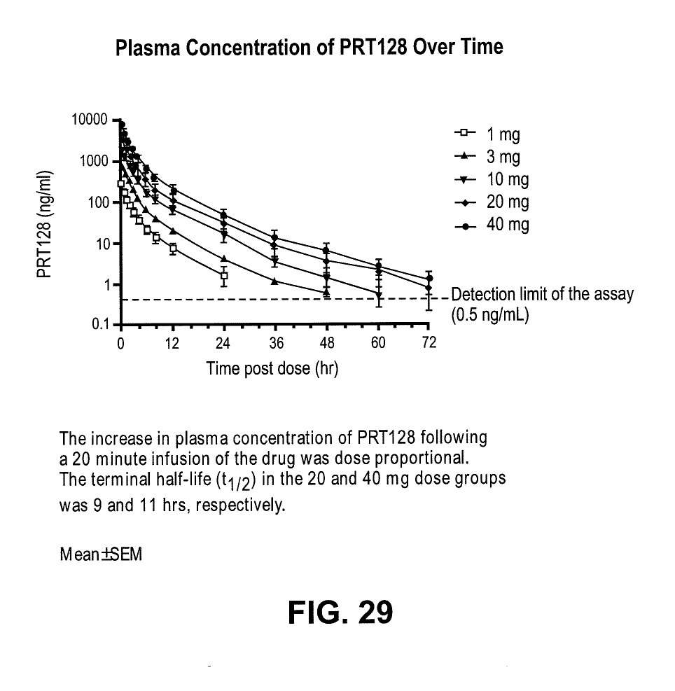

FIG. 29 shows that plasma drug concentration increases with dose.

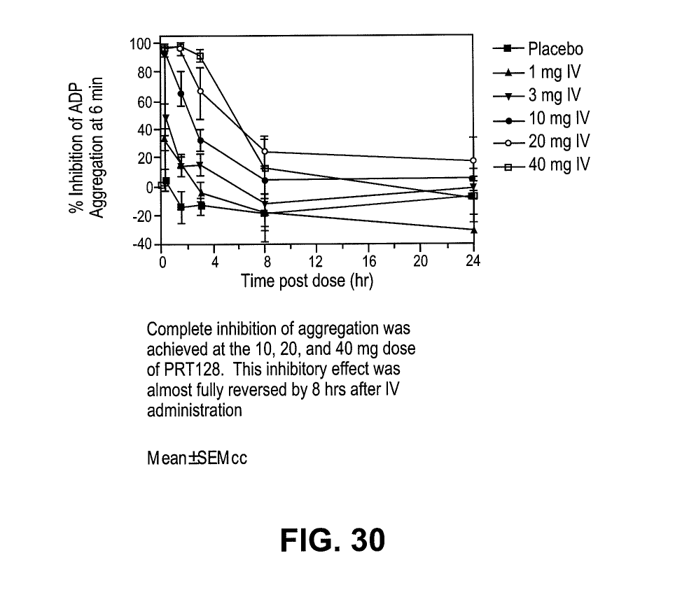

FIG. 30 shows the ability of the intravenously administered compound to inhibit ADP induced platelet aggregation. Maximum (100%) inhibition of ADP-induced platelet aggregation as measured at six minutes was obtained at the higher dosages. Complete inhibition of aggregation was achieved at the 10, 20, and 40 mg dose of PRT128. This inhibitory effect was almost fully reversed by 8 hrs after IV administration Inhibition was dose dependent.

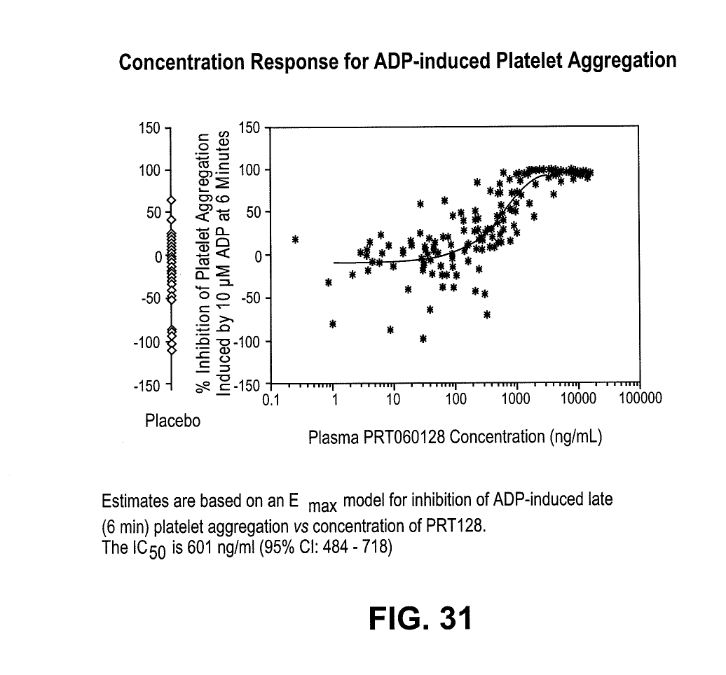

FIG. 31 depicts the concentration response for inhibition of ADP-induced platelet aggregation in plasma samples from the subjects. Estimates are based on an Eₘₐₓ model for inhibition of ADP-induced late (6 min) platelet aggregation vs the plasma concentration of PRT128. In this study, the IC₅₀ is 601 ng/ml (95% CI: 484-718 ng/ml).

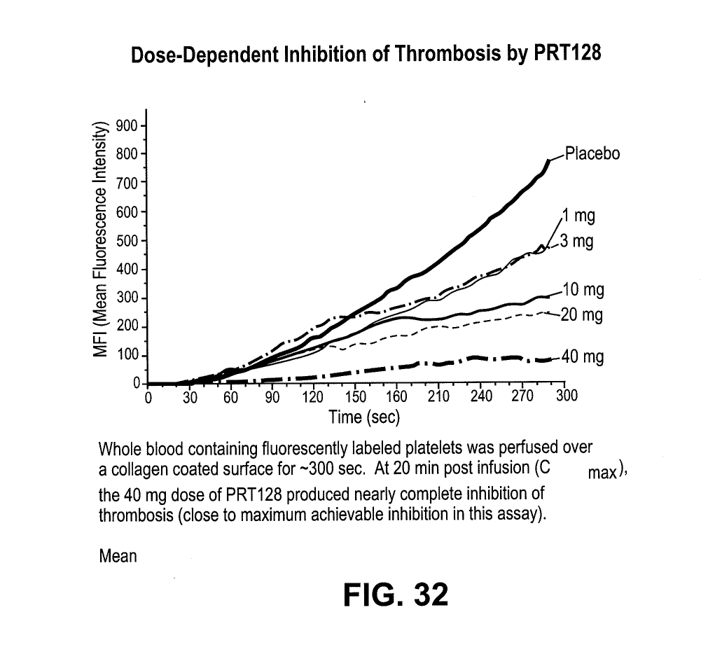

The dose-dependent inhibition of thromobosis was analyzed ex vivo using blood samples from the subjects (FIG. 32) in the Real Time Thrombosis Profiler. Whole blood containing fluorescently labeled platelets was perfused over a collagen coated surface for −300 sec. At 20 min post infusion (Cₘₐₓ), the 40 mg dose of PRT128 produced nearly complete inhibition of thrombosis (close to maximum achievable inhibition in this assay).

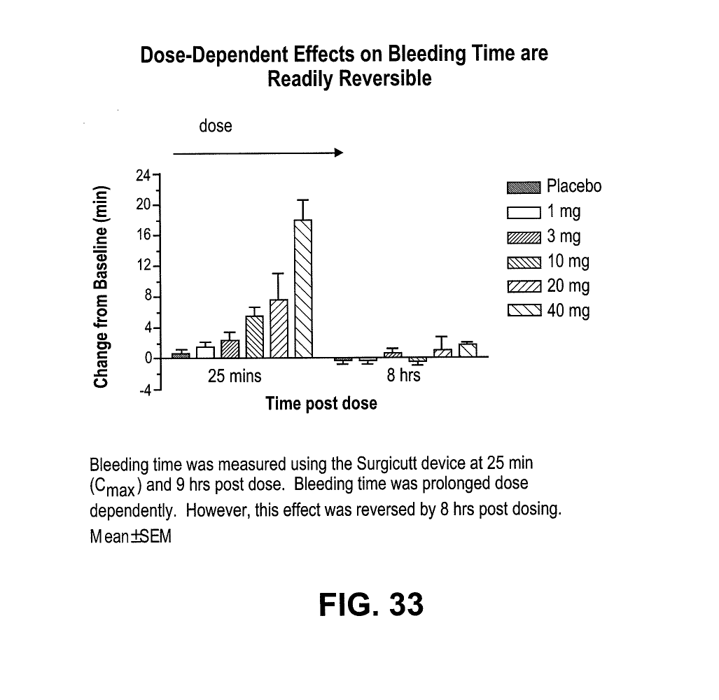

FIG. 33 shows that the dose-dependent effects on bleeding time are readily reversible over time. Bleeding time was measured using the Surgicutt device at 25 min (Cₘₐₓ) and 8 hrs post dose. Bleeding time was prolonged dose-dependently. However, this effect was reversed by 8 hrs post dosing.

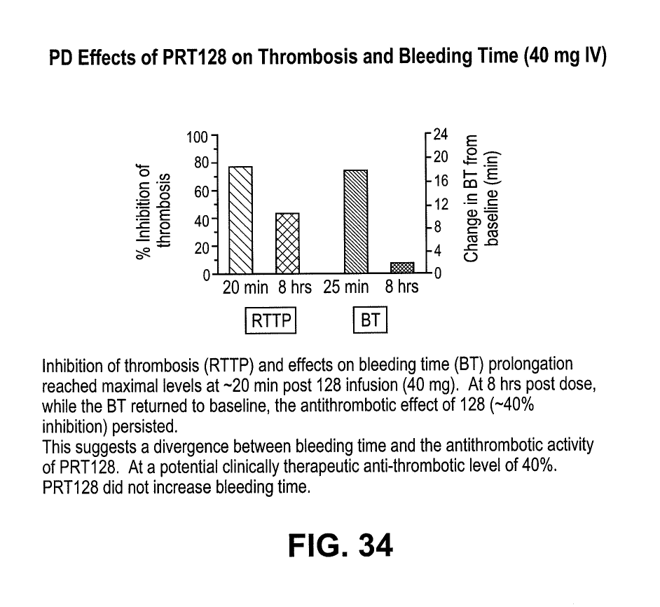

Inhibition of thrombosis (RTTP) and effects on bleeding time (BT) prolongation reached maximal levels at 20 min post 128 infusion (40 mg). At 8 hrs post dose, while the BT returned to baseline, the antithrombotic effect of the compound (−40% inhibition) persisted (see FIG. 34). This suggests a divergence between bleeding time and the antithrombotic activity of the compound. At a potential clinically therapeutic anti-thrombotic level of 40%, PRT128 did not increase bleeding time.

In summary, following IV administration:
The test compound achieved platelet inhibition that was essentially complete and immediate and so should be of benefit in treating subjects with ACS.
The inhibition was reversible and accordingly the compound will be of use in treating patients requiring a later and urgent surgical intervention.
The inhibition was reversible and so would be useful in treating patients requiring urgent surgical intervention.
All doses of the studied compound (1-40 mg) were well tolerated with no safety concerns or off-target toxicity. The studied compound has since been administered as IV bolus up to 45 mg and was equally well tolerated.
Drug exposure was dose proportional.
There was good PK-PD correlation for markers of platelet inhibition.
At a potential clinically therapeutic level, the studied compound achieved inhibition of thrombosis without substantially affecting bleeding time.

Conclusion: Administration of the studied compound achieved immediate, high-level platelet inhibition which correlated with plasma concentrations, and reached full inhibition of platelet aggregation and thrombosis at higher doses.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

What is claimed is:

1. A method of inhibiting ADP-induced platelet aggregation in a human subject in need thereof, said method comprising intravenously administering to the subject a pharmaceutical composition comprising a compound of the formula: 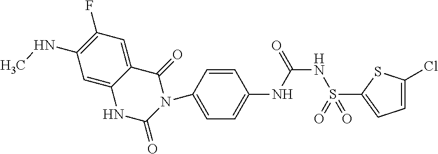
and at least one pharmaceutically acceptable excipient or carrier and wherein the composition is formulated for intravenous administration.

2. The method of claim 1, wherein the composition is formulated as a unit dose containing from 1 to 50 mg of the compound.

3. The method of claim 2, wherein the unit dose contains from 5 to 40 mg of the compound.

4. The method of claim 3, wherein the unit dose contains from 10 to 30 mg of the compound.

5. The method of claim 4, wherein the unit dose contains from 15 to 25 mg of the compound.

6. The method of claim 5, wherein the unit dose contains about 20 mg of the compound.

7. The method of claim 1, wherein the unit dose contains about 25 mg to 45 mg of the compound.

8. The method of claim 1, wherein the subject has an acute coronary syndrome.

9. The method of claim 1, wherein the subject is need of a reversible inhibition of ADP-induced platelet aggregation.

10. The method of claim 9, wherein the subject is to be scheduled for surgery or other medical procedure associated with bleeding within five days of the administration.

11. The method of claim 1, wherein the composition is administered as a bolus over a period of less than 20 minutes.

12. The method of claim 11, wherein the composition is administered as a bolus over a period of less than 10 minutes.

13. The method of claim 12, wherein the composition is administered as a bolus over a period of less than 5 minutes.

14. The method of claim 1, wherein the subject is further administered aspirin.

15. The method of claim 14, wherein the aspirin is administered orally.

16. The method of claim 1, wherein the subject was predosed with aspirin.

17. The method of claim 1, wherein the compound is formulated as a pharmaceutically acceptable salt.

18. The method of claim 17, wherein the salt is a sodium or potassium salt.

19. The method of claim 1, wherein a substantial degree of the platelet aggregation inhibition develops in the subject within 5 minutes after the composition is administered.

20. The method of claim 1, wherein a substantial degree of the platelet aggregation inhibition develops in the subject within 2 minutes after the composition is administered.

21. The method of claim 19, wherein the substantial degree of the platelet aggregation inhibition is at least 50% as determined by ADP-induced platelet aggregation values measured at six minutes.

22. The method of claim 19, wherein the substantial degree of the platelet aggregation inhibition is at least 70% as determined by ADP-induced platelet aggregation values measured at six minutes.

23. The method of claim 1, wherein the substantial degree of the platelet aggregation inhibition is at least 90% as determined by ADP-induced platelet aggregation values measured at six minutes.

24. The method of claim 1, wherein the inhibition is rapid in onset.

25. The method of claim 1, wherein a thrombolytic agent is also administered.

26. The method of claim 25, wherein the thrombolytic agent is TPA, SK, or TNK.

27. A method of inhibiting ADP-induced platelet aggregation inhibition in a human subject in need thereof, said method comprising orally administering to the subject a pharmaceutical composition comprising a compound of the formula: 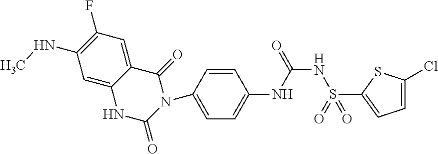
and at least one pharmaceutically acceptable excipient or carrier and wherein the composition is formulated for oral administration.

28. The method of claim 27, wherein the composition is formulated as a unit dose containing from 1 to 800 mg of the compound.

29. The method of claim 28, wherein the unit dose contains from 20 to 200 mg of the compound.

30. The method of claim 29, wherein the unit dose contains from 50 to 150 mg of the compound.

31. The method of claim 30, wherein the unit dose contains from 10 to 50 mg of the compound.

32. The method of claim 31, wherein the unit dose contains about 20 to 40 mg of the compound.

33. The method of claim 27, wherein the subject has an acute coronary syndrome.

34. The method of claim 27, wherein the subject is need of a reversible inhibition of ADP-induced platelet aggregation.

35. The method of claim 33, wherein the subject is to be scheduled for surgery or other medical procedure associated with bleeding within five days of the administration.

36. The method of claim 27, wherein the composition is formulated as a solid.

37. The method of claim 27, wherein the composition is administered as a tablet, capsule, or powder.

38. The method of claim 37, wherein the composition is administered as a liquid.

39. The method of claim 27, wherein the subject is further administered aspirin.

40. The method of claim 39, wherein the aspirin is administered orally.

41. The method of claim 27, wherein the subject was predosed with aspirin.

42. The method of claim 27, wherein the compound is formulated as a pharmaceutically acceptable salt.

43. The method of claim 42, wherein the salt is a sodium or potassium salt.

44. The method of claim 27, wherein a substantial degree of the platelet aggregation inhibition develops in the subject within 1 hour after the composition is administered.

45. The method of claim 27, wherein a substantial degree of the platelet aggregation inhibition develops in the subject within 2 hours after the composition is administered.

46. The method of claim 45, wherein the substantial degree of platelet aggregation inhibition is at least 50% as determined by ADP-induced platelet aggregation values measured at six minutes.

47. The method of claim 46, wherein the substantial degree of platelet aggregation inhibition is at least 70% as determined by ADP-induced platelet aggregation values measured at six minutes.

48. A pharmaceutical composition comprising a compound of the formula: 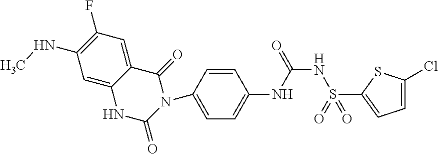
and at least one pharmaceutically acceptable excipient or carrier and wherein the composition is formulated for intravenous administration.

49. The composition of claim 48, wherein the composition is formulated as a unit dose containing from 1 to 50 mg of the compound.

50. The composition of claim 49, wherein the unit dose contains from 5 to 40 mg of the compound.

51. The composition of claim 50, wherein the unit dose contains from 10 to 30 mg of the compound.

52. The composition of claim 51, wherein the unit dose contains from 15 to 25 mg of the compound.

53. The composition of claim 52, wherein the unit dose contains about 20 mg of the compound.

54. A pharmaceutical composition comprising a compound of the formula: 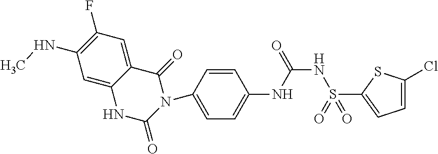
and at least one pharmaceutically acceptable excipient or carrier and wherein the composition is formulated for oral administration.

55. The composition of claim 54, wherein the composition is formulated as a unit dose containing from 1 to 800 mg of the compound.

56. The composition of claim 55, wherein the unit dose contains from 20 to 200 mg of the compound.

57. The composition of claim 56, wherein the unit dose contains from 50 to 150 mg of the compound.

58. The composition of claim 57, wherein the unit dose contains from 10 to 50 mg of the compound.

59. The composition of claim 58, wherein the unit dose contains about 20 to 40 mg of the compound.

60. A compound of the formula 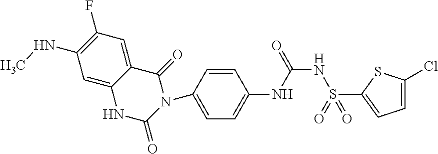
for use in manufacturing a medicament for treating ACS.
